(19) United States
(12) Patent Application Publication
(10) Pub. No.: US 2026/0132122 A1
(43) Pub. Date: May 14, 2026

(54) 6-6-FUSED BICYCLIC HETEROARYL AMINES AS CDK4 INHIBITORS

(71) Applicant: Incyte Corporation, Wilmington, DE (US)

(72) Inventors: Liana Hie, Wilmington, DE (US); Joshua Hummel, Hockessin, DE (US); Xiaolei Li, New Castle, DE (US); Sharada Manns, Wilmington, DE (US); Dibyendu Mondal, Wilmington, DE (US); Shaoqun Qian, Claymont, DE (US); Xiaozhao Wang, Moorestown, NJ (US); Bo Wei, Claymont, DE (US); Ming Xiang, Wilmington, DE (US)

(21) Appl. No.: 19/381,032

(22) Filed: Nov. 6, 2025

Related U.S. Application Data
(60) Provisional application No. 63/718,199, filed on Nov. 8, 2024.

(51) Int. Cl. C07D 401/14; A61K 31/517; A61K 31/519; A61K 31/5365; A61K 31/5377; A61P 35/00; C07D 405/14; C07D 417/14; C07D 471/04; C07D 498/04

(52) U.S. Cl. CPC C07D 401/14; A61K 31/517; A61K 31/519; A61K 31/5365; A61K 31/5377; A61P 35/00; C07D 405/14; C07D 417/14; C07D 471/04; C07D 498/04

(57) ABSTRACT

The present application provides compounds of Formula (I):

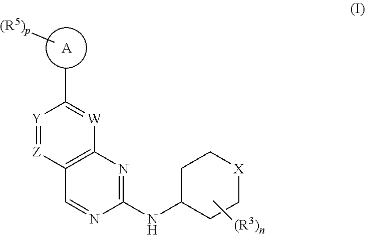

or a pharmaceutically acceptable salt thereof, that are inhibitors of cyclin-dependent kinase 4 (CDK4), as well as pharmaceutical compositions thereof, and methods of treating cancer using the same.

### CLAIM OF PRIORITY

This application claims the benefit of priority of U.S. Prov. Appl. No. 63/718,199, filed Nov. 8, 2024, which is incorporated herein by reference in its entirety.

### TECHNICAL FIELD

This application is directed to bicyclic amines which inhibit cyclin-dependent kinase 4 (CDK4) and are useful for treating cancer.

### BACKGROUND

Cyclin-dependent kinases (CDKs) are a family of serine/threonine kinases. Heterodimerized with regulatory subunits known as cyclins, CDKs become fully activated and regulate key cellular processes including cell cycle progression and cell division (Morgan, D. O. Annu. Rev. Cell Dev. Biol. 1997, 13:261-291). Uncontrolled proliferation is a hallmark of cancer cells. The deregulation of the CDK activity is associated with abnormal regulation of cell-cycle and is detected in virtually all forms of human cancers (Sherr, C. J. and Roberts, J. M., Genes Dev. 1999, 13(12): 1501-1512).

CDK4, in complex with cyclin D, is crucial for the transition from the G1 phase to the S phase of the cell cycle. The availability and synthesis of cyclin D are influenced by extracellular signals such as growth factors and mitogens (Sherr, C. J. and Roberts, J. M., Genes Dev. 1999, 13(12): 1501-1512; and Malumbres, M. et al. Nat. Rev. Cancer 2009, 9(3): 153-166). This transition is essential for DNA replication and cell division. One of the primary targets of the CDK4/cyclin D complex is the retinoblastoma protein (Rb). In its hypophosphorylated form, Rb binds to and inhibits E2F transcription factors (Weinberg, R. A. Cell 1995, 81(3): 323-330; and Dyson, N., Genes Dev. 1998, 12(15): 2245-2262). CDK4/cyclin D phosphorylates Rb, leading to its inactivation. Phosphorylation of retinoblastoma protein (Rb) by the CDK4/cyclin D complex leads to the release of E2F transcription factors, which promote the expression of genes required for DNA synthesis and S phase entry, such as those coding for cyclin E and other proteins involved in DNA replication (Harbour, J. W. and Dean, D. C., Genes Dev., 2000, 14(19): 2393-2409; Nevins, J. R., Hum. Mol. Genet., 2001, 10(7): 699-703). The action of CDK4/cyclin D ensure that once the cell passes the G1/S checkpoint, it is committed to completing the cell cycle, thus ensuring controlled and regulated cell division.

Overexpression or amplification of CDK4 can lead to abnormal activity, resulting in uncontrolled cell proliferation, a hallmark of cancer. The CDK4-cyclin D pathway is often deregulated in many cancers, leading to excessive cell division and tumor growth. Dysregulation of CCND1 in cancer can occur through gene amplification, chromosomal translocations, mutations in regulatory pathways, miRNA alterations, and changes in protein stability and degradation (Swerdlow, S. H. et al., Hum. Pathol., 1995 26(9): 999-1004; Knudsen, E. S. and Wang, J. Y, Mol. Cell Biol., 1997, 17(10): 5771-5783; Asghar, U., A. K. et al., Nat. Rev. Drug Discov., 2015, 14(2): 130-146; Zheng, N., Z. Wang and Wei, W., Int. J. Biochem. Cell Biol., 2016, 73: 99-110; and Lines, K. E. et al., J. Endocrinol., 2018, 240(1): 41-50). Additionally, mutations or alterations in the Rb pathway can enhance CDK4 activity, further contributing to cancer development (Weinberg, R. A. Cell 1995, 81(3): 323-330; Sherr, C. J. and Roberts, J. M., Genes Dev. 1999, 13(12): 1501-1512; Harbour, J. W. and Dean, D. C., Genes Dev., 2000, 14(19): 2393-2409; Nevins, J. R., Hum. Mol. Genet., 2001, 10(7): 699-703).

Due to its central role in cell cycle regulation, CDK4 is a target for cancer therapy. CDK4/6 dual inhibitors, such as palbociclib, ribociclib, and abemaciclib, have been developed and approved for the treatment of certain cancers, including breast cancer. These inhibitors work by blocking the kinase activity of CDK4/6, blocking cell cycle progression, and inducing senescence thereby inhibiting tumor growth (Asghar, U., A. K. et al., Nat. Rev. Drug Discov., 2015, 14(2): 130-146).

Further, a compound which is selective for CDK4 over CDK6 can be useful to minimize dose limiting toxicity. In particular, CDK4 is the primary oncogenic driver in certain breast cancers, the co-inhibition of CDK6-cyclin D3 can result in dose limiting toxicities due to associated hematological adverse events (Maurer, B. et al., Haematologica., 2021, 106(10): 2624-2632). Neutropenia is reported as the most common adverse events for CDK4/6 inhibitors, particularly palbociclib and ribociclib, and to a lesser extent abemaciclib (Liu, Y et al., BMC Cancer, 2023, 23(1): 816). Despite the clinical success of CDK4/6 inhibitors, such dose limiting toxicities limit the dosing regimen thus limiting effective target coverage. A selective CDK4 inhibitor would facilitate higher target coverage, thus maximizing efficacy while also minimizing the CDK6-associated toxicity profile, thus improving overall safety and tolerability. A next-generation CDK4 inhibitor could have clinical benefit in CDK4/6 refractory patients and could address acquired resistance to standard of care. Additionally, functional inhibition of CDK4, through a CDK4 molecule, has clinical opportunity in other indications besides breast cancers. CCND1 dysregulated cancers such as sarcomas, melanoma, head & neck, etc. could potentially benefit from a CDK4 inhibitor, either as a single agent or in combination with their respective standard of care.

These data provide a rationale for considering CDK4 as a potential target for new drug development in cancer associated with CDK4 and cyclin D expression and activity. In the last decade there has been increasing interest in the development of CDK selective inhibitors and there remains a need to discover CDK inhibitors having novel activity profiles, in particular those targeting CDK4 that spare off-target CDKs with associated toxicities, including CDK6 and CDK1 associated hematopoietic and/or gastrointestinal toxicities. This application is directed to this need and others.

### SUMMARY

The present invention relates to, inter alia, compounds of Formula (I):

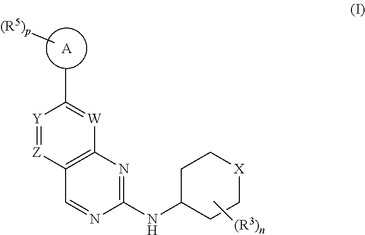

or pharmaceutically acceptable salts thereof, wherein the constituent members are defined herein.

The present invention further provides pharmaceutical compositions comprising a compound described herein, or a pharmaceutically acceptable salt thereof, and a pharmaceutically acceptable carrier.

The present invention further provides methods of inhibiting, comprising contacting the CDK4 with a compound described herein, or a pharmaceutically acceptable salt thereof.

The present invention further provides methods of inhibiting CDK4 in a patient, comprising administering to the patient a compound described herein, or a pharmaceutically acceptable salt thereof.

The present invention further provides methods of treating a disease or disorder associated with CDK4 in a patient, comprising administering to the patient a compound described herein, or a pharmaceutically acceptable salt thereof.

The present invention further provides compounds described herein, or a pharmaceutically acceptable salt thereof, for use in any of the methods described herein.

The present invention further provides uses of a compound described herein, or a pharmaceutically acceptable salt thereof, for the preparation of a medicament for use in any of the methods described herein.

### DETAILED DESCRIPTION

The present application provides, inter alia, a compound of Formula (I):

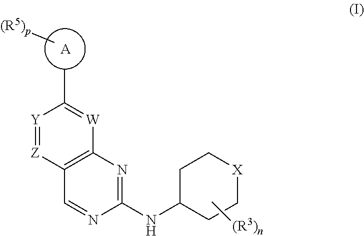

or a pharmaceutically acceptable salt thereof, wherein:
n is 0, 1, 2, 3, 4, 5, or 6;
p is 0, 1, 2, 3, or 4;
W is N or CR¹;
Y is N or CR²;
Z is N or CR⁶;
X is O, S, S(O)₂, S(O)NR^{c}, or N(-L-R⁴);
L is a bond, C₁₋₆ alkylene, C(O), C(O)NR^{a}, C(O)O, S(O)₂, S(O)(═NR^{b}), or S(O)₂NR^{a}; wherein C₁₋₆ alkylene is optionally substituted by 1, 2, 3 or 4 independently selected R^{G} substituents;
R^{a}, R^{b}, and R^{c} are each independently selected from H, C₁₋₆ alkyl, and C₁₋₆ haloalkyl;
or, alternatively, R^{a} and R⁴, taken together with the nitrogen atom to which they are attached, form a 4-7 membered heterocycloalkyl ring or 5-6 membered heteroaryl ring, each of which is optionally substituted by 1, 2, 3, or 4 independently selected R^{4A} substituents;
Ring A is 5-6 membered heteroaryl, having 1 to 2 heteroatom ring members;
R¹ is selected from H, D, halo, CN, C₁₋₆ alkyl, C₁₋₆ haloalkyl, C₃₋₁₀ cycloalkyl, 6-14 membered aryl, 4-15 membered heterocycloalkyl, 5-14 membered heteroaryl, C₃₋₁₀ cycloalkyl-C₁₋₄ alkyl, 6-14 membered aryl-C₁₋₄ alkyl, 4-15 membered heterocycloalkyl-C₁₋₄ alkyl, 5-14 membered heteroaryl-C₁₋₄ alkyl, OR^{b1}, SR^{b1}, NHOR^{a1}, C(O)R^{b1}, C(O)NR^{c1}R^{d1}, C(O)NR^{c1}(OR^{a1}), C(O)OR^{a1}, OC(O)R^{b1}, OC(O)NR^{c1}R^{d1}, NR^{c1}R^{d1}, NR^{c1}NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}C(═NR^{e1})R^{b1}, NR^{c1}S(O)NR^{c1}R^{d1}, NR^{c1}S(O)R^{b1}, NR^{c1}S(O)₂R^{b1}, NR^{c1}S(O)(═NR^{c1})R^{b1}, NR^{c1}S(O)₂NR^{c1}R^{d1}, S(O)R^{b1}, S(O)NR^{c1}R^{d1}, S(O)₂R^{b1}, S(O)₂NR^{c1}R^{d1}, OS(O)(═NR^{e1})R^{b1}, OS(O)₂R^{b1}, S(O)(═NR^{e1})R^{b1}, SF₅, P(O)R^{f1}R^{g1}, OP(O)(OR^{h1})(ORⁱ¹), P(O)(OR^{h1})(ORⁱ¹), and BR^{j1}R^{k1}, wherein said C₁₋₆ alkyl, C₁₋₆ haloalkyl, C₃₋₁₀ cycloalkyl, 6-14 membered aryl, 4-15 membered heterocycloalkyl, 5-14 membered heteroaryl, C₃₋₁₀ cycloalkyl-C₁₋₄ alkyl, 6-14 membered aryl-C₁₋₄ alkyl, 4-15 membered heterocycloalkyl-C₁₋₄ alkyl, and 5-14 membered heteroaryl-C₁₋₄ alkyl are each optionally substituted with 1, 2, 3, or 4 independently selected R^{1A} substituents;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1A} substituents;
or, any R^{c1} and R^{d1} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{1A} substituents;
each R^{b1} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{1A} substituents;
each R^{e1} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f1} and R^{g1} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h1} and Rⁱ¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j1} and R^{k1} is independently selected from OH, C₁₋₆ alkoxy, and C₁₋₆ haloalkoxy;
or any R^{j1} and R^{k1} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1A} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a1}, NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b11}, NR^{c11}S(O)NR^{c11}R^{d11}, NR^{c11}S(O)R^{b11}, NR^{c11}S(O)₂R^{b11}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, OS(O)₂R^{b11}, S(O)(═NR^{e11})R^{b11}, SF₅, P(O)R^{f11}R^{g11}, OP(O)(OR^{h11})(ORⁱ¹¹), P(O)(OR^{h11})(ORⁱ¹¹) and BR^{j11}R^{k11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{c11} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
or, any R^{c11} and R^{d11} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1B} substituents;
each R^{b11} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{c11} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f11} and R^{g11} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h11} and Rⁱ¹¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each Rⁱ¹¹ and R^{k11} is independently selected from OH, C₁₋₆ alkoxy, and C₁₋₆ haloalkoxy;
or any Rⁱ¹¹ and R^{k11} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1B} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, NHOR^{a12}, C(O)R^{b12}, C(O)NR^{c12}R^{d12}, C(O)NR^{c12}(OR^{a12}), C(O)OR^{a12}, OC(O)R^{b12}, OC(O)NR^{c12}R^{d12}, NR^{c12}R^{d12}, NR^{c12}NR^{c12}R^{d12}, NR^{c12}C(O)R^{b12}, NR^{c12}C(O)OR^{a12}, NR^{c12}C(O)NR^{c12}R^{d12}, C(═NR^{e12})R^{b12}, C(═NR^{e12})NR^{c12}R^{d12}, NR^{e12}C(═NR^{e12})NR^{c12}R^{d12}, NR^{c12}C(═NR^{e12})R^{b12}, NR^{c12}S(O)NR^{c12}R^{d12}, NR^{c12}S(O)R^{b12}, NR^{c12}S(O)₂R^{b12}, NR^{c12}S(O)(═NR^{e12})R^{b12}, NR^{c12}S(O)₂NR^{c12}R^{d12}, S(O)R^{b12}, S(O)NR^{c12}R^{d12}, S(O)₂R^{b12}, S(O)₂NR^{c12}R^{d12}, OS(O)(═NR^{e12})R^{b12}, OS(O)₂R^{b12}, S(O)(═NR^{e12})R^{b12}, SF₅, P(O)R^{f12}R^{g12}, OP(O)(OR^{h12})(ORⁱ¹²), P(O)(OR^{h12})(ORⁱ¹²), and BR^{j12}R^{k12}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a12}, R^{c12}, and R^{d12} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{e12} and R^{d12} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b12} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e12} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f12} and R^{g12} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h12} and Rⁱ¹² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j12} and R^{k12} is independently selected from OH, C₁₋₆ alkoxy, and C₁₋₆ haloalkoxy;
or any R^{j12} and R^{k12} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R² is selected from H, D, halo, CN, C₁₋₆ alkyl, C₁₋₆ haloalkyl, C₃₋₄ cycloalkyl, C₃₋₄ cycloalkyl-C₁₋₄ alkyl, OR^{b2}, SR^{b2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}C(═NR^{e2})R^{b2}, NR^{c2}S(O)NR^{c2}R^{d2}, NR^{c2}S(O)R^{b2}, NR^{c2}S(O)₂R^{b2}, NR^{e2}S(O)(═NR^{e2})R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}, wherein said C₁₋₆ alkyl, C₁₋₆ haloalkyl, C₃₋₄ cycloalkyl, and C₃₋₄ cycloalkyl-C₁₋₄ alkyl are each optionally substituted with 1, 2, 3, or 4 independently selected R^{G} substituents;
each R^{a2}, R^{c2}, and R^{d2} is independently selected from H, C₁₋₆ alkyl, C₁₋₆ haloalkyl, and C₃₋₄ cycloalkyl, wherein said C₁₋₆ alkyl, C₁₋₆ haloalkyl, and C₃₋₄ cycloalkyl are each optionally substituted with 1, 2, 3, or 4 independently selected R^{G} substituents;
each R^{b2} is independently selected from C₁₋₆ alkyl, C₁₋₆ haloalkyl, and C₃₋₄ cycloalkyl, which are each optionally substituted with 1, 2, 3, or 4 independently selected R^{G} substituents;
each R^{e2} is independently selected from H, OH, CN, C₁₋₆ alkyl, C₁₋₆ alkoxy, C₁₋₆ haloalkyl, and C₁₋₆ haloalkoxy;
each R³ is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}R^{d3}, NR^{c3}NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}C(═NR^{e3})R^{b3}, NR^{c3}S(O)NR^{c3}R^{d3}, NR^{c3}S(O)R^{b3}, NR^{c3}S(O)₂R^{b3}, NR^{c3}S(O)(═NR^{e3})R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a3}, R^{c3}, and R^{d3} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{b3} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e3} is independently selected from H, OH, CN, C₁₋₆ alkyl, C₁₋₆ alkoxy, C₁₋₆ haloalkyl, and C₁₋₆ haloalkoxy;
R⁴ is selected from H, C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl; wherein said C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted by 1, 2, 3, or 4 independently selected R^{4A} substituents;
each R^{4A} is independently selected from oxo, D, halo, CN, NO₂, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a41}, SR^{a41}, NHOR^{a41}, C(O)R^{b41}, C(O)NR^{c41}R^{d41}, C(O)NR^{c41}(OR^{a41}), C(O)OR^{a41}, OC(O)R^{b41}, OC(O)NR^{c41}R^{d41}, NR^{c41}R^{d41}, NR^{c41}NR^{c41}R^{d41}, NR^{c41}C(O)R^{b41}, NR^{c41}C(O)OR^{a41}, NR^{c41}C(O)NR^{c41}R^{d41}, C(═NR^{e41})R^{b41}, C(═NR^{e41})NR^{c41}R^{d41}, NR^{c41}C(═NR^{e41})NR^{c41}R^{d41}, NR^{c41}C(═NR^{e41})R^{b41}, NR^{c41}S(O)NR^{c41}R^{d41}, NR^{c41}S(O)R^{b41}, NR^{c41}S(O)₂R^{b41}, NR^{c41}S(O)(═NR^{e41})R^{b41}, NR^{c41}S(O)₂NR^{c41}R^{d41}, S(O)R^{b41}, S(O)NR^{c41}R^{d41}, S(O)₂R^{b41}, S(O)₂NR^{c41}R^{d41}, OS(O)(═NR^{e41})R^{b41}, and OS(O)₂R^{b41}, S(O)(═NR^{e41})R^{b41}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R⁴¹ substituents;
each R^{a41}, R^{c41}, and R^{d41} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl are each optionally substituted with 1, 2, 3, or 4 independently selected R^{4B} substituents;
or, any R^{c41} and R^{d41} attached to the same N atom, together with the N atom to which they are attached, form a 4-7 membered heterocycloalkyl group, which is optionally substituted with 1, 2, 3, or 4 independently selected R^{4B} substituents;
each R^{b41} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, which are each optionally substituted with 1, 2, 3, or 4 independently selected R^{4B} substituents;
each R^{e41} is independently selected from H, OH, CN, C₁₋₆ alkyl, C₁₋₆ alkoxy, C₁₋₆ haloalkyl, C₁₋₆ haloalkoxy, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl;
each R^{4B} is independently selected from D, halo, CN, NO₂, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a42}, SR^{a42}, NHOR^{a42}, C(O)R^{b42}, C(O)NR^{c42}R^{d42}, C(O)NR^{c42}(OR^{a42}), C(O)OR^{a42}, OC(O)R^{b42}, OC(O)NR^{c42}R^{d42}, NR^{c42}R^{d42}, NR^{c42}NR^{c42}R^{d42}, NR^{c42}C(O)R^{b42}, NR^{c42}C(O)OR^{a42}, NR^{c42}C(O)NR^{c42}R^{d42}, C(═NR^{e42})R^{b42}, C(═NR^{e42})NR^{c42}R^{d42}, NR^{e42}C(═NR^{e42})NR^{c42}R^{d42}, NR^{c42}C(═NR^{e42})R^{b42}, NR^{c42}S(O)NR^{c42}R^{d42}, NR^{c42}S(O)R^{b42}, NR^{c42}S(O)₂R^{b42}, NR^{c42}S(O)(═NR^{e42})R^{b42}, NR^{c42}S(O)₂NR^{c42}R^{d42}, S(O)R^{b42}, S(O)NR^{c42}R^{d42}, S(O)₂R^{b42}, S(O)₂NR^{c42}R^{d42}, OS(O)(═NR^{e42})R^{b42}, and OS(O)₂R^{b42}, S(O)(═NR^{e42})R^{b42}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a42}, R^{c42}, and R^{d42} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl are each optionally substituted with 1, 2, 3, or 4 independently selected R^{G} substituents;
each R^{b42} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, which are each optionally substituted with 1, 2, 3, or 4 independently selected R^{G} substituents;
each R^{e42} is independently selected from H, OH, CN, C₁₋₆ alkyl, C₁₋₆ alkoxy, C₁₋₆ haloalkyl, C₁₋₆ haloalkoxy, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl;
each R⁵ and R⁵¹ is independently selected from oxo, D, halo, NO₂, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a5}, SR^{a5}, NHOR^{a5}, C(O)R^{b5}, C(O)NR^{c5}R^{d5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}C(O)R^{b5}, NR^{c5}C(O)OR^{a5}, NRCC(O)NR^{c5}R^{d5}, C(═NR^{e5})R^{b5}, C(═NR^{e5})NR^{c5}R^{d5}, NR^{c5}C(═NR^{e5})NR^{c5}R^{d5}, NR^{c5}C(═NR^{e5})R^{b5}, NR^{c5}S(O)NR^{c5}R^{d5}, NR^{c5}S(O)R^{b5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{d5}, S(O)₂R^{b5}, S(O)₂NR^{c5}R^{d5}, OS(O)(═NR^{e5})R^{b5}, OS(O)₂R^{b5}, S(O)(═NR^{e5})R^{b5}, SF₅, P(O)R^{f5}R^{g5}, OP(O)(OR^{h5})(ORⁱ⁵), P(O)(OR^{h5})(ORⁱ⁵), and BR^{j5}R^{k5}; wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted by 1, 2, 3, or 4 independently selected R^{5A} substituents;
or, alternatively, any two R⁵ substituents on adjacent ring members, together with the atoms to which they are attached, form a 4-7 membered cycloalkyl or a 4-7 membered heterocycloalkyl ring, which are each optionally substituted by 1, 2, 3, or 4 independently selected R⁵¹ substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{5A} substituents;
or, any R^{c5} and R^{d5} attached to the same N atom, together with the N atom to which they are attached, form a 4-10 membered heterocycloalkyl group, which is optionally substituted with 1, 2, 3, or 4 independently selected R^{5A} substituents;
each R^{b5} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{5A} substituents;
each R^{e5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f5} and R^{g5} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h5} and Rⁱ⁵ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j5} and R^{k5} is independently selected from OH, C₁₋₆ alkoxy, and C₁₋₆ haloalkoxy;
or any R^{j5} and R^{k5} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{5A} is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a51}, SR^{a51}, NHOR^{a51}, C(O)R^{b51}, C(O)NR^{c51}R^{d51}, C(O)NR^{c51}(OR^{a51}), C(O)OR^{a51}, OC(O)R^{b51}, OC(O)NR^{c51}R^{d51}, NR^{c51}R^{d51}, NR^{c51}NR^{c51}R^{d51}, NR^{c51}C(O)R^{b51}, NR^{c51}C(O)OR^{a5}, NR^{c1}C(O)NR^{c51}R^{d51}, C(═NR^{e51})R^{b51}, C(═NR^{e51})NR^{c51}R^{d51}, NR^{c51}C(═NR^{e51})NR^{c51}R^{d51}, NR^{c51}C(═NR^{e51})R^{b51}, NR^{c51}S(O)NR^{c51}R^{d51}, NR^{c51}S(O)R^{b51}, NR^{c51}S(O)₂R^{b51}, NR^{c51}S(O)(═NR^{e51})R^{b51}, NR^{c51}S(O)₂NR^{c51}R^{d51}, S(O)R^{b51}, S(O)NR^{c51}R^{d51}, S(O)₂R^{b51}, S(O)₂NR^{c51}R^{d51}, OS(O)(═NR^{c51})R^{b51}, OS(O)₂R^{b51}, S(O)(═NR^{e51})R^{b51}, SF₅, P(O)R^{j51}R^{g51}, OP(O)(OR^{h51})(ORⁱ⁵¹), P(O)(OR^{h51})(ORⁱ⁵¹), and BR^{j51}R^{k51}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{G} substituents;
each R^{a51}, R^{c51}, and R^{d51} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{G} substituents;
or, any R^{c51} and R^{d51} attached to the same N atom, together with the N atom to which they are attached, form a 4-7 membered heterocycloalkyl group, which is optionally substituted with 1, 2, 3, or 4 independently selected R^{G} substituents;
each R^{b51} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{G} substituents;
each R^{e51} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f51} and R^{g51} are independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h51} and Rⁱ⁵¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j51} and R^{k51} is independently selected from OH, C₁₋₆ alkoxy, and C₁₋₆ haloalkoxy;
or any R^{j51} and R^{k51} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R⁶ is selected from H, D, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino; and
each R^{G} is independently selected from OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₄ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, n is 0, 1, or 2.

In some embodiments, n is 1 or 2.

In some embodiments, n is 0.

In some embodiments, n is 1.

In some embodiments, n is 2.

In some embodiments, p is 0, 1, or 2.

In some embodiments, p is 0 or 1.

In some embodiments, p is 0.

In some embodiments, p is 1.

In some embodiments, p is 2.

In some embodiments, W is N.

In some embodiments, W is CR¹.

In some embodiments, Y is N.

In some embodiments, Y is CR².

In some embodiments, Z is N.

In some embodiments, Z is CR⁶.

In some embodiments, X is O.

In some embodiments, X is S.

In some embodiments, X is S(O)₂.

In some embodiments, X is S(O)NR^{c}.

In some embodiments, X is N(-L-R⁴).

In some embodiments, X is O, S, S(O)₂, or N(-L-R⁴).

In some embodiments, X is O, S(O)₂, or N(-L-R⁴).

In some embodiments, X is O or N(-L-R⁴).

In some embodiments, L is a bond, C₁₋₄ alkylene, C(O), C(O)O, S(O)₂, or S(O)₂NR^{a}, wherein R^{a} is H, C₁₋₆ alkyl, or C₁₋₆ haloalkyl; or, alternatively, R^{a} and R⁴, taken together with the nitrogen atom to which they are attached, form a 4-7 membered heterocycloalkyl ring or 5-6 membered heteroaryl ring, each of which is optionally substituted by 1, 2, 3, or 4 independently selected R^{4A} substituents.

In some embodiments, L is a bond.

In some embodiments, L is C₁₋₆ alkylene.

In some embodiments, L is C₁₋₄ alkylene.

In some embodiments, L is CH₂.

In some embodiments, L is C(O).

In some embodiments, L is C(O)O.

In some embodiments, L is S(O)₂.

In some embodiments, L is S(O)₂NR^{a}, wherein R^{a} is H, C₁₋₆ alkyl, or C₁₋₆ haloalkyl, or, alternatively, R^{a} and R⁴, taken together with the nitrogen atom to which they are attached, form a 4-7 membered heterocycloalkyl ring or 5-6 membered heteroaryl ring, each of which is optionally substituted by 1, 2, 3, or 4 independently selected R^{4A} substituents.

In some embodiments, Ring A is 5-6 membered heteroaryl, having 1 to 2 heteroatom ring members, wherein at least 1 heteroatom is N.

In some embodiments, Ring moiety A is a 5-membered heteroaryl ring, having 1 to 2 heteroatom ring members, wherein at least 1 heteroatom is N.

In some embodiments, Ring moiety A is a 6-membered heteroaryl ring, having 1 to 2 heteroatom ring members, wherein at least 1 heteroatom is N.

In some embodiments, Ring moiety A is a pyrazole ring, an imidazole ring, a thiazole ring, an isothiazole ring, an oxazole ring, an isoxazole ring, a pyridine ring, or a pyrimidine ring.

In some embodiments, Ring moiety A is a pyrazole ring, an imidazole ring, a thiazole ring, or a pyridine ring.

In some embodiments, R¹ is selected from H, D, halo, CN, C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{b1}, SR^{b1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}, wherein said C₁₋₆ alkyl, C₁₋₆ haloalkyl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1A} substituents.

In some embodiments, R¹ is selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{b1}, SR^{b1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents.

In some embodiments, R¹ is selected from H, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, and OR^{b1}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents.

In some embodiments, R¹ is selected from H, halo, CN, C₁₋₆ alkyl, C₁₋₆ haloalkyl, C₃₋₇ cycloalkyl, phenyl, 4-7 membered heterocycloalkyl, 5-6 membered heteroaryl, and OR^{b1}, wherein said C₁₋₆ alkyl, C₁₋₆ haloalkyl, C₃₋₇ cycloalkyl, phenyl, 4-7 membered heterocycloalkyl, and 5-6 membered heteroaryl are each optionally substituted with 1 or 2 independently selected R^{1A} substituents.

In some embodiments:
each R^{a1}, R^{c1}, and R^{d1} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
each R^{b1} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1A} substituents;
each R^{1A} is independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄alkyl, OR^{a11}, SR^{a11}, C(O)R^{b11}, C(O)NR^{c11}R^{d11}, C(O)OR^{a11}, OC(O)R^{b11}OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{b11} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{1B} is independently selected from H, D, halo, CN, C₁₋₆ alkyl, C₂₋₆ alkenyl, C₂₋₆ alkynyl, C₁₋₆ haloalkyl, C₃₋₄ cycloalkyl, OR^{a12}, SR^{a12}, C(O)R^{b12}, C(O)NR^{c12}R^{d12}, C(O)OR^{a12}, OC(O)R^{b12}, OC(O)NR^{c12}R^{d12}, NR^{c12}R^{d12}, NR^{c12}C(O)R^{b12}, NR^{e12}C(O)OR^{a12}, NR^{e12}C(O)NR^{c12}R^{d12}, NR^{c12}S(O)₂R^{b12}, NR^{c12}S(O)₂NR^{c12}R^{d12}, S(O)₂R^{b12}, and S(O)₂NR^{c12}R^{d12}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{G} substituents;
each R^{a12}, R^{c12}, and R^{d12} is independently selected from H, C₁₋₆ alkyl, and C₁₋₆ haloalkyl; and
each R^{b12} is independently selected from C₁₋₆ alkyl and C₁₋₆ haloalkyl.

In some embodiments:
each R^{a1}, R^{c1}, and R^{d1} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
each R^{b1} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1A} substituents;
each R^{1A} is independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{1B} substituents;
each R^{b11} is independently selected from C₁₋₆ alkyl and C₁₋₆ haloalkyl, which are each optionally substituted with 1, 2, 3, or 4 independently selected R^{1B} substituents; and
each R^{1B} is independently selected from OH, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₄ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, R² is selected from H, D, halo, CN, C₁₋₆ alkyl, C₁₋₆ haloalkyl, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)₂R^{b2}, NR^{c2}S(O)₂NR^{c2}R^{d2}, S(O)₂R^{b2}, and S(O)₂NR^{c2}R^{d2}, wherein said C₁₋₆ alkyl and C₁₋₆ haloalkyl are each optionally substituted with 1, 2, 3, or 4 independently selected R^{G} substituents.

In some embodiments, R² is selected from H, D, halo, CN, C₁₋₆ alkyl, C₁₋₆ haloalkyl, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)₂R^{b2}, NR^{c2}S(O)₂NR^{c2}R^{d2}, S(O)₂R^{b2}, and S(O)₂NR^{c2}R^{d2}.

In some embodiments, R² is selected from H, D, halo, CN, C₁₋₆ alkyl, and C₁₋₆ haloalkyl.

In some embodiments, each R^{a2}, R^{c2}, and R^{d2} is independently selected from H, C₁₋₆ alkyl, C₁₋₆ haloalkyl, and C₃₋₄ cycloalkyl; and each R^{b2} is independently selected from C₁₋₆ alkyl, C₁₋₆ haloalkyl, and C₃₋₄ cycloalkyl.

In some embodiments, each R^{a2}, R^{c2}, and R^{d2} is independently selected from H, C₁₋₆ alkyl, and C₁₋₆ haloalkyl; and each R^{b2} is independently selected from C₁₋₆ alkyl and C₁₋₆ haloalkyl.

In some embodiments, each R³ is independently selected from D, halo, CN, C₁₋₆ alkyl, C₁₋₆ haloalkyl, C₃₋₄ cycloalkyl, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)₂R^{b3}, NR^{c3}S(O)₂NR^{c3}R^{d3}, S(O)₂R^{b3}, and S(O)₂NR^{c3}R^{d3}, wherein said C₁₋₆ alkyl, C₁₋₆ haloalkyl, and C₃₋₄ cycloalky are each optionally substituted with 1 or 2 independently selected R^{G} substituents.

In some embodiments, each R³ is independently selected from D, halo, CN, C₁₋₆ alkyl, C₁₋₆ haloalkyl, OR^{a3}, and NR^{c3}R^{d3}, wherein said C₁₋₆ alkyl and C₁₋₆ haloalkyl are each optionally substituted with 1 or 2 independently selected R^{G} substituents.

In some embodiments, each R³ is independently selected from —C₁₋₄ alkylene-OR^{a3}, —C₁₋₄ alkylene-NR^{c3}R^{d3}, OR^{a3}, and NR^{c3}R^{d3}.

In some embodiments, each R³ is independently selected from —C₁₋₄ alkylene-OH, —C₁₋₄ alkylene-NH₂, OH, and NH₂.

In some embodiments, each R³ is independently selected from —C₁₋₄ alkylene-OH and OH.

In some embodiments, each R^{a3}, R^{c3}, and R^{d3} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{G} substituents; and each R^{b3} is independently selected from C₁₋₆ alkyl and C₁₋₆ haloalkyl, which are each optionally substituted with 1, 2, 3, or 4 independently selected R^{G} substituents.

In some embodiments, each R^{a3}, R^{c3}, and R^{d3} is independently selected from H, C₁₋₆ alkyl, and C₁₋₆ haloalkyl; and each R^{b3} is independently selected from C₁₋₆ alkyl and C₁₋₆ haloalkyl.

In some embodiments, each R^{a3}, R^{c3}, and R^{d3} is H.

In some embodiments, R⁴ is selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl; wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{4A} substituents.

In some embodiments, R⁴ is selected from H, C₁₋₆ alkyl, C₁₋₆ haloalkyl, C₃₋₇ cycloalkyl, phenyl, 4-7 membered heterocycloalkyl, and 5-6 membered heteroaryl; wherein said C₁₋₆ alkyl, C₁₋₆ haloalkyl, C₃₋₇ cycloalkyl, phenyl, 4-7 membered heterocycloalkyl, and 5-6 membered heteroaryl are each optionally substituted by 1 or 2 independently selected R^{4A} substituents.

In some embodiments:
each R^{4A} is independently selected from oxo, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a41}, SR^{a41}, C(O)R^{b41}, C(O)NR^{c41}R^{d41}, C(O)OR^{a41}, OC(O)R^{b41}, OC(O)NR^{c41}R^{d41}, NR^{c41}R^{d41}, NR^{c41}C(O)R^{b41}, NR^{c41}C(O)OR^{a41}, NR^{c41}C(O)NR^{c41}R^{d41}, NR^{c41}S(O)₂R^{b41}, NR^{c41}S(O)₂NR^{c41}R^{d41}, S(O)₂R^{b41}, and S(O)₂NR^{c41}R^{d41}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{4B} substituents;
each R^{a41}, R^{c41}, and R^{d41} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{4B} substituents;
each R^{b41} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{4B} substituents;
each R^{4B} is independently selected from halo, CN, C₁₋₆ alkyl, C₁₋₆ haloalkyl, OR^{a42}, SR^{a42}, C(O)R^{b42}, C(O)NR^{c42}R^{d42}, C(O)OR^{a42}, OC(O)R^{b42}, OC(O)NR^{c42}R^{d42}, NR^{c42}R^{d42}, NR^{c42}C(O)R^{b42}, NR^{c42}C(O)OR^{a42}, NR^{c42}C(O)NR^{c42}R^{d42}, NR^{c42}S(O)₂R^{b42}, NR^{c42}S(O)₂NR^{c42}R^{d42}, S(O)₂R^{b42}, and S(O)₂NR^{c42}R^{d42};
each R^{a42}, R^{c42}, and R^{d42} is independently selected from H, C₁₋₆ alkyl, and C₁₋₆ haloalkyl; and
each R^{b42} is independently selected from C₁₋₆ alkyl and C₁₋₆ haloalkyl.

In some embodiments:
each R^{4A} is independently selected from oxo, halo, CN, C₁₋₆ alkyl, C₁₋₆ haloalkyl, C₃₋₇ cycloalkyl, phenyl, 4-7 membered heterocycloalkyl, 5-6 membered heteroaryl, OR^{a41}, SR^{a41}, C(O)R^{b41}, C(O)NR^{c41}R^{d41}, C(O)OR^{a41}, OC(O)R^{b41}, OC(O)NR^{c41}R^{d41}, NR^{c41}R^{d41}, NR^{c41}C(O)R^{b41}, NR^{c41}C(O)OR^{a41}, NR^{c41}C(O)NR^{c41}R^{d41}, NR^{c41}S(O)₂R^{b41}, NR^{c41}S(O)₂NR^{c41}R^{d41}, S(O)₂R^{b41}, and S(O)₂NR^{c41}R^{d41}, wherein said C₁₋₆ alkyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R^{4B} substituents;
each R^{a41}, R^{c41}, and R^{d41} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{4B} substituents;
each R^{b41} is independently selected from C₁₋₆ alkyl and C₁₋₆ haloalkyl, which are each optionally substituted with 1, 2, 3, or 4 independently selected R^{4B} substituents;
each R^{4B} is independently selected from halo, CN, C₁₋₆ alkyl, C₁₋₆ haloalkyl, OR^{a42}, C(O)R^{b42}, C(O)NR^{c42}R^{d42}, C(O)OR^{a42}, NR^{c42}R^{d42}, NR^{c42}C(O)R^{b42}, S(O)₂R^{b42}, and S(O)₂NR^{c42}R^{d42};
each R^{a42}, R^{c42}, and R^{d42} is independently selected from H, C₁₋₆ alkyl, and C₁₋₆ haloalkyl; and
each R^{b42} is independently selected from C₁₋₆ alkyl and C₁₋₆ haloalkyl.

In some embodiments:
each R⁵ and R⁵¹ is independently selected from oxo, D, halo, CN, C₁₋₆ alkyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5}, wherein said C₁₋₆ alkyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted by 1, 2, 3, or 4 independently selected R^{5A} substituents;
or, alternatively, any two R⁵ substituents on adjacent ring members, together with the atoms to which they are attached, form a 4-7 membered cycloalkyl or a 4-7 membered heterocycloalkyl ring, which are each optionally substituted by 1, 2, 3, or 4 independently selected R⁵¹ substituents.

In some embodiments:
each R⁵ and R⁵¹ is independently selected from oxo, D, halo, CN, C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5}; wherein said C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted by 1, 2, 3, or 4 independently selected R^{5A} substituents;
or, alternatively, any two R⁵ substituents on adjacent ring members, together with the atoms to which they are attached, form a 4-7 membered cycloalkyl or a 4-7 membered heterocycloalkyl ring, which are each optionally substituted by 1, 2, 3, or 4 independently selected R⁵¹ substituents.

In some embodiments:
each R⁵ and R⁵¹ is independently selected from oxo, D, halo, CN, C₁₋₆ alkyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5}, wherein said C₁₋₆ alkyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{5A} substituents;
or, alternatively, any two R⁵ substituents on adjacent ring members, together with the atoms to which they are attached, form a 4-7 membered cycloalkyl or a 4-7 membered heterocycloalkyl ring, which are each optionally substituted by 1, 2, 3, or 4 independently selected R⁵¹ substituents.

In some embodiments:
each R⁵ and R⁵¹ is independently selected from oxo, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a5}, SR^{a5}, C(O)R^{b5}, C(O)NR^{c5}R^{d5}, C(O)OR^{a5}, OC(O)R^{b5}OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5}; wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected RSA substituents;
or, alternatively, any two R⁵ substituents on adjacent ring members, together with the atoms to which they are attached, form a 4-7 membered cycloalkyl or a 4-7 membered heterocycloalkyl ring, which are each optionally substituted by 1, 2, 3, or 4 independently selected R⁵¹ substituents.

In some embodiments:
each R⁵ and R⁵¹ is independently selected from oxo, D, halo, CN, C₁₋₆ alkyl, C₂₋₃ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5}, wherein said C₁₋₆ alkyl, C₂₋₃ alkynyl, C₁₋₆ haloalkyl, C₃₋₇ cycloalkyl, phenyl, 4-7 membered heterocycloalkyl, and 5-6 membered heteroaryl are each optionally substituted by 1, 2, 3, or 4 independently selected R^{5A} substituents;
or, alternatively, any two R⁵ substituents on adjacent ring members, together with the atoms to which they are attached, form a 4-7 membered cycloalkyl or a 4-7 membered heterocycloalkyl ring, which are each optionally substituted by 1, 2, 3, or 4 independently selected R⁵¹ substituents.

In some embodiments:
each R⁵ and R⁵¹ is independently selected from oxo, D, halo, CN, C₁₋₆ alkyl, C₁₋₆ haloalkyl, C₃₋₇ cycloalkyl, phenyl, 4-7 membered heterocycloalkyl, 5-6 membered heteroaryl, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5}; wherein said C₁₋₆ alkyl, C₁₋₆ haloalkyl, C₃₋₇ cycloalkyl, phenyl, 4-7 membered heterocycloalkyl, and 5-6 membered heteroaryl are each optionally substituted by 1, 2, 3, or 4 independently selected R^{5A} substituents;
or, alternatively, any two R⁵ substituents on adjacent ring members, together with the atoms to which they are attached, form a 4-7 membered cycloalkyl or a 4-7 membered heterocycloalkyl ring, which are each optionally substituted by 1, 2, 3, or 4 independently selected R⁵¹ substituents.

In some embodiments:
each R⁵ and R⁵¹ is independently selected from oxo, D, halo, CN, C₁₋₆ alkyl, C₂₋₆ alkynyl, C₁₋₆ haloalkyl, C₃₋₆ cycloalkyl, phenyl, 4-7 membered heterocycloalkyl, C₃₋₇ cycloalkyl-C₁₋₄ alkyl, OR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5}, wherein said C₁₋₆ alkyl, C₂₋₆ alkynyl, C₁₋₆ haloalkyl, C₃₋₆ cycloalkyl, phenyl, and 4-7 membered heterocycloalkyl are each optionally substituted by 1 or 2 independently selected R^{5A} substituents;
or, alternatively, any two R⁵ substituents on adjacent ring members, together with the atoms to which they are attached, form a 4-7 membered cycloalkyl or a 4-7 membered heterocycloalkyl ring, which are each optionally substituted by 1, 2, 3, or 4 independently selected R⁵¹ substituents.

In some embodiments:
each R⁵ and R⁵¹ is independently selected from oxo, D, halo, CN, C₁₋₆ alkyl, C₁₋₆ haloalkyl, C₃₋₆ cycloalkyl, OR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5}; wherein said C₁₋₆ alkyl, C₁₋₆ haloalkyl, and C₃₋₆ cycloalkyl are each optionally substituted by 1 or 2 independently selected R^{5A} substituents;
or, alternatively, any two R⁵ substituents on adjacent ring members, together with the atoms to which they are attached, form a 4-7 membered cycloalkyl or a 4-7 membered heterocycloalkyl ring, which are each optionally substituted by 1, 2, 3, or 4 independently selected R⁵¹ substituents.

In some embodiments:
each R⁵ and R⁵¹ is independently selected from oxo, D, halo, CN, C₁₋₆ alkyl, C₂₋₆ alkynyl, C₁₋₆ haloalkyl, C₃₋₆ cycloalkyl, phenyl, 4-7 membered heterocycloalkyl, C₃₋₁₀ cycloalkyl-C₁₋₄ alkyl, OR^{a5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5}, wherein said C₁₋₆ alkyl, C₂₋₆ alkynyl, phenyl, and 4-7 membered heterocycloalkyl are each optionally substituted by 1 or 2 independently selected R^{5A} substituents;
or, alternatively, any two R⁵ substituents on adjacent ring members, together with the atoms to which they are attached, form a 4-7 membered cycloalkyl or a 4-7 membered heterocycloalkyl ring, which are each optionally substituted by 1, 2, 3, or 4 independently selected R⁵¹ substituents.

In some embodiments:
each R⁵ is independently selected from oxo, D, halo, CN, C₁₋₆ alkyl, C₁₋₆ haloalkyl, C₃₋₆ cycloalkyl, OR^{a5}, and NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5};
each R⁵¹ is independently selected from D, halo, CN, C₁₋₆ alkyl, C₁₋₆ haloalkyl, C₃₋₆ cycloalkyl, OR^{a5}, and NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5};
or, alternatively, any two R⁵ substituents on adjacent ring members, together with the atoms to which they are attached, form a 4-7 membered cycloalkyl or a 4-7 membered heterocycloalkyl ring, which are each optionally substituted by 1, 2, 3, or 4 independently selected R⁵¹ substituents.

In some embodiments:
each R^{a5}, R^{c5}, and R^{d5} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{5A} substituents;
each R^{b5} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{5A} substituents;
each R^{5A} is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a51}, SR^{a51}, C(O)R^{b51}, C(O)NR^{c51}R^{d51}, C(O)OR^{a51}, OC(O)R^{b51}, OC(O)NR^{c51}R^{d51}, NR^{c51}R^{d51}, NR^{c51}C(O)R^{b51} NR^{c51}C(O)OR^{a51}, NR^{c51}C(O)NR^{c51}R^{d51}, NR^{c51}S(O)₂R^{b51}, NR^{c51}S(O)₂NR^{c51}R^{d51}, S(O)₂R^{b51}, and S(O)₂NR^{c51}R^{d51}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a51}, R^{c51}, and R^{d51} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents; and
each R^{b51} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents.

In some embodiments:
each R^{a5}, R^{c5}, and R^{d5} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{5A} substituents;
each R^{b5} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{5A} substituents;
each R^{5A} is independently selected from D, halo, CN, C₁₋₆ alkyl, C₁₋₆ haloalkyl, C₃₋₅ cycloalkyl, 4-7 membered heterocycloalkyl-C₁₋₄ alkyl, OR^{a51}, SR^{a51}, C(O)R^{b51}, C(O)NR^{c51}R^{d51}, C(O)OR^{a51}, OC(O)R^{b51}, OC(O)NR^{c51}R^{d51}, NR^{c51}R^{d51}, NR^{c51}C(O)R^{b51}, NR^{c51}C(O)OR^{a51}, NR^{c51}C(O)NR^{c51}R^{d51}, NR^{c51}S(O)₂R^{b51}, NR^{c51}S(O)₂NR^{c51}R^{d51}, S(O)₂R^{b51}, and S(O)₂NR^{c51}R^{d51}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a51}, R^{c51}, and R^{d51} is independently selected from H, C₁₋₆ alkyl, and C₁₋₆ haloalkyl; and
each R^{b51} is independently selected from C₁₋₆ alkyl and C₁₋₆ haloalkyl.

In some embodiments:
each R^{a5}, R^{c5}, and R^{d5} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected RSA substituents;
each R^{b5} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{5A} substituents;
each R^{5A} is independently selected from D, halo, CN, C₁₋₆ alkyl, C₁₋₆ haloalkyl, C₃₋₅ cycloalkyl, OR^{a51}, SR^{a51}, C(O)R^{b51}, C(O)NR^{c51}R^{d51}, C(O)OR^{a51}, OC(O)R^{b51}, OC(O)NR^{c51}R^{d51}, NR^{c51}R^{d51}, NR^{c51}C(O)R^{b51}, NR^{c51}C(O)OR^{a51}, NR^{c51}C(O)NR^{c51}R^{d51}, NR^{c51}S(O)₂R^{b51}, NR^{c51}S(O)₂NR^{c51}R^{d51}, S(O)₂R^{b51}, and S(O)₂NR^{c51}R^{d51}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a51}, R^{c51}, and R^{d51} is independently selected from H, C₁₋₆ alkyl, and C₁₋₆ haloalkyl; and
each R^{b51} is independently selected from C₁₋₆ alkyl and C₁₋₆ haloalkyl.

In some embodiments:
each R^{a5}, R^{c5}, and R^{d5} is independently selected from H, C₁₋₆ alkyl, C₁₋₆ haloalkyl, C₃₋₅ cycloalkyl, and 4-6 membered heterocycloalkyl are each optionally substituted with 1, 2, 3, or 4 independently selected R^{5A} substituents;
each R^{b5} is independently selected from C₁₋₆ alkyl and C₁₋₆ haloalkyl, which are each optionally substituted with 1, 2, 3, or 4 independently selected R^{5A} substituents;
each R^{5A} is independently selected from D, halo, CN, C₁₋₆ alkyl, C₁₋₆ haloalkyl, 4-7 membered heterocycloalkyl-C₁₋₄ alkyl, OR^{a51}, and NR^{c51}R^{d51}; and
each R^{a51}, R^{c51}, and R^{d51} is independently selected from H, C₁₋₆ alkyl, and C₁₋₆ haloalkyl.

In some embodiments:
each R^{a5}, R^{c5}, and R^{d5} is independently selected from H, C₁₋₆ alkyl, C₁₋₆ haloalkyl, C₃₋₅ cycloalkyl, and 4-6 membered heterocycloalkyl are each optionally substituted with 1, 2, 3, or 4 independently selected R^{5A} substituents;
each R^{b5} is independently selected from C₁₋₆ alkyl and C₁₋₆ haloalkyl, which are each optionally substituted with 1, 2, 3, or 4 independently selected R^{5A} substituents;
each R^{5A} is independently selected from D, halo, CN, C₁₋₆ alkyl, C₁₋₆ haloalkyl, OR^{a51} and NR^{c51}R^{d51}; and
each R^{a51}, R^{c51}, and R^{d51} is independently selected from H, C₁₋₆ alkyl, and C₁₋₆ haloalkyl.

In some embodiments, R⁶ is selected from H, D, CN, halo, C₁₋₃ alkyl, and C₁₋₃ haloalkyl.

In some embodiments, each R^{G} is independently selected from OH, NO₂, CN, halo, C₁₋₃ alkyl, and C₁₋₃ haloalkyl.

In some embodiments:
n is 0, 1, or 2;
p is 0, 1, or 2;
W is N or CR¹;
Y is N or CR²;
Z is N or CR⁶;
X is O, S, S(O)₂, or N(-L-R⁴);
L is a bond, C₁₋₆ alkylene, C(O), C(O)O, S(O)₂, or S(O)₂NR^{a};
R^{a} is selected from H, C₁₋₆ alkyl, and C₁₋₆ haloalkyl;
or, alternatively, R^{a} and R⁴, taken together with the nitrogen atom to which they are attached, form a 4-7 membered heterocycloalkyl ring or 5-6 membered heteroaryl ring, each of which is optionally substituted by 1, 2, 3, or 4 independently selected R^{4A} substituents;
Ring A is 5-6 membered heteroaryl, having 1 to 2 heteroatom ring members, wherein at least 1 heteroatom is N;
R¹ is selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{b1}, SR^{b1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
each R^{a1}, R^{c1}, and R^{d1} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
each R^{b1} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1A} substituents;
each R^{1A} is independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄alkyl, OR^{a11}, SR^{a11}, C(O)R^{b11}, C(O)NR^{c11}R^{d11}, C(O)OR^{a11}, OC(O)R^{b11}OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{b11} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{1B} is independently selected from H, D, halo, CN, C₁₋₆ alkyl, C₂₋₆ alkenyl, C₂₋₆ alkynyl, C₁₋₆ haloalkyl, C₃₋₄ cycloalkyl, OR^{a12}, SR^{a12}, C(O)R^{b12}, C(O)NR^{c12}R^{d12}, C(O)OR^{a12}, OC(O)R^{b12}, OC(O)NR^{c12}R^{d12}, NR^{c12}R^{d12}, NR^{c12}C(O)R^{b12}, NR^{c12}C(O)OR^{a12}, NR^{c12}C(O)NR^{c12}R^{d12}, NR^{c12}S(O)₂R^{b12}, NR^{c12}S(O)₂NR^{c12}R^{d12}, S(O)₂R^{b12}, and S(O)₂NR^{c12}R^{d12}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{G} substituents;
each R^{a12}, R^{c12}, and R^{d12} is independently selected from H, C₁₋₆ alkyl, and C₁₋₆ haloalkyl;
each R^{b12} is independently selected from C₁₋₆ alkyl and C₁₋₆ haloalkyl;
R² is selected from H, D, halo, CN, C₁₋₆ alkyl, C₁₋₆ haloalkyl, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)₂R^{b2}, NR^{c2}S(O)₂NR^{c2}R^{d2}, S(O)₂R^{b2}, and S(O)₂NR^{c2}R^{d2};
R^{a2}, R^{c2}, and R^{d2} is independently selected from H, C₁₋₆ alkyl, C₁₋₆ haloalkyl, and C₃₋₄ cycloalkyl;
each R^{b2} is independently selected from C₁₋₆ alkyl, C₁₋₆ haloalkyl, and C₃₋₄ cycloalkyl;
each R³ is independently selected from D, halo, CN, C₁₋₆ alkyl, C₁₋₆ haloalkyl, C₃₋₄ cycloalkyl, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)₂R^{b3}, NR^{c3}S(O)₂NR^{c3}R^{d3}, S(O)₂R^{b3}, and S(O)₂NR^{c3}R^{d3}, wherein said C₁₋₆ alkyl, C₁₋₆ haloalkyl, and C₃₋₄ cycloalky are each optionally substituted with 1 or 2 independently selected R^{G} substituents.
each R^{a3}, R^{c3}, and R^{d3} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{G} substituents;
each R^{b3} is independently selected from C₁₋₆ alkyl and C₁₋₆ haloalkyl, which are each optionally substituted with 1, 2, 3, or 4 independently selected R^{G} substituents;
R⁴ is selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl; wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{4A} substituents;
each R^{4A} is independently selected from oxo, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a41}, SR^{a41}, C(O)R^{b41}, C(O)NR^{c41}R^{d41}, C(O)OR^{a41}, OC(O)R^{b41}, OC(O)NR^{c41}R^{d41}, NR^{c41}R^{d41}, NR^{c41}C(O)R^{b41}, NR^{c41}C(O)OR^{a41}, NR^{c41}C(O)NR^{c41}R^{d41}, NR^{c41}S(O)₂R^{b41}, NR^{c41}S(O)₂NR^{c41}R^{d41}, S(O)₂R^{b41}, and S(O)₂NR^{c41}R^{d41}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{4B} substituents;
each R^{a41}, R^{c41}, and R^{d41} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{4B} substituents;
each R^{b41} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{4B} substituents;
each R^{4B} is independently selected from halo, CN, C₁₋₆ alkyl, C₁₋₆ haloalkyl, OR^{a42}, SR^{a42}, C(O)R^{b42}, C(O)NR^{c42}R^{d42}, C(O)OR^{a42}, OC(O)R^{b42}, OC(O)NR^{c42}R^{d42}, NR^{c42}R^{d42}, NR^{c42}C(O)R^{b42}, NR^{c42}C(O)OR^{a42}, NR^{c42}C(O)NR^{c42}R^{d42}, NR^{c42}S(O)₂R^{b42}, NR^{c42}S(O)₂NR^{c42}R^{d42}, S(O)₂R^{b42}, and S(O)₂NR^{c42}R^{d42};
each R^{a42}, R^{c42}, and R^{d42} is independently selected from H, C₁₋₆ alkyl, and C₁₋₆ haloalkyl;
each R^{b42} is independently selected from C₁₋₆ alkyl and C₁₋₆ haloalkyl;
each R⁵ is independently selected from oxo, D, halo, CN, C₁₋₆ alkyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a5}, SR^{a5}, C(O)R^{b5}, C(O)NR^{c5}R^{d5}, C(O)OR^{a5}, OC(O)R^{b5}OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5}, wherein said C₁₋₆ alkyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{5A} substituents;
each R⁵¹ is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{5A} substituents;
or, alternatively, any two R⁵ substituents on adjacent ring members, together with the atoms to which they are attached, form a 4-7 membered cycloalkyl or a 4-7 membered heterocycloalkyl ring, which are each optionally substituted by 1, 2, 3, or 4 independently selected R⁵¹ substituents;
each R^{a5}, R^{c5}, and R^{d5} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{5A} substituents;
each R^{b5} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{5A} substituents;
each R^{5A} is independently selected from D, halo, CN, C₁₋₆ alkyl, C₁₋₆ haloalkyl, C₃₋₅ cycloalkyl, 4-7 membered heterocycloalkyl-C₁₋₄ alkyl, OR^{a51}, SR^{a51}, C(O)R^{b51}, C(O)NR^{c51}R^{d51}, C(O)OR^{a51}, OC(O)R^{b51}, OC(O)NR^{c51}R^{d51}, NR^{c51}R^{d51}, NR^{c51}C(O)R^{b51}, NR^{c51}C(O)OR^{a51}, NR^{c}C(O)NR^{c51}R^{d51}, NR^{c51}S(O)₂R^{b51}, NR^{c51}S(O)₂NR^{c51}R^{d51}, S(O)₂R^{b51}, and S(O)₂NR^{c51}R^{d51}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a51}, R^{c51}, and R^{d51} is independently selected from H, C₁₋₆ alkyl, and C₁₋₆ haloalkyl;
each R^{b51} is independently selected from C₁₋₆ alkyl and C₁₋₆ haloalkyl;
R⁶ is selected from H, D, CN, halo, C₁₋₃ alkyl, and C₁₋₃ haloalkyl; and
each R^{G} is independently selected from OH, CN, halo, C₁₋₃ alkyl, C₁₋₃ haloalkyl, cyano-C₁₋₃ alkyl, HO—C₁₋₃ alkyl, C₁₋₃ alkoxy-C₁₋₃ alkyl, C₃₋₄ cycloalkyl, C₁₋₃ alkoxy, C₁₋₃ haloalkoxy, amino, C₁₋₃ alkylamino, di(C₁₋₃ alkyl)amino, C₁₋₃ alkylsulfonyl, carbamyl, C₁₋₃ alkylcarbamyl, di(C₁₋₃ alkyl)carbamyl, C₁₋₃ alkylcarbonyl, C₁₋₃ alkoxycarbonyl, C₁₋₃ alkylcarbonyloxy, and C₁₋₃ alkylcarbonylamino.

In some embodiments:
n is 0, 1, or 2;
p is 0, 1, or 2;
W is N or CR¹;
Y is N or CR²;
Z is N or CR⁶;
X is O, S, S(O)₂, or N(-L-R⁴);
L is a bond, C₁₋₆ alkylene, C(O), C(O)O, S(O)₂, or S(O)₂NR^{a};
R^{a} is selected from H, C₁₋₆ alkyl, and C₁₋₆ haloalkyl;
or, alternatively, R^{a} and R⁴, taken together with the nitrogen atom to which they are attached, form a 4-7 membered heterocycloalkyl ring or 5-6 membered heteroaryl ring, each of which is optionally substituted by 1, 2, 3, or 4 independently selected R^{4A} substituents;
Ring A is 5-6 membered heteroaryl, having 1 to 2 heteroatom ring members, wherein at least 1 heteroatom is N;
R¹ is selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{b1}, SR^{b1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NRCC(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents.
each R^{a1}, R^{c1}, and R^{d1} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
each R^{b1} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1A} substituents;
each R^{1A} is independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)OR^{a11}, OC(O)R^{b11}OC(O)NR^{c11}R^{d11}, NR^{c11}R^{d11}, NR^{c11}C(O)R^{b11} NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{b11} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{1B} is independently selected from H, D, halo, CN, C₁₋₆ alkyl, C₂₋₆ alkenyl, C₂₋₆ alkynyl, C₁₋₆ haloalkyl, C₃₋₄ cycloalkyl, OR^{a12}, SR^{a12}, C(O)R^{b12}, C(O)NR^{c12}R^{d12}, C(O)OR^{a12}, OC(O)R^{b12}, OC(O)NR^{c12}R^{d12}, NR^{c12}R^{d12}, NR^{c12}C(O)R^{b12}, NR^{c12}C(O)OR^{a12}, NR^{c12}C(O)NR^{c12}R^{d12}, NR^{c12}S(O)₂R^{b12}, NR^{c12}S(O)₂NR^{c12}R^{d12}, S(O)₂R^{b12}, and S(O)₂NR^{c12}R^{d12}, wherein said C₁₋₆ alkyl, C₂₋₆ alkenyl, C₂₋₆ alkynyl, C₁₋₆ haloalkyl, and C₃₋₄ cycloalkyl are each optionally substituted with 1, 2, 3, or 4 independently selected R^{G} substituents;
each R^{a12}, R^{c12}, and R^{d12} is independently selected from H, C₁₋₆ alkyl, and C₁₋₆ haloalkyl;
each R^{b12} is independently selected from C₁₋₆ alkyl and C₁₋₆ haloalkyl;
R² is selected from H, D, halo, CN, C₁₋₆ alkyl, C₁₋₆ haloalkyl, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)₂R^{b2}, NR^{c2}S(O)₂NR^{c2}R^{d2}, S(O)₂R^{b2}, and S(O)₂NR^{c2}R^{d2};
R^{a2}, R^{c2}, and R^{d2} is independently selected from H, C₁₋₆ alkyl, C₁₋₆ haloalkyl, and C₃₋₄ cycloalkyl;
each R^{b2} is independently selected from C₁₋₆ alkyl, C₁₋₆ haloalkyl, and C₃₋₄ cycloalkyl;
each R³ is independently selected from D, halo, CN, C₁₋₆ alkyl, C₁₋₆ haloalkyl, C₃₋₄ cycloalkyl, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)₂R^{b3}, NR^{c3}S(O)₂NR^{c3}R^{d3}, S(O)₂R^{b3}, and S(O)₂NR^{c3}R^{d3}, wherein said C₁₋₆ alkyl, C₁₋₆ haloalkyl, and C₃₋₄ cycloalky are each optionally substituted with 1 or 2 independently selected R^{G} substituents.
each R^{a3}, R^{c3}, and R^{d3} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{G} substituents;
each R^{b3} is independently selected from C₁₋₆ alkyl and C₁₋₆ haloalkyl, which are each optionally substituted with 1, 2, 3, or 4 independently selected R^{G} substituents;
R⁴ is selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl; wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{4A} substituents;
each R^{4A} is independently selected from oxo, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a41}, SR^{a41}, C(O)R^{b41}, C(O)NR^{c41}R^{d41}, C(O)OR^{a41}, OC(O)R^{b41}, OC(O)NR^{c41}R^{d41}, NR^{c41}R^{d41}, NR^{c41}C(O)R^{b41}, NR^{c41}C(O)OR^{a41}, NR^{c41}C(O)NR^{c41}R^{d41}, NR^{c41}S(O)₂R^{b41}, NR^{c41}S(O)₂NR^{c41}R^{d41}, S(O)₂R^{b41}, and S(O)₂NR^{c41}R^{d41}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R⁴¹ substituents;
each R^{a41}, R^{c41}, and R^{d41} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{4B} substituents;
each R^{b41} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{4B} substituents;
each R^{4B} is independently selected from halo, CN, C₁₋₆ alkyl, C₁₋₆ haloalkyl, OR^{a42}, SR^{a42}, C(O)R^{b42}, C(O)NR^{c42}R^{d42}, C(O)OR^{a42}, OC(O)R^{b42}, OC(O)NR^{c42}R^{d42}, NR^{c42}R^{d42}, NR^{c42}C(O)R^{b42}, NR^{c42}C(O)OR^{a42}, NR^{c42}C(O)NR^{c42}R^{d42}, NR^{c42}S(O)₂R^{b42}, NR^{c42}S(O)₂NR^{c42}R^{d42}, S(O)₂R^{b42}, and S(O)₂NR^{c42}R^{d42};
each R^{a42}, R^{c42}, and R^{d42} is independently selected from H, C₁₋₆ alkyl, and C₁₋₆ haloalkyl;
each R^{b42} is independently selected from C₁₋₆ alkyl and C₁₋₆ haloalkyl;
each R⁵ is independently selected from oxo, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5}; wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{5A} substituents;
each R⁵¹ is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5}; wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{5A} substituents;
or, alternatively, any two R⁵ substituents on adjacent ring members, together with the atoms to which they are attached, form a 4-7 membered cycloalkyl or a 4-7 membered heterocycloalkyl ring, which are each optionally substituted by 1, 2, 3, or 4 independently selected R⁵¹ substituents;
each R^{a5}, R^{c5}, and R^{d5} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{5A} substituents;
each R^{b5} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{5A} substituents;
each R^{5A} is independently selected from D, halo, CN, C₁₋₆ alkyl, C₁₋₆ haloalkyl, C₃₋₅ cycloalkyl, OR^{a51}, SR^{a51}, C(O)R^{b51}, C(O)NR^{c51}R^{d51}, C(O)OR^{a51}, OC(O)R^{b51}, OC(O)NR^{c51}R^{d51}, NR^{c51}R^{d51}, NR^{c51}C(O)R^{b51}, NR^{c51}C(O)OR^{a51}, NR^{c51}C(O)NR^{c51}R^{d51}, NR^{c51}S(O)₂R^{b51}, NR^{c51}S(O)₂NR^{c51}R^{d51}, S(O)₂R^{b51}, and S(O)₂NR^{c51}R^{d51}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a51}, R^{c51}, and R^{d51} is independently selected from H, C₁₋₆ alkyl, and C₁₋₆ haloalkyl;
each R^{b51} is independently selected from C₁₋₆ alkyl and C₁₋₆ haloalkyl;
R⁶ is selected from H, D, CN, halo, C₁₋₃ alkyl, and C₁₋₃ haloalkyl; and
each R^{G} is independently selected from OH, CN, halo, C₁₋₃ alkyl, C₁₋₃ haloalkyl, cyano-C₁₋₃ alkyl, HO—C₁₋₃ alkyl, C₁₋₃ alkoxy-C₁₋₃ alkyl, C₃₋₄ cycloalkyl, C₁₋₃ alkoxy, C₁₋₃ haloalkoxy, amino, C₁₋₃ alkylamino, di(C₁₋₃ alkyl)amino, C₁₋₃ alkylsulfonyl, carbamyl, C₁₋₃ alkylcarbamyl, di(C₁₋₃ alkyl)carbamyl, C₁₋₃ alkylcarbonyl, C₁₋₃ alkoxycarbonyl, C₁₋₃ alkylcarbonyloxy, and C₁₋₃ alkylcarbonylamino.

In some embodiments:
n is 0, 1, or 2;
p is 0, 1, or 2;
W is N or CR¹;
Y is N or CR²;
Z is N or CR⁶;
X is O, S(O)₂, or N(-L-R⁴);
L is a bond, C₁₋₆ alkylene, C(O), C(O)O, S(O)₂, or S(O)₂NR^{a};
R^{a} is selected from H, C₁₋₆ alkyl, and C₁₋₆ haloalkyl;
or, alternatively, R^{a} and R⁴, taken together with the nitrogen atom to which they are attached, form a 4-7 membered heterocycloalkyl ring or 5-6 membered heteroaryl ring, each of which is optionally substituted by 1, 2, 3, or 4 independently selected R^{4A} substituents;
Ring moiety A is a pyrazole ring, an imidazole ring, a thiazole ring, an isothiazole ring, an oxazole ring, an isoxazole ring, a pyridine ring, or a pyrimidine ring;
R¹ is selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{b1}, SR^{b1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents.
each R^{a1}, R^{c1}, and R^{d1} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected RiA substituents;
each R^{b1} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1A} substituents;
each R^{1A} is independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NR^{c11}C(O)NR^{c}R^{a11}, NR^{c11}S(O)₂R^{b11}, NR^{c11}S(O)₂NR^{c11}R^{d11}, S(O)₂R^{b11}, and S(O)₂NR^{c11}R^{d11}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{1B} substituents;
each R^{b11} is independently selected from C₁₋₆ alkyl and C₁₋₆ haloalkyl, which are each optionally substituted with 1, 2, 3, or 4 independently selected R^{1B} substituents;
each R^{1B} is independently selected from OH, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₄ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
R² is selected from H, D, halo, CN, C₁₋₆ alkyl, and C₁₋₆ haloalkyl;
each R³ is independently selected from D, halo, CN, C₁₋₆ alkyl, C₁₋₆ haloalkyl, OR^{a3}, and NR^{c3}R^{d3} wherein said C₁₋₆ alkyl and C₁₋₆ haloalkyl are each optionally substituted with 1 or 2 independently selected R^{G} substituents;
each R^{a3}, R^{c3}, and R^{d3} is independently selected from H, C₁₋₆ alkyl, and C₁₋₆ haloalkyl;
each R^{b3} is independently selected from C₁₋₆ alkyl and C₁₋₆ haloalkyl;
R⁴ is selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{4A} substituents;
each R^{4A} is independently selected from oxo, halo, CN, C₁₋₆ alkyl, C₁₋₆ haloalkyl, C₃₋₇ cycloalkyl, phenyl, 4-7 membered heterocycloalkyl, 5-6 membered heteroaryl, OR^{a41}, SR^{a41}, C(O)R^{b41}, C(O)NR^{c41}R^{d41}, C(O)OR^{a41}, OC(O)R^{b41}, OC(O)NR^{c41}R^{d41}, NR^{c41}R^{d41}, NR^{c41}C(O)R^{b41}, NR^{c41}C(O)OR^{a41}, NR^{c41}C(O)NR^{c41}R^{d41}, NR^{c41}S(O)₂R^{b41}, NR^{c41}S(O)₂NR^{c41}R^{d41}, S(O)₂R^{b41}, and S(O)₂NR^{c41}R^{d41}, wherein said C₁₋₆ alkyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R^{4B} substituents;
each R^{a41}, R^{c41}, and R^{d41} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{4B} substituents;
each R^{b41} is independently selected from C₁₋₆ alkyl and C₁₋₆ haloalkyl, which are each optionally substituted with 1, 2, 3, or 4 independently selected R^{4B} substituents;
each R^{4B} is independently selected from halo, CN, C₁₋₆ alkyl, C₁₋₆ haloalkyl, OR^{a42}, C(O)R^{b42}, C(O)NR^{c42}R^{d42}, C(O)OR^{a42}, NR^{c42}R^{d42}, NR^{c42}C(O)R^{b42}, S(O)₂R^{b42}, and S(O)₂NR^{c42}R^{d42};
each R^{a42}, R^{c42}, and R^{d42} is independently selected from H, C₁₋₆ alkyl, and C₁₋₆ haloalkyl;
each R^{b42} is independently selected from C₁₋₆ alkyl and C₁₋₆ haloalkyl;
each R⁵ is independently selected from oxo, D, halo, CN, C₁₋₆ alkyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a5}, SR^{a5}, C(O)R^{b5}, C(O)NR^{c5}R^{d5}, C(O)OR^{a5}, OC(O)R^{b5}OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5}, wherein said C₁₋₆ alkyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{5A} substituents;
each R⁵¹ is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{5A} substituents;
or, alternatively, any two R⁵ substituents on adjacent ring members, together with the atoms to which they are attached, form a 4-7 membered cycloalkyl or a 4-7 membered heterocycloalkyl ring, which are each optionally substituted by 1, 2, 3, or 4 independently selected R⁵¹ substituents;
each R^{a5}, R^{c5}, and R^{d5} is independently selected from H, C₁₋₆ alkyl, C₁₋₆ haloalkyl, C₃₋₅ cycloalkyl, and 4-6 membered heterocycloalkyl, which are each optionally substituted with 1, 2, 3, or 4 independently selected R^{5A} substituents;
each R^{b5} is independently selected from C₁₋₆ alkyl and C₁₋₆ haloalkyl, which are each optionally substituted with 1, 2, 3, or 4 independently selected R^{5A} substituents;
each R^{5A} is independently selected from D, halo, CN, C₁₋₆ alkyl, C₁₋₆ haloalkyl, 4-7 membered heterocycloalkyl-C₁₋₄ alkyl, OR^{a51}, and NR^{c51}R^{d51};
each R^{a51}, R^{c51}, and R^{d51} is independently selected from H, C₁₋₆ alkyl, and C₁₋₆ haloalkyl;
R⁶ is selected from H, D, CN, halo, C₁₋₃ alkyl, and C₁₋₃ haloalkyl; and
each R^{G} is independently selected from OH, CN, halo, C₁₋₃ alkyl, C₁₋₃ haloalkyl, cyano-C₁₋₃ alkyl, HO—C₁₋₃ alkyl, C₁₋₃ alkoxy-C₁₋₃ alkyl, C₃₋₄ cycloalkyl, C₁₋₃ alkoxy, C₁₋₃ haloalkoxy, amino, C₁₋₃ alkylamino, di(C₁₋₃ alkyl)amino, C₁₋₃ alkylsulfonyl, carbamyl, C₁₋₃ alkylcarbamyl, di(C₁₋₃ alkyl)carbamyl, C₁₋₃ alkylcarbonyl, C₁₋₃ alkoxycarbonyl, C₁₋₃ alkylcarbonyloxy, and C₁₋₃ alkylcarbonylamino.

In some embodiments:
n is 0, 1, or 2;
p is 0, 1, or 2;
W is N or CR¹;
Y is N or CR²;
Z is N or CR⁶;
X is O, S(O)₂, or N(-L-R⁴);
L is a bond, C₁₋₆ alkylene, C(O), C(O)O, S(O)₂, or S(O)₂NR^{a};
R^{a} is selected from H, C₁₋₆ alkyl, and C₁₋₆ haloalkyl;
or, alternatively, R^{a} and R⁴, taken together with the nitrogen atom to which they are attached, form a 4-7 membered heterocycloalkyl ring or 5-6 membered heteroaryl ring, each of which is optionally substituted by 1, 2, 3, or 4 independently selected R^{4A} substituents;
Ring moiety A is a pyrazole ring, an imidazole ring, a thiazole ring, an isothiazole ring, an oxazole ring, an isoxazole ring, a pyridine ring, or a pyrimidine ring;
R¹ is selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{b1}, SR^{b1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents.
each R^{a1}, R^{c1}, and R^{d1} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
each R^{b1} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1A} substituents;
each R^{1A} is independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄alkyl, OR^{a11}, SR^{a11}, C(O)R^{b11}, C(O)NR^{c11}R^{d11}, C(O)OR^{a11}, OC(O)R^{b11}, OC(O)NR^{c11}R^{d11}, NR^{c11}R^{d11}, NR^{c11}C(O)R^{b11}, NR^{c11}C(O)OR^{a11}, NRCHC(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{1B} substituents;
each R^{b11} is independently selected from C₁₋₆ alkyl and C₁₋₆ haloalkyl, which are each optionally substituted with 1, 2, 3, or 4 independently selected R^{1B} substituents;
each R^{1B} is independently selected from OH, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₄ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
R² is selected from H, D, halo, CN, C₁₋₆ alkyl, and C₁₋₆ haloalkyl;
each R³ is independently selected from D, halo, CN, C₁₋₆ alkyl, C₁₋₆ haloalkyl, OR^{a3}, and NR^{c3}R^{d3} wherein said C₁₋₆ alkyl and C₁₋₆ haloalkyl are each optionally substituted with 1 or 2 independently selected R^{G} substituents;
each R^{a3}, R^{c3}, and R^{d3} is independently selected from H, C₁₋₆ alkyl, and C₁₋₆ haloalkyl;
each R^{b3} is independently selected from C₁₋₆ alkyl and C₁₋₆ haloalkyl;
R⁴ is selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl; wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{4A} substituents;
each R^{4A} is independently selected from oxo, halo, CN, C₁₋₆ alkyl, C₁₋₆ haloalkyl, C₃₋₇ cycloalkyl, phenyl, 4-7 membered heterocycloalkyl, 5-6 membered heteroaryl, OR^{a41}, SR^{a41}, C(O)R^{b41}, C(O)NR^{c41}R^{d41}, C(O)OR^{a41}, OC(O)R^{b41}, OC(O)NR^{c41}R^{d41}, NR^{c41}R^{d41}, NR^{c41}C(O)R^{b41}, NR^{c41}C(O)OR^{a41}, NR^{c41}C(O)NR^{c41}R^{d41}, NR^{c41}S(O)₂R^{b41}, NR^{c41}S(O)₂NR^{c41}R^{d41}, S(O)₂R^{b41}, and S(O)₂NR^{c41}R^{d41}, wherein said C₁₋₆ alkyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R^{4B} substituents;
each R^{a41}, R^{c41}, and R^{d41} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{4B} substituents;
each R^{b41} is independently selected from C₁₋₆ alkyl and C₁₋₆ haloalkyl, which are each optionally substituted with 1, 2, 3, or 4 independently selected R^{4B} substituents;
each R^{4B} is independently selected from halo, CN, C₁₋₆ alkyl, C₁₋₆ haloalkyl, OR^{a42}, C(O)R^{b42}, C(O)NR^{c42}R^{d42}, C(O)OR^{a42}, NR^{c42}R^{d42}, NR^{c42}C(O)R^{b42}, S(O)₂R^{b42}, and S(O)₂NR^{c42}R^{d42};
each R^{a42}, R^{c42}, and R^{d42} is independently selected from H, C₁₋₆ alkyl, and C₁₋₆ haloalkyl;
each R^{b42} is independently selected from C₁₋₆ alkyl and C₁₋₆ haloalkyl;
each R⁵ is independently selected from oxo, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5}; wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{5A} substituents;
each R⁵¹ is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5}; wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{5A} substituents;
or, alternatively, any two R⁵ substituents on adjacent ring members, together with the atoms to which they are attached, form a 4-7 membered cycloalkyl or a 4-7 membered heterocycloalkyl ring, which are each optionally substituted by 1, 2, 3, or 4 independently selected R⁵¹ substituents;
each R^{a5}, R^{c5}, and R^{d5} is independently selected from H, C₁₋₆ alkyl, C₁₋₆ haloalkyl, C₃₋₅ cycloalkyl, and 4-6 membered heterocycloalkyl are each optionally substituted with 1, 2, 3, or 4 independently selected R^{5A} substituents;
each R^{b5} is independently selected from C₁₋₆ alkyl and C₁₋₆ haloalkyl, which are each optionally substituted with 1, 2, 3, or 4 independently selected R^{5A} substituents;
each R^{5A} is independently selected from D, halo, CN, C₁₋₆ alkyl, C₁₋₆ haloalkyl, OR^{a51} and NR^{c51}R^{d51}; and
each R^{a51}, R^{c51}, and R^{d51} is independently selected from H, C₁₋₆ alkyl, and C₁₋₆ haloalkyl;
R⁶ is selected from H, D, CN, halo, C₁₋₃ alkyl, and C₁₋₃ haloalkyl; and
each R^{G} is independently selected from OH, CN, halo, C₁₋₃ alkyl, C₁₋₃ haloalkyl, cyano-C₁₋₃ alkyl, HO—C₁₋₃ alkyl, C₁₋₃ alkoxy-C₁₋₃ alkyl, C₃₋₄ cycloalkyl, C₁₋₃ alkoxy, C₁₋₃ haloalkoxy, amino, C₁₋₃ alkylamino, di(C₁₋₃ alkyl)amino, C₁₋₃ alkylsulfonyl, carbamyl, C₁₋₃ alkylcarbamyl, di(C₁₋₃ alkyl)carbamyl, C₁₋₃ alkylcarbonyl, C₁₋₃ alkoxycarbonyl, C₁₋₃ alkylcarbonyloxy, and C₁₋₃ alkylcarbonylamino.

In some embodiments, the compound is a compound of Formula (Ia):

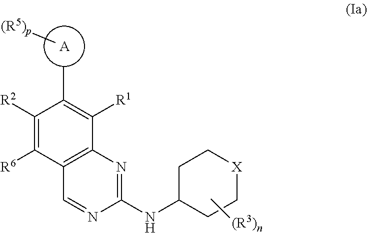

or a pharmaceutically acceptable salt thereof.

In some embodiments, the compound is a compound of Formula (Ib):

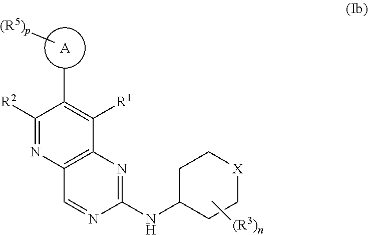

or a pharmaceutically acceptable salt thereof.

In some embodiments, the compound is a compound of Formula (Ic):

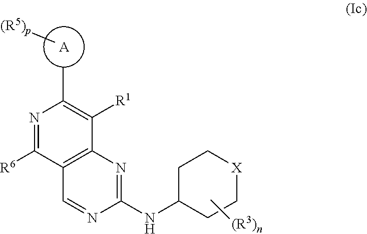

or a pharmaceutically acceptable salt thereof.

In some embodiments, the compound is selected from:
(3R,4R)-4-((7-(1H-Pyrazol-4-yl)quinazolin-2-yl)amino)-1 (methylsulfonyl)piperidin-3-ol;
(3R,4R)-4-((7-(1H-Pyrazol-4-yl)pyrido[3,2-d]pyrimidin-2-yl)amino)-1-(methylsulfonyl)piperidin-3-ol;
(3R,4R)-4-((7-(3-Cyclopropyl-1H-pyrazol-4-yl)pyrido[4,3-d]pyrimidin-2-yl)amino)-1-(methylsulfonyl)piperidin-3-ol;
(3R,4R)-4-((6-Chloro-7-(1H-pyrazol-4-yl)quinazolin-2-yl)amino)-1-(methylsulfonyl)piperidin-3-ol;
(3R,4R)-4-((7-(3-Methyl-1H-pyrazol-4-yl)quinazolin-2-yl)amino)-1-(methylsulfonyl)piperidin-3-ol;
4-(2-(((3R,4R)-3-Hydroxy-1-(methylsulfonyl)piperidin-4-yl)amino)quinazolin-7-yl)pyridin-2(1H)-one;
(3R,4R)-4-((7-(1H-Imidazol-4-yl)quinazolin-2-yl)amino)-1-(methylsulfonyl)piperidin-3-ol;
(3R,4R)-4-((7-(1H-Pyrazol-4-yl)quinazolin-2-yl)amino)-1-((1-methyl-1H-pyrazol-4-yl)sulfonyl)piperidin-3-ol;
(3R,4R)-4-((7-(3-Methyl-1H-pyrazol-4-yl)quinazolin-2-yl)amino)-1-((1-methyl-1H-pyrazol-4-yl)sulfonyl)piperidin-3-ol;
(3R,4R)-1-(Cyclopropylsulfonyl)-4-((7-(3-methyl-1H-pyrazol-4-yl)quinazolin-2-yl)amino)piperidin-3-ol;
(3R,4R)-1-((3-Methoxy-1-methyl-1H-pyrazol-4-yl)sulfonyl)-4-((7-(3-methyl-1H-pyrazol-4-yl)quinazolin-2-yl)amino)piperidin-3-ol;
Methyl (3R,4R)-3-hydroxy-4-((7-(3-methyl-1H-pyrazol-4-yl)quinazolin-2-yl)amino)piperidine-1-carboxylate;
(3R,4R)-4-((7-(1H-Pyrazol-4-yl)quinazolin-2-yl)amino)-1-(cyclopropylsulfonyl)piperidin-3-ol;
1-((3R,4R)-3-hydroxy-4-((7-(3-methyl-1H-pyrazol-4-yl)quinazolin-2-yl)amino)piperidin-1-yl)ethan-1-one;
(3R,4R)-4-((7-(3-Cyclopropyl-1H-pyrazol-4-yl)quinazolin-2-yl)amino)-1-(pyridin-3-ylmethyl)piperidin-3-ol;
(3R,4R)-4-((7-(3-(2,2-Difluoroethoxy)-1H-pyrazol-4-yl)quinazolin-2-yl)amino)-1-(methylsulfonyl)piperidin-3-ol;
(3S,4R)-4-((8-(3-Chloro-4-((dimethylamino)methyl)phenyl)-7-(1H-pyrazol-4-yl)quinazolin-2-yl)amino)tetrahydro-2H-pyran-3-ol;
(3S,4R)-4-((8-(4-Methylpiperazin-1-yl)-7-(1H-pyrazol-4-yl)quinazolin-2-yl)amino)tetrahydro-2H-pyran-3-ol;
((3R,4R)-4-((8-Isopropoxy-7-(1-methyl-1H-pyrazol-4-yl)quinazolin-2-yl)amino)-1-(methylsulfonyl)piperidin-3-yl)methanol;
(3R,4R)-1-(Methylsulfonyl)-4-((7-(pyridin-3-yl)quinazolin-2-yl)amino)piperidin-3-ol;
(3R,4R)-1-(Methylsulfonyl)-4-((7-(thiazol-5-yl)quinazolin-2-yl)amino)piperidin-3-ol;
(3R,4R)-4-((7-(6,7-Dihydro-5H-pyrazolo[5,1-b][1,3]oxazin-3-yl)quinazolin-2-yl)amino)-1-(methylsulfonyl)piperidin-3-ol;
(3R,4R)-1-((3,3-Difluoroazetidin-1-yl)sulfonyl)-4-((7-(3-methyl-1H-pyrazol-4-yl)quinazolin-2-yl)amino)piperidin-3-ol;
(3R,4R)-4-((7-(3-Cyclopropyl-1H-pyrazol-4-yl)-6,8-difluoroquinazolin-2-yl)amino)-1-(methylsulfonyl)piperidin-3-ol;
2-((3R,4R)-4-((7-(3-Cyclopropyl-1H-pyrazol-4-yl)quinazolin-2-yl)amino)-3-hydroxypiperidin-1-yl)acetamide; and
(S)-5-(((3R,4R)-4-((7-(3-Cyclopropyl-1H-pyrazol-4-yl)quinazolin-2-yl)amino)-3-hydroxypiperidin-1-yl)methyl)pyrrolidin-2-one;
or a pharmaceutically acceptable salt thereof.

In some embodiments, the compound is selected from:
(3R,4R)-1-((1-cyclopropyl-1H-pyrazol-3-yl)sulfonyl)-4-((7-(3-methyl-1H-pyrazol-4-yl)quinazolin-2-yl)amino)piperidin-3-ol;
methyl (3R,4R)-4-((7-(3-cyclopropyl-1H-pyrazol-4-yl)quinazolin-2-yl)amino)-3-hydroxypiperidine-1-carboxylate;
methyl (3R,4R)-4-((7-(3-ethyl-1H-pyrazol-4-yl)quinazolin-2-yl)amino)-3-hydroxypiperidine-1-carboxylate;
1-((3R,4R)-4-((7-(3-cyclopropyl-1H-pyrazol-4-yl)quinazolin-2-yl)amino)-3-hydroxypiperidin-1-yl)ethan-1-one;
(3S,4R)-4-((7-(1H-pyrazol-4-yl)quinazolin-2-yl)amino)tetrahydro-2H-pyran-3-ol;
(3S,4R)-4-((7-(1H-pyrazol-4-yl)quinazolin-2-yl)amino)-1-(methylsulfonyl)piperidin-3-ol;
rac-((3R,4R)-4-((7-(1H-pyrazol-4-yl)quinazolin-2-yl)amino)-1-(methylsulfonyl)piperidin-3-yl)methanol;
(3R,4R)-4-((7-(3-isopropyl-1H-pyrazol-4-yl)quinazolin-2-yl)amino)-1-(methylsulfonyl)piperidin-3-ol;
(3R,4R)-4-((7-(3-cyclopropyl-1H-pyrazol-4-yl)quinazolin-2-yl)amino)-1-(methylsulfonyl)piperidin-3-ol;
(3R,4R)-4-((7-(3-fluoro-1H-pyrazol-4-yl)quinazolin-2-yl)amino)-1-(methylsulfonyl)piperidin-3-ol;
(3R,4R)-4-((7-(3-methoxy-1H-pyrazol-4-yl)quinazolin-2-yl)amino)-1-(methylsulfonyl)piperidin-3-ol;
(3R,4R)-4-((7-(1-methyl-1H-imidazol-4-yl)quinazolin-2-yl)amino)-1-(methylsulfonyl)piperidin-3-ol;
(3R,4R)-4-((7-(1-methyl-1H-imidazol-5-yl)quinazolin-2-yl)amino)-1-(methylsulfonyl)piperidin-3-ol;
(3R,4R)-1-(methylsulfonyl)-4-((7-(pyridin-4-yl)quinazolin-2-yl)amino)piperidin-3-ol;
(3R,4R)-1-(methylsulfonyl)-4-((7-(pyridazin-4-yl)quinazolin-2-yl)amino)piperidin-3-ol;
(3R,4R)-4-((7-(isothiazol-4-yl)quinazolin-2-yl)amino)-1-(methylsulfonyl)piperidin-3-ol;
(3R,4R)-1-(methylsulfonyl)-4-((7-(3-(trifluoromethyl)-1H-pyrazol-4-yl)quinazolin-2-yl)amino)piperidin-3-ol;
4-(2-(((3R,4R)-3-hydroxy-1-(methylsulfonyl)piperidin-4-yl)amino)quinazolin-7-yl)-1H-pyrazole-3-carbonitrile;
(3R,4R)-4-((7-(3-ethyl-1H-pyrazol-4-yl)quinazolin-2-yl)amino)-1-(methylsulfonyl)piperidin-3-ol;
(3R,4R)-4-((7-(1-methyl-1H-pyrazol-5-yl)quinazolin-2-yl)amino)-1-(methylsulfonyl)piperidin-3-ol;
(3R,4R)-4-((7-(1H-pyrazol-5-yl)quinazolin-2-yl)amino)-1-(methylsulfonyl)piperidin-3-ol;
(3R,4R)-4-((7-(1-cyclopropyl-1H-pyrazol-5-yl)quinazolin-2-yl)amino)-1-(methylsulfonyl)piperidin-3-ol;
(3R,4R)-4-((7-(1-ethyl-1H-pyrazol-5-yl)quinazolin-2-yl)amino)-1-(methylsulfonyl)piperidin-3-ol;
(3R,4R)-4-((7-(1-ethyl-1H-imidazol-5-yl)quinazolin-2-yl)amino)-1-(methylsulfonyl)piperidin-3-ol;
(3R,4R)-4-((7-(1-cyclopropyl-1H-imidazol-5-yl)quinazolin-2-yl)amino)-1-(methylsulfonyl)piperidin-3-ol;
(3R,4R)-4-((7-(3-fluoro-1-methyl-1H-pyrazol-4-yl)quinazolin-2-yl)amino)-1-(methylsulfonyl)piperidin-3-ol;
(3R,4R)-4-((7-(5-fluoro-1-methyl-1H-pyrazol-4-yl)quinazolin-2-yl)amino)-1-(methylsulfonyl)piperidin-3-ol;
(3R,4R)-1-(methylsulfonyl)-4-((7-(3-(tetrahydro-2H-pyran-4-yl)-1H-pyrazol-4-yl)quinazolin-2-yl)amino)piperidin-3-ol;
(3R,4R)-1-(methylsulfonyl)-4-((7-(3-morpholino-1H-pyrazol-4-yl)quinazolin-2-yl)amino)piperidin-3-ol;
(3R,4R)-4-((7-(3-((dimethylamino)methyl)-1H-pyrazol-4-yl)quinazolin-2-yl)amino)-1-(methylsulfonyl)piperidin-3-ol;
methyl (3R,4R)-4-((7-(3-(cyclopropylmethyl)-1H-pyrazol-4-yl)quinazolin-2-yl)amino)-3-hydroxypiperidine-1-carboxylate;
methyl (3R,4R)-4-((7-(1-cyclopropyl-1H-imidazol-5-yl)quinazolin-2-yl)amino)-3-hydroxypiperidine-1-carboxylate;
methyl (3R,4R)-4-((7-(1-ethyl-1H-imidazol-5-yl)quinazolin-2-yl)amino)-3-hydroxypiperidine-1-carboxylate;
methyl (3R,4R)-3-hydroxy-4-((7-(3-morpholino-1H-pyrazol-4-yl)quinazolin-2-yl)amino)piperidine-1-carboxylate;
methyl (3R,4R)-3-hydroxy-4-((7-(3-(tetrahydro-2H-pyran-4-yl)-1H-pyrazol-4-yl)quinazolin-2-yl)amino)piperidine-1-carboxylate;
methyl (3R,4R)-3-hydroxy-4-((7-(3-(2-hydroxyethyl)-1H-pyrazol-4-yl)quinazolin-2-yl)amino)piperidine-1-carboxylate;
methyl (3R,4R)-4-((7-(3-(4-(azetidin-1-ylmethyl)-3-fluorophenyl)-1H-pyrazol-4-yl)quinazolin-2-yl)amino)-3-hydroxypiperidine-1-carboxylate;
methyl (3R,4R)-4-((7-(3-(4-((dimethylamino)methyl)-3-fluorophenyl)-1H-pyrazol-4-yl)quinazolin-2-yl)amino)-3-hydroxypiperidine-1-carboxylate;
(3R,4R)-3-hydroxy-N,N-dimethyl-4-((7-(3-methyl-1H-pyrazol-4-yl)quinazolin-2-yl)amino)piperidine-1-sulfonamide;
(3R,4R)-4-((8-fluoro-7-(3-methyl-1H-pyrazol-4-yl)quinazolin-2-yl)amino)-1-(methylsulfonyl)piperidin-3-ol;
N-((3R,4R)-3-methoxy-1-(methylsulfonyl)piperidin-4-yl)-7-(3-methyl-1H-pyrazol-4-yl)quinazolin-2-amine;
(3R,4R)-1-(cyclopropylsulfonyl)-4-((7-(3-ethyl-1H-pyrazol-4-yl)-6,8-difluoroquinazolin-2-yl)amino)piperidin-3-ol;
(3R,4R)-4-((7-(3-methyl-1H-pyrazol-4-yl)pyrido[2,3-d]pyrimidin-2-yl)amino)-1-(methylsulfonyl)piperidin-3-ol;
(3R,4R)-4-((7-(1H-pyrazol-4-yl)pyrido[4,3-d]pyrimidin-2-yl)amino)-1-(methylsulfonyl)piperidin-3-ol;
(3R,4R)-4-((7-(3-methyl-1H-pyrazol-4-yl)pyrido[4,3-d]pyrimidin-2-yl)amino)-1-(methylsulfonyl)piperidin-3-ol;
(3R,4R)-4-((7-(3-isopropyl-1H-pyrazol-4-yl)pyrido[4,3-d]pyrimidin-2-yl)amino)-1-(methylsulfonyl)piperidin-3-ol;
(3R,4R)-4-((7-(1-(2,2-difluoroethyl)-1H-imidazol-5-yl)quinazolin-2-yl)amino)-1-(methylsulfonyl)piperidin-3-ol;
(3R,4R)-4-((7-(3-(1-methyl-1,2,3,6-tetrahydropyridin-4-yl)-1H-pyrazol-4-yl)quinazolin-2-yl)amino)-1-(methylsulfonyl)piperidin-3-ol;
(3R,4R)-4-((7-(3-(1-methylpiperidin-4-yl)-1H-pyrazol-4-yl)quinazolin-2-yl)amino)-1-(methylsulfonyl)piperidin-3-ol;
(3R,4R)-4-((7-(3-(2-methoxypyridin-4-yl)-1H-pyrazol-4-yl)quinazolin-2-yl)amino)-1-(methylsulfonyl)piperidin-3-ol;
(3R,4R)-4-((7-(5-(3-methoxyprop-1-yn-1-yl)-1H-pyrazol-4-yl)quinazolin-2-yl)amino)-1-(methylsulfonyl)piperidin-3-ol;
(3R,4R)-1-(cyclopropylsulfonyl)-4-((6,8-difluoro-7-(3-methyl-1H-pyrazol-4-yl)quinazolin-2-yl)amino)piperidin-3-ol;
(3R,4R)-4-((7-(3-ethyl-1H-pyrazol-4-yl)-6,8-difluoroquinazolin-2-yl)amino)-1-(methylsulfonyl)piperidin-3-ol;
(3R,4R)-4-((6,8-difluoro-7-(3-methyl-1H-pyrazol-4-yl)quinazolin-2-yl)amino)-1-(methylsulfonyl)piperidin-3-ol;
(3R,4R)-4-((6,8-difluoro-7-(1-methyl-1H-imidazol-5-yl)quinazolin-2-yl)amino)-1-(methylsulfonyl)piperidin-3-ol;
(3R,4R)-4-((6-fluoro-7-(3-methyl-1H-pyrazol-4-yl)quinazolin-2-yl)amino)-1-(methylsulfonyl)piperidin-3-ol;
(3R,4R)-4-((6-fluoro-7-(1H-pyrazol-4-yl)quinazolin-2-yl)amino)-1-(methylsulfonyl)piperidin-3-ol;
(3R,4R)-4-((7-(3-cyclopropyl-1H-pyrazol-4-yl)-6-fluoroquinazolin-2-yl)amino)-1-(methylsulfonyl)piperidin-3-ol;
(3R,4R)-4-((7-(3-(difluoromethyl)-1H-pyrazol-4-yl)-6-fluoroquinazolin-2-yl)amino)-1-(methylsulfonyl)piperidin-3-ol
(3R,4R)-4-((6-fluoro-7-(3-fluoro-1H-pyrazol-4-yl)quinazolin-2-yl)amino)-1-(methylsulfonyl)piperidin-3-ol;
(3R,4R)-4-((6-fluoro-7-(1-methyl-1H-imidazol-5-yl)quinazolin-2-yl)amino)-1-(methylsulfonyl)piperidin-3-ol;
(3R,4R)-1-(cyclopropylsulfonyl)-4-((6-fluoro-7-(1-methyl-1H-imidazol-5-yl)quinazolin-2-yl)amino)piperidin-3-ol;
(3R,4R)-4-((6-fluoro-7-(1-methyl-1H-imidazol-5-yl)quinazolin-2-yl)amino)-3-hydroxypiperidin-1-yl)(1-(trifluoromethyl)cyclopropyl)methanone;
(1-(difluoromethyl)cyclopropyl)((3R,4R)-4-((6-fluoro-7-(1-methyl-1H-imidazol-5-yl)quinazolin-2-yl)amino)-3-hydroxypiperidin-1-yl)methanone;
((3R,4R)-4-((6-fluoro-7-(1-methyl-1H-imidazol-5-yl)quinazolin-2-yl)amino)-3-hydroxypiperidin-1-yl)(1-methylcyclopropyl)methanone;
(3R,4R)-1-(cyclopropylsulfonyl)-4-((6-fluoro-7-(1-methyl-1H-pyrazol-5-yl)quinazolin-2-yl)amino)piperidin-3-ol;
(3R,4R)-4-((5-fluoro-7-(1-methyl-1H-imidazol-5-yl)quinazolin-2-yl)amino)-1-(methylsulfonyl)piperidin-3-ol;
(3R,4R)-4-((5-fluoro-7-(3-methyl-1H-pyrazol-4-yl)quinazolin-2-yl)amino)-1-(methylsulfonyl)piperidin-3-ol;
(3R,4R)-4-((5-fluoro-7-(1H-pyrazol-4-yl)quinazolin-2-yl)amino)-1-(methylsulfonyl)piperidin-3-ol;
(3R,4R)-4-((6-fluoro-7-(1-(2-(methylamino)ethyl)-1H-pyrazol-4-yl)quinazolin-2-yl)amino)-1-(methylsulfonyl)piperidin-3-ol;
(3R,4R)-4-((6,8-difluoro-7-(1-(2-(methylamino)ethyl)-1H-pyrazol-4-yl)quinazolin-2-yl)amino)-1-(methylsulfonyl)piperidin-3-ol;
(3R,4R)-4-((7-(1-(2-(methylamino)ethyl)-1H-pyrazol-4-yl)quinazolin-2-yl)amino)-1-(methylsulfonyl)piperidin-3-ol;
((3R,4R)-4-((6-fluoro-7-(1-(2-(methylamino)ethyl)-1H-pyrazol-4-yl)quinazolin-2-yl)amino)-3-hydroxypiperidin-1-yl)(1-methylcyclopropyl)methanone;
(3,3-difluorocyclobutyl)((3R,4R)-4-((6-fluoro-7-(1-(2-(methylamino)ethyl)-1H-pyrazol-4-yl)quinazolin-2-yl)amino)-3-hydroxypiperidin-1-yl)methanone;
2,2-difluoroethyl (3R,4R)-4-((6-fluoro-7-(1-(2-(methylamino)ethyl)-1H-pyrazol-4-yl)quinazolin-2-yl)amino)-3-hydroxypiperidine-1-carboxylate;
((3R,4R)-4-((6-fluoro-7-(1-(2-(methylamino)ethyl)-1H-pyrazol-4-yl)quinazolin-2-yl)amino)-3-hydroxypiperidin-1-yl)(1-(trifluoromethyl)cyclobutyl)methanone;
(1-(difluoromethyl)cyclopropyl)((3R,4R)-4-((6-fluoro-7-(1-(2-(methylamino)ethyl)-1H-pyrazol-4-yl)quinazolin-2-yl)amino)-3-hydroxypiperidin-1-yl)methanone;
methyl (3R,4R)-4-((6-fluoro-7-(1H-pyrazol-4-yl)quinazolin-2-yl)amino)-3-hydroxypiperidine-1-carboxylate;
methyl (3R,4R)-4-((7-(3-ethyl-1H-pyrazol-4-yl)-6-fluoroquinazolin-2-yl)amino)-3-hydroxypiperidine-1-carboxylate; and
methyl (3R,4R)-4-((6-fluoro-7-(3-methyl-1H-pyrazol-4-yl)quinazolin-2-yl)amino)-3-hydroxypiperidine-1-carboxylate;
or a pharmaceutically acceptable salt thereof.

In some embodiments, 1, 2, 3, 4, 5, 6, 7, or 8 hydrogen atoms, attached to carbon atoms of “alkyl”, “alkenyl”, “alkynyl”, “aryl”, “phenyl”, “cycloalkyl”, “heterocycloalkyl”, or “heteroaryl” substituents or “—C₁₋₄ alkyl-” and “alkylene” linking groups, as described herein, are optionally replaced by deuterium atoms.

It is further appreciated that certain features of the invention, which are, for clarity, described in the context of separate embodiments, can also be provided in combination in a single embodiment as if the embodiments were claims written in multiple dependent form. Conversely, various features of the invention which are, for brevity, described in the context of a single embodiment, can also be provided separately or in any suitable subcombination.

At various places in the present specification, divalent linking substituents are described. Unless otherwise specified, it is specifically intended that each divalent linking substituent include both the forward and backward forms of the linking substituent. For example, —NR(CR′R″)ₙ— includes both —NR(CR′R″)ₙ— and —(CR′R″)ₙNR—. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency, that the designated atom's normal valency is not exceeded, and that the substitution results in a stable compound.

As used herein, the term “independently selected from” means that each occurrence of a variable or substituent are independently selected at each occurrence from the applicable list.

As used herein, the phrase “each ‘variable’ is independently selected from” means substantially the same as wherein “at each occurrence ‘variable’ is selected from.”

When any variable (e.g., R^{G}) occurs more than one time in any constituent or formula for a compound, its definition at each occurrence is independent of its definition at every other occurrence. Thus, for example, if a group is shown to be substituted with 1, 2, 3, or 4 independently selected R^{G} substituents, then said group may optionally be substituted with up to four R^{G} groups and R^{G} at each occurrence is selected independently from the definition of R^{G}.

In some embodiments, substituents are indicated as floating substituents—e.g., (R³)ₙ or (R⁵)ₚ in Formula (I):

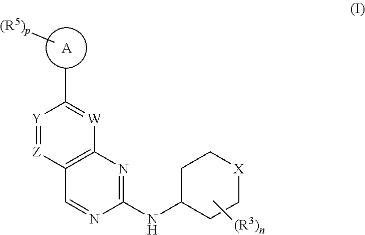

It is understood that substituent R³ can occur n number of times on the ring, and R³ can be a different moiety at each occurrence. It is to be understood that each R group may replace any hydrogen atom attached to a ring atom, except that R³ may not replace -L-R⁴ in the variable X. (R⁵)ₚ is another example of a floating substituent.

Throughout the definitions, the term “Cn-m” indicates a range which includes the endpoints, wherein n and m are integers and indicate the number of carbons. Examples include C₁₋₃, C₁₋₄, C₁₋₆, and the like.

As used herein, the term “Cn-m alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (i-Pr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms. As used herein, the term “Cₙ₋ₘ alkoxy”, employed alone or in combination with other terms, refers to a group of formula-O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, the term “amino” refers to a group of formula —NH₂.

As used herein, the term “aryl,” employed alone or in combination with other terms, refers to an aromatic hydrocarbon group, which may be monocyclic or polycyclic (e.g., having 2 fused rings). The term “Cₙ₋ₘ aryl” refers to an aryl group having from n to m ring carbon atoms. In some embodiments, the aryl group has 6 to 10 carbon atoms. In some embodiments, the aryl group is phenyl or naphthyl. In some embodiments, the aryl is phenyl.

As used herein, “halo” refers to F, Cl, Br, or I. In some embodiments, halo is F, Cl, or Br. In some embodiments, halo is F or Cl. In some embodiments, halo is F. In some embodiments, halo is Cl.

As used herein, “Cₙ₋ₘ haloalkoxy” refers to a group of formula —O-haloalkyl having n to m carbon atoms. Example haloalkoxy groups include OCF₃ and OCHF₂. In some embodiments, the haloalkoxy group is fluorinated only. In some embodiments, the alkyl group has 1 to 6, 1 to 4, or 1 to 3 carbon atoms.

As used herein, the term “Cₙ₋ₘ haloalkyl,” employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group of the haloalkyl has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like.

As used herein, the term “thio” refers to a group of formula —SH.

As used herein, the term “Cₙ₋ₘ alkylamino” refers to a group of formula —NH(alkyl), wherein the alkyl group has n to m carbon atoms. In some embodiments, the alkyl group of the alkylamino has 1 to 6, 1 to 4, or 1 to 3 carbon atoms.

As used herein, the term “Cₙ₋ₘ alkoxycarbonyl” refers to a group of formula —C(O)O— alkyl, wherein the alkyl group has n to m carbon atoms. In some embodiments, the alkyl group of the alkoxycarbonyl has 1 to 6, 1 to 4, or 1 to 3 carbon atoms.

As used herein, the term “Cₙ₋ₘ alkylcarbonyl” refers to a group of formula —C(O)-alkyl, wherein the alkyl group has n to m carbon atoms. In some embodiments, the alkyl group of the alkylcarbonyl has 1 to 6, 1 to 4, or 1 to 3 carbon atoms.

As used herein, the term “Cₙ₋ₘ alkylcarbonylamino” refers to a group of formula —NHC(O)-alkyl, wherein the alkyl group has n to m carbon atoms. In some embodiments, the alkyl group of the alkylcarbonylamino has 1 to 6, 1 to 4, or 1 to 3 carbon atoms.

As used herein, the term “Cₙ₋ₘ alkoxycarbonylamino” refers to a group of formula —NHC(O)O(Cₙ₋ₘ alkyl), wherein the alkyl group has n to m carbon atoms. In some embodiments, the alkyl group of the alkoxycarbonylamino has 1 to 6, 1 to 4, or 1 to 3 carbon atoms.

As used herein, the term “Cₙ₋ₘ alkylsulfonylamino” refers to a group of formula —NHS(O)₂-alkyl, wherein the alkyl group has n to m carbon atoms. In some embodiments, the alkyl group of the alkylsulfonylamino has 1 to 6, 1 to 4, or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O)₂NH(alkyl), wherein the alkyl group has n to m carbon atoms. In some embodiments, the alkyl group of the alkylaminosulfonyl has 1 to 6, 1 to 4, or 1 to 3 carbon atoms.

As used herein, the term “di(Cₙ₋ₘ alkyl)aminosulfonyl” refers to a group of formula —S(O)₂N(alkyl)₂, wherein each alkyl group independently has n to m carbon atoms. In some embodiments, each alkyl group of the dialkylaminosulfonyl has, independently, 1 to 6, 1 to 4, or 1 to 3 carbon atoms.

As used herein, the term “aminosulfonylamino” refers to a group of formula —NHS(O)₂NH₂.

As used herein, the term “Cₙ₋ₘ alkylaminosulfonylamino” refers to a group of formula —NHS(O)₂NH(alkyl), wherein the alkyl group has n to m carbon atoms. In some embodiments, the alkyl group of the alkylaminosulfonylamino has 1 to 6, 1 to 4, or 1 to 3 carbon atoms.

As used herein, the term “di(Cₙ₋ₘ alkyl)aminosulfonylamino” refers to a group of formula —NHS(O)₂N(alkyl)₂, wherein each alkyl group independently has n to m carbon atoms. In some embodiments, each alkyl group of the dialkylaminosulfonylamino has, independently, 1 to 6, 1 to 4, or 1 to 3 carbon atoms.

As used herein, the term “aminocarbonylamino,” employed alone or in combination with other terms, refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group of the alkylaminocarbonylamino has 1 to 6, 1 to 4, or 1 to 3 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group of the dialkylaminocarbonylamino has, independently, 1 to 6, 1 to 4, or 1 to 3 carbon atoms.

As used herein, the term “Cₙ₋ₘ alkylcarbamyl” refers to a group of formula —C(O)—NH(alkyl), wherein the alkyl group has n to m carbon atoms. In some embodiments, the alkyl group of the alkylcarbamyl has 1 to 6, 1 to 4, or 1 to 3 carbon atoms.

As used herein, the term “Cₙ₋ₘ alkylthio” refers to a group of formula —S-alkyl, wherein the alkyl group has n to m carbon atoms. In some embodiments, the alkyl group of the alkylthio has 1 to 6, 1 to 4, or 1 to 3 carbon atoms.

As used herein, the term “Cₙ₋ₘ alkylsulfinyl” refers to a group of formula —S(O)-alkyl, wherein the alkyl group has n to m carbon atoms. In some embodiments, the alkyl group of the alkylsulfinyl has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group of the alkylsulfonyl has 1 to 6, 1 to 4, or 1 to 3 carbon atoms.

As used herein, the term “cyano-Cₙ₋ₘ alkyl” refers to a group of formula —(Cₙ₋ₘ alkylene)-CN, wherein the alkylene group has n to m carbon atoms. As used herein, the term “cyano-C₁₋₆ alkyl” refers to a group of formula —(C₁₋₆ alkylene)-CN. As used herein, the term “cyano-C₁₋₃ alkyl” refers to a group of formula —(C₁₋₃ alkylene)-CN.

As used herein, the term “HO—Cₙ₋ₘ alkyl” refers to a group of formula —(Cₙ₋ₘ alkylene)-OH, wherein the alkylene group has n to m carbon atoms. As used herein, the term “HO—C₁₋₃ alkyl” refers to a group of formula —(C₁₋₃ alkylene)-OH.

As used herein, the term “Cₙ₋ₘ alkoxy-Cₒ₋ₚ alkyl” refers to a group of formula —(Cₙ₋ₘ alkylene)-O(Cₒ₋ₚ alkyl), wherein the alkylene group has n to m carbon atoms and the alkyl group has o to p carbon atoms. As used herein, the term “C₁₋₆ alkoxy-C₁₋₆ alkyl” refers to a group of formula —(C₁₋₆ alkylene)-O(C₁₋₆ alkyl). As used herein, the term “C₁₋₃ alkoxy-C₁₋₃ alkyl” refers to a group of formula —(C₁₋₃ alkylene)-O(C₁₋₃ alkyl).

As used herein, the term “carboxy” refers to a group of formula —C(O)OH.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group of the dialkylamino independently has 1 to 6, 1 to 4, or 1 to 3 carbon atoms.

As used herein, the term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group of the dialkylcarbamyl independently has 1 to 6, 1 to 4, or 1 to 3 carbon atoms.

As used herein, the term “Cₙ₋ₘ alkylcarbonyloxy” is a group of formula —OC(O)-alkyl, wherein the alkyl group has n to m carbon atoms. In some embodiments, the alkyl group of the alkylcarbonyloxy has 1 to 6, 1 to 4, or 1 to 3 carbon atoms.

As used herein, “aminocarbonyloxy” is a group of formula —OC(O)—NH₂.

As used herein, “Cₙ₋ₘ alkylaminocarbonyloxy” is a group of formula —OC(O)—NH-alkyl, wherein the alkyl group has n to m carbon atoms. In some embodiments, the alkyl group of the alkylaminocarbonyloxy has 1 to 6, 1 to 4, or 1 to 3 carbon atoms.

As used herein, “di(Cₙ₋ₘalkyl)aminocarbonyloxy” is a group of formula —OC(O)—N(alkyl)₂, wherein each alkyl group has, independently, n to m carbon atoms. In some embodiments, each alkyl group of the dialkylaminocarbonyloxy independently has 1 to 6, 1 to 4, or 1 to 3 carbon atoms.

As used herein “Cₙ₋ₘ alkoxycarbonylamino” refers to a group of formula —NHC(O)—O— alkyl, wherein the alkyl group has n to m carbon atoms.

As used herein, the term “carbamyl” to a group of formula —C(O)NH₂.

As used herein, the term “carbonyl,” employed alone or in combination with other terms, refers to a —C(O)— group.

As used herein, “cycloalkyl” refers to non-aromatic cyclic hydrocarbons including cyclized alkyl and alkenyl groups. Cycloalkyl groups can include mono- or polycyclic (e.g., having 2, 3 or 4 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C₄₋₁₀ spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, and the like. In some embodiments, cycloalkyl is cyclopropyl, cyclobutyl, cyclopentyl, or cyclohexyl.

As used herein, “heteroaryl” refers to a monocyclic or polycyclic (e.g., having 2, 3, or 4 fused rings) aromatic heterocycle having at least one heteroatom ring member selected from N, O, or S. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, and S. In some embodiments, the heteroaryl is a 5-6 monocyclic heteroaryl having 1 or 2 heteroatom ring members independently selected from N, O, and S. In some embodiments, the heteroaryl group contains 5 to 10 or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, pyridyl, pyrimidinyl, pyrazinyl, pyridazinyl, pyrrolyl, pyrazolyl, azolyl, oxazolyl, isoxazolyl, thiazolyl, isothiazolyl, imidazolyl, furyl, thienyl, triazolyl (e.g., 1,2,3-triazolyl, 1,2,4-triazolyl, 1,3,4-triazolyl), tetrazolyl, thiadiazolyl (e.g., 1,2,3-thiadiazolyl, 1,2,4-thiadiazolyl, 1,3,4-thiadiazolyl), quinolinyl, isoquinolinyl, indolyl, benzothienyl, benzofuranyl, benzisoxazolyl, imidazo[1,2-b]thiazolyl, purinyl, triazinyl, thieno[3,2-b]pyridinyl, imidazo[1,2-a]pyridinyl, 1,5-naphthyridinyl, 1H-pyrazolo[4,3-b]pyridinyl, oxadiazolyl (e.g., 1,2,3-oxadiazolyl, 1,2,4-oxadiazolyl, 1,3,4-oxadiazolyl), 1,2-dihydro-1,2-azoborinyl, and the like.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, or S, and wherein the ring-forming carbon atoms and heteroatoms of the heterocycloalkyl group can be optionally substituted by one or more oxo or sulfido (e.g., C(O), S(O), C(S), or S(O)₂, etc.). Heterocycloalkyl groups include monocyclic and polycyclic (e.g., having 2 fused rings) systems. Included in heterocycloalkyl are monocyclic and polycyclic 4-10-, 4-7-, and 5-6-membered heterocycloalkyl groups. Heterocycloalkyl groups can also include spirocycles and bridged rings. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl group contains 4 to 10 ring-forming atoms, 4 to 7 ring-forming atoms, 4 to 6 ring-forming atoms or 5 to 6 ring-forming atoms. In some embodiments, the heterocycloalkyl group has 1 to 4 heteroatoms, 1 to 3 heteroatoms, 1 to 2 heteroatoms or 1 heteroatom.

In some embodiments, the heterocycloalkyl is a 4-10 membered monocyclic, bicyclic, or tricyclic heterocycloalkyl having 1, 2, 3, or 4 ring-forming heteroatoms independently selected from N, O, and S, wherein 1, 2, 3, or 4 ring-forming carbon or heteroatoms can be optionally substituted by one or more oxo or sulfido. In some embodiments, the heterocycloalkyl is a 4-10 membered bicyclic heterocycloalkyl having 1, 2, 3, or 4 ring-forming heteroatoms independently selected from N, O, and S, wherein 1, 2, 3, or 4 ring-forming carbon or heteroatoms can be optionally substituted by one or more oxo or sulfido. In some embodiments, the heterocycloalkyl is a 4-7 membered monocyclic heterocycloalkyl having 1 or 2 ring-forming heteroatoms independently selected from N, O, and S, and wherein 1, 2 or 3 ring-forming carbon or heteroatoms can be optionally substituted by one or more oxo or sulfido. In some embodiments, the heterocycloalkyl is a monocyclic 4-6 membered heterocycloalkyl having 1 or 2 heteroatoms independently selected from N, O, S, and B and having one or more oxidized ring members.

Examples of heterocycloalkyl groups include pyrrolidin-2-one, 1,3-isoxazolidin-2-one, pyranyl, tetrahydropyran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, 1,2,3,4-tetrahydroisoquinoline, azabicyclo[3.1.0]hexanyl, diazabicyclo[3.1.0]hexanyl, oxabicyclo[2.1.1]hexanyl, azabicyclo[2.2.1]heptanyl, azabicyclo[2.2.1]heptan-7-yl, azabicyclo[2.2.1]heptan-2-yl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxabicyclo[2.2.2]octanyl, azabicyclo[2.2.2]octanyl, azaadamantanyl, diazaadamantanyl, oxa-adamantanyl, azaspiro[3.3]heptanyl, diazaspiro[3.3]heptanyl, oxa-azaspiro[3.3]heptanyl, azaspiro[3.4]octanyl, diazaspiro[3.4]octanyl, oxa-azaspiro[3.4]octanyl, azaspiro[2.5]octanyl, diazaspiro[2.5]octanyl, azaspiro[4.4]nonanyl, diazaspiro[4.4]nonanyl, oxa-azaspiro[4.4]nonanyl, azaspiro[4.5]decanyl, diazaspiro[4.5]decanyl, diazaspiro[4.4]nonanyl, oxa-diazaspiro[4.4]nonanyl, and the like.

As used herein, “Cₒ₋ₚ cycloalkyl-Cₙ₋ₘ alkyl-” refers to a group of formula cycloalkyl-alkylene-, wherein the cycloalkyl has o to p carbon atoms and the alkylene linking group has n to m carbon atoms.

As used herein “Cₒ₋ₚ aryl-Cₙ₋ₘ alkyl-” refers to a group of formula aryl-alkylene-, wherein the aryl has o to p carbon ring members and the alkylene linking group has n to m carbon atoms.

As used herein, “heteroaryl-Cₙ₋ₘ alkyl-” refers to a group of formula heteroaryl-alkylene-, wherein alkylene linking group has n to m carbon atoms.

As used herein “heterocycloalkyl-Cₙ₋ₘ alkyl-” refers to a group of formula heterocycloalkyl-alkylene-, wherein alkylene linking group has n to m carbon atoms.

As used herein, the term “alkylene” refers a divalent straight chain or branched alkyl linking group. Examples of “alkylene groups” include methylene, ethan-1,1-diyl, ethan-1,2-diyl, propan-1,3-dilyl, propan-1,2-diyl, propan-1,1-diyl and the like.

As used herein, the term “alkenylene” refers a divalent straight chain or branched alkenyl linking group. Examples of “alkenylene groups” include ethen-1,1-diyl, ethen-1,2-diyl, propen-1,3-diyl, 2-buten-1,4-diyl, 3-penten-1,5-diyl, 3-hexen-1,6-diyl, 3-hexen-1,5-diyl, and the like.

As used herein, the term “alkynylene” refers a divalent straight chain or branched alkynyl linking group. Examples of “alkynylene groups” include propyn-1,3-diyl, 2-butyn-1,4-diyl, 3-pentyn-1,5-diyl, 3-hexyn-1,6-diyl, 3-hexyn-1,5-diyl, and the like.

As used herein, an “alkyl linking group” is a bivalent straight chain or branched alkyl linking group (“alkylene group”). For example, “Cₒ₋ₚ cycloalkyl-Cₙ₋ₘ alkyl-”, “Cₒ₋ₚ aryl-Cₙ₋ₘ alkyl-”, “phenyl-Cₙ₋ₘ alkyl-”, “heteroaryl-Cₙ₋ₘ alkyl-”, and “heterocycloalkyl-Cₙ₋ₘ alkyl-” contain alkyl linking groups. Examples of “alkyl linking groups” or “alkylene groups” include methylene, ethan-1,1-diyl, ethan-1,2-diyl, propan-1,3-dilyl, propan-1,2-diyl, propan-1,1-diyl and the like.

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl or sulfonyl group.

As used herein, the term “independently selected from” means that each occurrence of a variable or substituent are independently selected at each occurrence from the applicable list.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula (I), (II), etc.) provided herein include stereoisomers of the compounds.

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution. It is intended that the claims to one tautomer cover both tautomers even if not specified.

All compounds, and pharmaceutically acceptable salts thereof, can be found together with other substances such as water and solvents (e.g., hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis

As will be appreciated by those skilled in the art, the compounds provided herein, including salts and stereoisomers thereof, can be prepared using known organic synthesis techniques and can be synthesized according to any of numerous possible synthetic routes, such as those provided in the Schemes below.

The reactions for preparing compounds described herein can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

The expressions, “ambient temperature” or “room temperature” or “r.t.” as used herein, are understood in the art, and refer generally to a temperature, e.g., a reaction temperature, that is about the temperature of the room in which the reaction is carried out, for example, a temperature from about 20° C. to about 30° C.

Preparation of compounds of the invention can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., March's Advanced Organic Chemistry: Reactions, Mechanisms, and Structure, 6^{th} Ed. (Wiley, 2007); Peturssion et al., “Protecting Groups in Carbohydrate Chemistry,” J. Chem. Educ., 1997, 74(11), 1297; and Wuts et al., Protective Groups in Organic Synthesis, 4th Ed., (Wiley, 2006).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

The Schemes below provide general guidance in connection with preparing the compounds of the invention. One skilled in the art would understand that the preparations shown in the Schemes can be modified or optimized using general knowledge of organic chemistry to prepare various compounds of the invention.

As will be appreciated by those skilled in the art, the compounds provided herein, including salts and stereoisomers thereof, can be prepared using known organic synthesis techniques and can be synthesized according to any of numerous possible synthetic routes.

Compounds of Formula (I) can be synthesized, for example, according to the process shown in Scheme 1. As depicted in Scheme 1, nucleophilic aromatic substitution reactions of compounds of Formula 1-1 (i.e., where Hal is a suitable halogen, such as Br or I) and amines of Formula 1-2 under appropriate conditions (e.g., in the presence of a suitable base, such as NEt(i-Pr)₂, in a suitable solvent, such as CH₃CN) generates compounds of Formula 1-3. Compounds of Formula 1-1 are commercially available, or can be synthesized, for example, by the process depicted in Scheme 2. Transition metal (e.g., Pd, Cu, Ni) catalyzed reactions (including, but not limited to, Suzuki, Stille, Negishi, or Ullman couplings) of compounds of Formula 1-3 and appropriate coupling partners of Formula 1-4 (e.g., heteroaryl boronic acids/esters, heteroaryl trialkyl tin, heteroaryl zinc reagents, or aromatic nitrogen heterocycles) affords compounds of Formula (I). For example, compounds of Formula (I) where ring moiety A is a 4-pyrazolyl group can be accessed via Suzuki reactions between compounds of Formula 1-3 and commercially available 1-(1-ethoxyethyl)-4-(4,4,5,5-tetramethyl-1,3,2-dioxaborolan-2-yl)-1H-pyrazole under appropriate conditions (e.g., in the presence of a palladium catalyst, such as XPhosPd G2, and a base, such as Cs₂CO₃, in a suitable solvent system, such as 1,4-dioxane/H₂O).

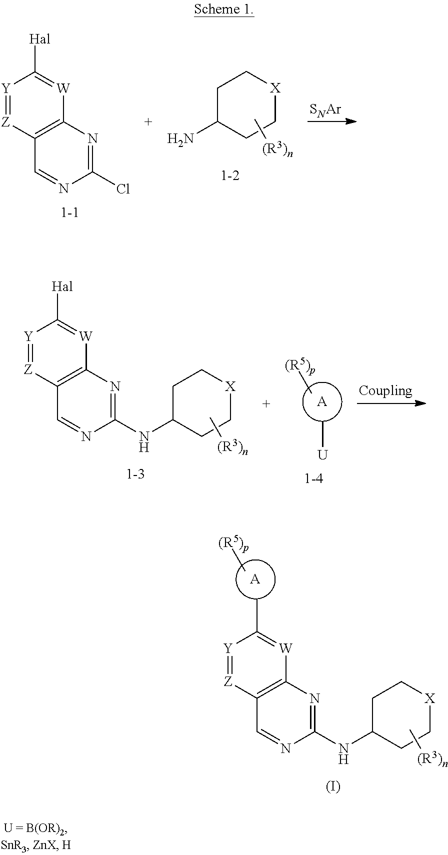

Compounds of Formula 1-1 can be synthesized, for example, according to the process shown in Scheme 2. As depicted in Scheme 2, reduction of compounds of Formula 2-1 using appropriate conditions (e.g., using a suitable reducing agent, such as NaBH₄, in a suitable solvent system, such as THF/MeOH) gives compound 2-2. Oxidation of 2-2 under appropriate conditions (e.g., using a suitable oxidant, such as MnO₂, in a suitable solvent, such as CH₂Cl₂/THF) affords compounds of Formula 1-1.

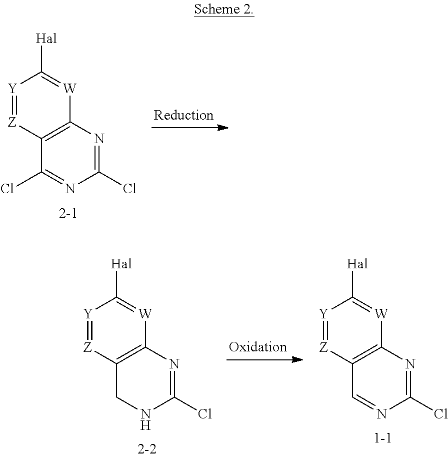

Compounds of Formula (I) can be prepared, for example, using the process shown in Scheme 3. In the process depicted in Scheme 3, borylation of compounds of Formula 1-3 under appropriate conditions (e.g., using a borylating reagent, such as bis(pinacolato)diboron, in the presence of a suitable catalyst, such as Pd(dppf)Cl₂, and a base, such as KOAc, in an appropriate solvent, such as 1,4-dioxane) affords compounds of Formula 3-1. Transition metal (e.g., Pd, Cu, Ni) catalyzed reactions (including, but not limited to, Suzuki or Chan-Lam couplings) of compounds of Formula 3-1 and appropriate coupling partners of Formula 3-2 (i.e., where V is a suitable group, such as a halogen, triflate, or hydrogen) using suitable conditions (e.g., using a suitable catalyst, such as XPhos Pd G2, and a base, such as Cs₂CO₃, in an appropriate solvent, such as 1,4-dioxane) affords compounds of Formula (I).

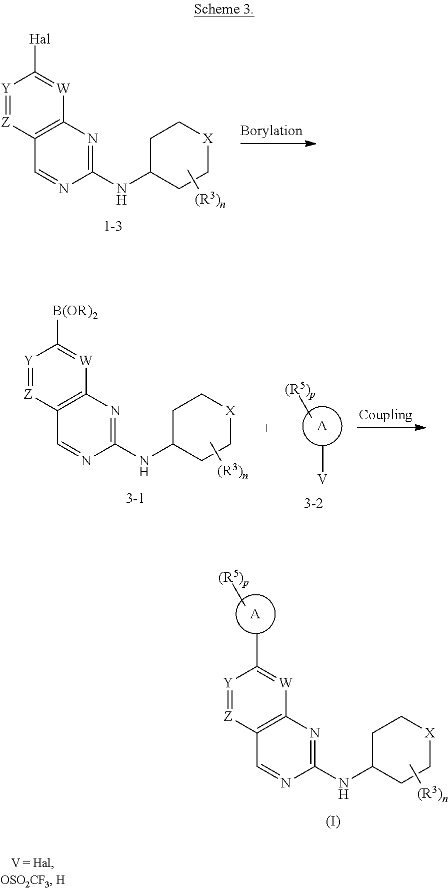

Compounds of Formula (I) can be prepared, for example, using the process shown in Scheme 4. Transition metal (e.g., Pd, Cu, Ni) catalyzed reactions (including, but not limited to, Suzuki, Stille, Negishi, or Ullman couplings) of compounds of Formula 4-1 (i.e., where Hal is an appropriate halogen, such as Cl, Br, or I) and appropriate coupling partners of Formula 1-4 (e.g., heteroaryl boronic acids/esters, heteroaryl trialkyl tin, heteroaryl zinc reagents, or aromatic nitrogen heterocycles) affords compounds of Formula 4-2. Cyclization reactions of compounds of Formula 4-2 with a suitable reagent (e.g., guanidine hydrochloride or guanidine carbonate) under appropriate conditions (e.g., in the presence of a base, such as K₂CO₃, in a suitable solvent, such as dimethylacetamide) furnishes compounds of Formula 4-3. Halogenation of compounds of Formula 4-3 (e.g., via Sandmeyer reaction) under appropriate conditions (in the presence of suitable reagents, such as tetrabutylammonium chloride, trimethylchlorosilane, and tert-butyl nitrite, in an appropriate solvent, such as dimethylformamide) gives compounds of Formula 4-4. A number of methods (e.g., nucleophilic aromatic substitution or a suitable cross-coupling reaction) can be used to access compounds of Formula (I). For example, compounds of Formula 4-4 (i.e., each Hal is a suitable halogen, such as Cl) can be reacted with an appropriate amine nucleophile of Formula 1-2 under appropriate conditions (e.g., in the presence of a suitable reagent, such as CsF, in an appropriate solvent, such as CH₃CN) to generate compounds of Formula (I). Alternatively, transition metal (e.g., Pd, Cu, Ni) catalyzed reactions (including, but not limited to, Buchwald couplings) of compounds 4-4 and appropriate amine nucleophiles of the general Formula 1-2 affords compounds of Formula (I).

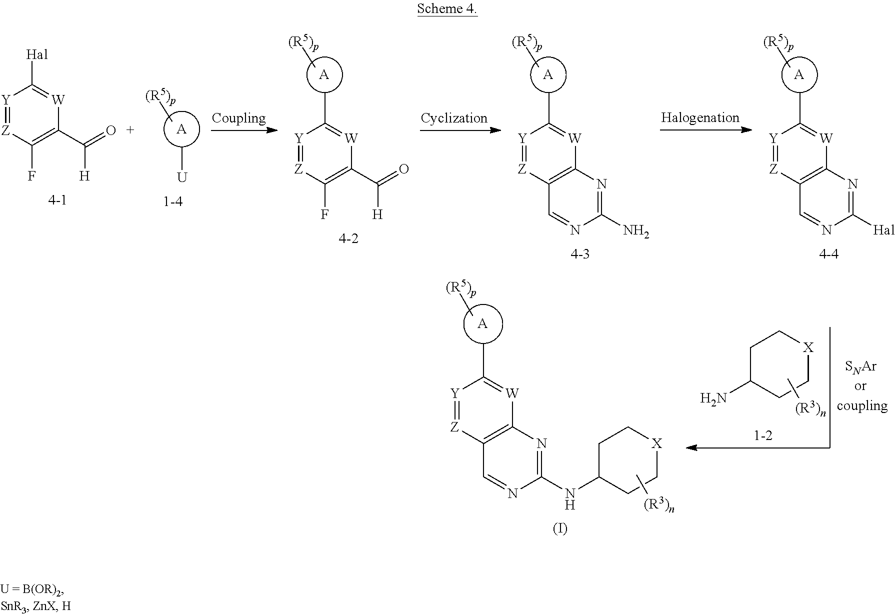

### Methods of Use

Compounds of the present disclosure (and pharmaceutically acceptable salts thereof) can inhibit CDK4 and therefore are useful for treating diseases wherein the underlying pathology is, wholly or partially, mediated by CDK4. In some embodiments, the compounds, and pharmaceutically acceptable salts thereof, inhibit CDK4. In some embodiments, the compounds, and pharmaceutically acceptable salts thereof, are selective for CDK4 over CDK1, CDK6, and/or CDK7. In some embodiments, the compounds, and pharmaceutically acceptable salts thereof, are selective for CDK4 over CDK1. In some embodiments, the compounds, and pharmaceutically acceptable salts thereof, are selective for CDK4 over CDK7. In some embodiments, the compounds, and pharmaceutically acceptable salts thereof, are selective for CDK4 over CDK6. In some embodiments, the compounds, and pharmaceutically acceptable salts thereof, inhibit CDK2 and CDK4. In some embodiments, the compounds, and pharmaceutically acceptable salts thereof, are selective for CDK4 over CDK6. In some embodiments, the compounds, and pharmaceutically acceptable salts thereof, are selective for CDK4 over CDK6. In some embodiments, the selectivity referenced in each of the embodiments of this paragraph is at least 2-fold, at least 3-fold, at least 5-fold, or at least 10-fold.

Such diseases include cancer and other diseases with proliferation disorder. In some embodiments, the present disclosure provides treatment of an individual or a patient in vivo using a compound of Formula (I) or a salt thereof such that growth of cancerous tumors is inhibited. A compound of Formula (I) or of any of the formulas as described herein, or a compound as recited in any of the claims and described herein, or a salt thereof, can be used to inhibit the growth of cancerous tumors with aberrations that activate the CDK4 kinase activity. These include, but are not limited to, disease (e.g., cancers) that are characterized by amplification or overexpression of CCNE1 such as ovarian cancer, uterine carcinosarcoma and breast cancer and p27 inactivation such as breast cancer and melanomas. Accordingly, in some embodiments of the methods, the patient has been previously determined to have an amplification of the cyclin E1 (CCNE1) gene and/or an expression level of CCNE1 in a biological sample obtained from the human subject that is higher than a control expression level of CCNE1. Alternatively, a compound of Formula (I) or of any of the formulas as described herein, or a compound as recited in any of the claims and described herein, or a salt thereof, can be used in conjunction with other agents or standard cancer treatments, as described below. In one embodiment, the present disclosure provides a method for inhibiting growth of tumor cells in vitro. The method includes contacting the tumor cells in vitro with a compound of Formula (I) or of any of the formulas as described herein, or of a compound as recited in any of the claims and described herein, or of a salt thereof. In another embodiment, the present disclosure provides a method for inhibiting growth of tumor cells with CCNE1 amplification and overexpression in an individual or a patient. The method includes administering to the individual or patient in need thereof a therapeutically effective amount of a compound of Formula (I) or of any of the formulas as described herein, or of a compound as recited in any of the claims and described herein, or a salt or a stereoisomer thereof.

In some embodiments, provided herein is a method of inhibiting CDK4, comprising contacting the CDK4 with a compound of Formula (I) or any of the formulas as described herein, a compound as recited in any of the claims and described herein, or a salt thereof. In some embodiments, provided herein is a method of inhibiting CDK4 in a patient, comprising administering to the patient a compound of Formula (I) or any of the formulas as described herein, a compound as recited in any of the claims and described herein, or a salt thereof.

In some embodiments, provided herein is a method for treating cancer. The method includes administering to a patient (in need thereof), a therapeutically effective amount of a compound of Formula (I) or any of the formulas as described herein, a compound as recited in any of the claims and described herein, or a salt thereof. In another embodiment, the cancer is characterized by amplification or overexpression of CCNE1. In some embodiments, the cancer is ovarian cancer or breast cancer, characterized by amplification or overexpression of CCNE1.

In some embodiments, provided herein is a method of treating a disease or disorder associated with CDK4 in a patient, comprising administering to the patient a therapeutically effective amount of a compound of Formula (I) or any of the formulas as described herein, a compound as recited in any of the claims and described herein, or a salt thereof. In some embodiments, the disease or disorder associated with CDK4 is associated with an amplification of the cyclin E1 (CCNE1) gene and/or overexpression of CCNE1.

In some embodiments, the disease or disorder associated with CDK4 is N-myc amplified neuroblastoma cells (see Molenaar, et al., Proc Natl Acad Sci USA 106(31): 12968-12973) K-Ras mutant lung cancers (see Hu, S., et al., Mol Cancer Ther, 2015. 14(11): 2576-85, and cancers with FBW7 mutation and CCNE1 overexpression (see Takada et al., Cancer Res, 2017. 77(18): 4881-4893).

In some embodiments, the disease or disorder associated with CDK4 is lung squamous cell carcinoma, lung adenocarcinoma, pancreatic adenocarcinoma, breast invasive carcinoma, uterine carcinosarcoma, ovarian serous cystadenocarcinoma, stomach adenocarcinoma, esophageal carcinoma, bladder urothelial carcinoma, mesothelioma, or sarcoma.

In some embodiments, the disease or disorder associated with CDK4 is lung adenocarcinoma, breast invasive carcinoma, uterine carcinosarcoma, ovarian serous cystadenocarcinoma, or stomach adenocarcinoma.

In some embodiments, the disease or disorder associated with CDK4 is an adenocarcinoma, carcinoma, or cystadenocarcinoma.

In some embodiments, the disease or disorder associated with CDK4 is uterine cancer, ovarian cancer, stomach cancer, esophageal cancer, lung cancer, bladder cancer, pancreatic cancer, or breast cancer.

In some embodiments, the disease or disorder associated with CDK4 is a cancer.

In some embodiments, the cancer is characterized by amplification or overexpression of CCNE1. In some embodiments, the cancer is ovarian cancer or breast cancer, characterized by amplification or overexpression of CCNE1.

In some embodiments, the breast cancer is chemotherapy or radiotherapy resistant breast cancer, endocrine resistant breast cancer, trastuzumab resistant breast cancer, or breast cancer demonstrating primary or acquired resistance to CDK4/6 inhibition. In some embodiments, the breast cancer is advanced or metastatic breast cancer.

Examples of cancers that are treatable using the compounds of the present disclosure include, but are not limited to, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The compounds of the present disclosure are also useful for the treatment of metastatic cancers.

In some embodiments, cancers treatable with compounds of the present disclosure include melanoma (e.g., metastatic malignant melanoma, BRAF and HSP90 inhibition-resistant melanoma), renal cancer (e.g., clear cell carcinoma), prostate cancer (e.g., hormone refractory prostate adenocarcinoma), breast cancer, colon cancer, lung cancer (e.g., non-small cell lung cancer and small cell lung cancer), squamous cell head and neck cancer, urothelial cancer (e.g., bladder) and cancers with high microsatellite instability (MSI^{high}). Additionally, the disclosure includes refractory or recurrent malignancies whose growth may be inhibited using the compounds of the disclosure.

In some embodiments, cancers that are treatable using the compounds of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including follicular lymphoma, including relapsed or refractory NHL and recurrent follicular), Hodgkin lymphoma or multiple myeloma) and combinations of said cancers.

In some embodiments, cancers that are treatable using the compounds of the present disclosure include, but are not limited to, cholangiocarcinoma, bile duct cancer, triple negative breast cancer, rhabdomyosarcoma, small cell lung cancer, leiomyosarcoma, hepatocellular carcinoma, Ewing's sarcoma, brain cancer, brain tumor, astrocytoma, neuroblastoma, neurofibroma, basal cell carcinoma, chondrosarcoma, epithelioid sarcoma, eye cancer, Fallopian tube cancer, gastrointestinal cancer, gastrointestinal stromal tumors, hairy cell leukemia, intestinal cancer, islet cell cancer, oral cancer, mouth cancer, throat cancer, laryngeal cancer, lip cancer, mesothelioma, neck cancer, nasal cavity cancer, ocular cancer, ocular melanoma, pelvic cancer, rectal cancer, renal cell carcinoma, salivary gland cancer, sinus cancer, spinal cancer, tongue cancer, tubular carcinoma, urethral cancer, and ureteral cancer.

In some embodiments, the compounds of the present disclosure can be used to treat sickle cell disease and sickle cell anemia.

In some embodiments, diseases and indications that are treatable using the compounds of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CMIL), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), and essential thrombocytosis (ET), myelodysplasia syndrome (MDS), T-cell acute lymphoblastic lymphoma (T-ALL) and multiple myeloma (MM).

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, and teratoma.

Exemplary lung cancers include non-small cell lung cancer (NSCLC), small cell lung cancer (SCLC), bronchogenic carcinoma, squamous cell, undifferentiated small cell, undifferentiated large cell, adenocarcinoma, alveolar (bronchiolar) carcinoma, bronchial adenoma, chondromatous hamartoma, and mesothelioma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer.

Exemplary genitourinary tract cancers include cancers of the kidney (adenocarcinoma, Wilm's tumor [nephroblastoma]), bladder and urethra (squamous cell carcinoma, transitional cell carcinoma, adenocarcinoma), prostate (adenocarcinoma, sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma).

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma, and giant cell tumors

Exemplary nervous system cancers include cancers of the skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma, glioblastoma multiform, oligodendroglioma, schwannoma, retinoblastoma, congenital tumors), and spinal cord (neurofibroma, meningioma, glioma, sarcoma), as well as neuroblastoma and Lhermitte-Duclos disease.

Exemplary gynecological cancers include cancers of the uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, Merkel cell carcinoma, squamous cell carcinoma, Kaposi's sarcoma, moles dysplastic nevi, lipoma, angioma, dermatofibroma, and keloids. In some embodiments, diseases and indications that are treatable using the compounds of the present disclosure include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

It is believed that compounds of Formula (I), or any of the embodiments thereof, may possess satisfactory pharmacological profile and promising biopharmaceutical properties, such as toxicological profile, metabolism and pharmacokinetic properties, solubility, and permeability. It will be understood that determination of appropriate biopharmaceutical properties is within the knowledge of a person skilled in the art, e.g., determination of cytotoxicity in cells or inhibition of certain targets or channels to determine potential toxicity.

The terms “individual”, “patient,” and “subject” used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

### Combination Therapies

### I. Cancer Therapies

Cancer cell growth and survival can be impacted by dysfunction in multiple signaling pathways. Thus, it is useful to combine different enzyme/protein/receptor inhibitors, exhibiting different preferences in the targets which they modulate the activities of, to treat such conditions. Targeting more than one signaling pathway (or more than one biological molecule involved in a given signaling pathway) may reduce the likelihood of drug-resistance arising in a cell population, and/or reduce the toxicity of treatment.

One or more additional pharmaceutical agents such as, for example, chemotherapeutics, anti-inflammatory agents, steroids, immunosuppressants, immune-oncology agents, metabolic enzyme inhibitors, chemokine receptor inhibitors, and phosphatase inhibitors, as well as targeted therapies such as Bcr-Abl, Flt-3, EGFR, HER2, JAK, c-MET, VEGFR, PDGFR, c-Kit, IGF-1R, RAF, FAK, and CDK4/6 kinase inhibitors such as, for example, those described in WO 2006/056399 can be used in combination with the compounds of the present disclosure for treatment of CDK4-associated diseases, disorders or conditions. Other agents such as therapeutic antibodies can be used in combination with the compounds of the present disclosure for treatment of CDK4-associated diseases, disorders or conditions. The one or more additional pharmaceutical agents can be administered to a patient simultaneously or sequentially.

In some embodiments, the compound or salt is administered or used in combination with a BCL2 inhibitor.

The compounds as disclosed herein can be used in combination with one or more other enzyme/protein/receptor inhibitors therapies for the treatment of diseases, such as cancer and other diseases or disorders described herein. Examples of diseases and indications treatable with combination therapies include those as described herein. Examples of cancers include solid tumors and non-solid tumors, such as liquid tumors, and blood cancers. Examples of infections include viral infections, bacterial infections, fungus infections or parasite infections. For example, the compounds of the present disclosure can be combined with one or more inhibitors of the following kinases for the treatment of cancer: Akt1, Akt2, Akt3, BCL2, CDK4/6, TGF-βR, PKA, PKG, PKC, CaM-kinase, phosphorylase kinase, MEKK, ERK, MAPK, mTOR, EGFR, HER2, HER3, HER4, INS-R, IDH2, IGF-1R, IR-R, PDGFαR, PDGFβR, PI3K (alpha, beta, gamma, delta, and multiple or selective), CSF1R, KIT, FLK-II, KDR/FLK-1, FLK-4, fit-1, FGFR1, FGFR2, FGFR3, FGFR4, c-Met, PARP, Ron, Sea, TRKA, TRKB, TRKC, TAM kinases (Axl, Mer, Tyro3), FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. In some embodiments, the compounds of the present disclosure can be combined with one or more of the following inhibitors for the treatment of cancer or infections. Non-limiting examples of inhibitors that can be combined with the compounds of the present disclosure for treatment of cancer and infections include an FGFR inhibitor (FGFR1, FGFR2, FGFR3 or FGFR4, e.g., pemigatinib (INCB54828), INCB62079), an EGFR inhibitor (also known as ErB-1 or HER-1; e.g., erlotinib, gefitinib, vandetanib, orsimertinib, cetuximab, necitumumab, or panitumumab), a VEGFR inhibitor or pathway blocker (e.g. bevacizumab, pazopanib, sunitinib, sorafenib, axitinib, regorafenib, ponatinib, cabozantinib, vandetanib, ramucirumab, lenvatinib, ziv-aflibercept), a PARP inhibitor (e.g., olaparib, rucaparib, veliparib or niraparib), a JAK inhibitor (JAK1 and/or JAK2, e.g., ruxolitinib or baricitinib; JAK1, e.g., itacitinib (INCB39110), INCB052793, or INCB054707), an IDO inhibitor (e.g., epacadostat, NLG919, or BMS-986205, MK7162), an LSD1 inhibitor (e.g., GSK2979552, INCB59872 and INCB60003), a TDO inhibitor, a PI3K-delta inhibitor (e.g., parsaclisib (INCB50465) or INCB50797), a PI3K-gamma inhibitor such as PI3K-gamma selective inhibitor, a Pim inhibitor (e.g., INCB53914), a CSF1R inhibitor, a TAM receptor tyrosine kinases (Tyro-3, Axl, and Mer; e.g., INCB081776), an adenosine receptor antagonist (e.g., A2a/A2b receptor antagonist), an HPK1 inhibitor, a chemokine receptor inhibitor (e.g., CCR2 or CCR5 inhibitor), a SHP1/2 phosphatase inhibitor, a histone deacetylase inhibitor (HDAC) such as an HDAC₈ inhibitor, an angiogenesis inhibitor, an interleukin receptor inhibitor, bromo and extra terminal family members inhibitors (for example, bromodomain inhibitors or BET inhibitors such as INCB54329 and INCB57643), c-MET inhibitors (e.g., capmatinib), an anti-CD19 antibody (e.g., tafasitamab), an ALK2 inhibitor (e.g., INCB00928); or combinations thereof.

In some embodiments, the compound or salt described herein is administered with a PI3Kδ inhibitor. In some embodiments, the compound or salt described herein is administered with a JAK inhibitor. In some embodiments, the compound or salt described herein is administered with a JAK1 or JAK2 inhibitor (e.g., baricitinib or ruxolitinib). In some embodiments, the compound or salt described herein is administered with a JAK1 inhibitor. In some embodiments, the compound or salt described herein is administered with a JAK1 inhibitor, which is selective over JAK2.

Example antibodies for use in combination therapy include, but are not limited to, trastuzumab (e.g., anti-HER2), ranibizumab (e.g., anti-VEGF-A), bevacizumab (AVASTIN™ e.g., anti-VEGF), panitumumab (e.g., anti-EGFR), cetuximab (e.g., anti-EGFR), rituxan (e.g., anti-CD20), and antibodies directed to c-MET.

One or more of the following agents may be used in combination with the compounds of the present disclosure and are presented as a non-limiting list: a cytostatic agent, cisplatin, doxorubicin, taxotere, taxol, etoposide, irinotecan, camptosar, topotecan, paclitaxel, docetaxel, epothilones, tamoxifen, 5-fluorouracil, methotrexate, temozolomide, cyclophosphamide, SCH 66336, R115777, L778,123, BMS 214662, IRESSA™ (gefitinib), TARCEVA™ (erlotinib), antibodies to EGFR, intron, ara-C, adriamycin, cytoxan, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, oxaliplatin, leucovirin, ELOXATIN™ (oxaliplatin), pentostatine, vinblastine, vincristine, vindesine, bleomycin, dactinomycin, daunorubicin, doxorubicin, epirubicin, idarubicin, mithramycin, deoxycoformycin, mitomycin-C, L-asparaginase, teniposide, 17-alpha-ethinylestradiol, diethylstilbestrol, testosterone, prednisone, fluoxymesterone, dromostanolone propionate, testolactone, megestrolacetate, methylprednisolone, methyltestosterone, prednisolone, triamcinolone, chlorotrianisene, hydroxyprogesterone, aminoglutethimide, estramustine, medroxyprogesterone acetate, leuprolide, flutamide, toremifene, goserelin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, avastin, HERCEPTIN™ (trastuzumab), BEXXAR™ (tositumomab), VELCADE™ (bortezomib), ZEVALIN™ (ibritumomab tiuxetan), TRISENOX™ (arsenic trioxide), XELODA™ (capecitabine), vinorelbine, porfimer, ERBITUX™ (cetuximab), thiotepa, altretamine, melphalan, trastuzumab, lerozole, fulvestrant, exemestane, ifosfomide, rituximab, C225 (cetuximab), Campath (alemtuzumab), clofarabine, cladribine, aphidicolon, rituxan, sunitinib, dasatinib, tezacitabine, Sml1, fludarabine, pentostatin, triapine, didox, trimidox, amidox, 3-AP, and MDL-101,731.

The compounds of the present disclosure can further be used in combination with other methods of treating cancers, for example by chemotherapy, irradiation therapy, tumor-targeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, bispecific or multi-specific antibody, antibody drug conjugate, adoptive T cell transfer, Toll receptor agonists, RIG-I agonists, oncolytic virotherapy and immunomodulating small molecules, including thalidomide or JAK1/2 inhibitor, PI3Kδ inhibitor and the like. The compounds can be administered in combination with one or more anti-cancer drugs, such as a chemotherapeutic agent. Examples of chemotherapeutics include any of: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, and zoledronate.

Additional examples of chemotherapeutics include proteasome inhibitors (e.g., bortezomib), thalidomide, revlimid, and DNA-damaging agents such as melphalan, doxorubicin, cyclophosphamide, vincristine, etoposide, carmustine, and the like.

Example steroids include corticosteroids such as dexamethasone or prednisone.

Example Bcr-Abl inhibitors include imatinib mesylate (GLEEVAC™), nilotinib, dasatinib, bosutinib, and ponatinib, and pharmaceutically acceptable salts. Other example suitable Bcr-Abl inhibitors include the compounds, and pharmaceutically acceptable salts thereof, of the genera and species disclosed in U.S. Pat. No. 5,521,184, WO 04/005281, and U.S. Ser. No. 60/578,491.

Example suitable Flt-3 inhibitors include midostaurin, lestaurtinib, linifanib, sunitinib, sunitinib, maleate, sorafenib, quizartinib, crenolanib, pacritinib, tandutinib, PLX3397 and ASP2215, and their pharmaceutically acceptable salts. Other example suitable Flt-3 inhibitors include compounds, and their pharmaceutically acceptable salts, as disclosed in WO 03/037347, WO 03/099771, and WO 04/046120.

Example suitable RAF inhibitors include dabrafenib, sorafenib, and vemurafenib, and their pharmaceutically acceptable salts. Other example suitable RAF inhibitors include compounds, and their pharmaceutically acceptable salts, as disclosed in WO 00/09495 and WO 05/028444.

Example suitable FAK inhibitors include VS-4718, VS-5095, VS-6062, VS-6063, BI853520, and GSK2256098, and their pharmaceutically acceptable salts. Other example suitable FAK inhibitors include compounds, and their pharmaceutically acceptable salts, as disclosed in WO 04/080980, WO 04/056786, WO 03/024967, WO 01/064655, WO 00/053595, and WO 01/014402.

Example suitable CDK4/6 inhibitors include palbociclib, ribociclib, trilaciclib, lerociclib, and abemaciclib, and their pharmaceutically acceptable salts. Other example suitable CDK4/6 inhibitors include compounds, and their pharmaceutically acceptable salts, as disclosed in WO 09/085185, WO 12/129344, WO 11/101409, WO 03/062236, WO 10/075074, and WO 12/061156.

In some embodiments, the compounds or salt of the present disclosure can be combined with a CDK2 inhibitor. In some embodiments, the CDK2 inhibitor is any of the compounds or salts disclosed in U.S. Pat. No. 11,427,567, which is herein incorporated by reference in its entirety. In some embodiments, the CDK2 inhibitor is any of the compounds or salts disclosed in U.S. Pat. No. 11,851,426, which is herein incorporated by reference in its entirety.

In some embodiments, the compounds of the disclosure can be used in combination with one or more other kinase inhibitors including imatinib, particularly for treating patients resistant to imatinib or other kinase inhibitors.

In some embodiments, the compounds of the disclosure can be used in combination with a chemotherapeutic in the treatment of cancer, and may improve the treatment response as compared to the response to the chemotherapeutic agent alone, without exacerbation of its toxic effects. In some embodiments, the compounds of the disclosure can be used in combination with a chemotherapeutic provided herein. For example, additional pharmaceutical agents used in the treatment of multiple myeloma, can include, without limitation, melphalan, melphalan plus prednisone [MP], doxorubicin, dexamethasone, and Velcade (bortezomib). Further additional agents used in the treatment of multiple myeloma include Bcr-Abl, Flt-3, RAF and FAK kinase inhibitors. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM). Additive or synergistic effects are desirable outcomes of combining a compound or salt of the present disclosure with an additional agent.

The agents can be combined with the present compound in a single or continuous dosage form, or the agents can be administered simultaneously or sequentially as separate dosage forms.

The compounds of the present disclosure can be used in combination with one or more other inhibitors or one or more therapies for the treatment of infections. Examples of infections include viral infections, bacterial infections, fungus infections or parasite infections.

In some embodiments, a corticosteroid such as dexamethasone is administered to a patient in combination with the compounds of the disclosure where the dexamethasone is administered intermittently as opposed to continuously.

The compounds of Formula (I) or any of the formulas as described herein, a compound as recited in any of the claims and described herein, or salts thereof can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds of Formula (I) or any of the formulas as described herein, a compound as recited in any of the claims and described herein, or salts thereof can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). In some embodiments, the compounds of the present disclosure can be used in combination with tumor specific antigen such as heat shock proteins isolated from tumor tissue itself. In some embodiments, the compounds of Formula (I) or any of the formulas as described herein, a compound as recited in any of the claims and described herein, or salts thereof can be combined with dendritic cells immunization to activate potent anti-tumor responses.

The compounds of the present disclosure can be used in combination with bispecific macrocyclic peptides that target Fe alpha or Fe gamma receptor-expressing effectors cells to tumor cells. The compounds of the present disclosure can also be combined with macrocyclic peptides that activate host immune responsiveness.

In some further embodiments, combinations of the compounds of the disclosure with other therapeutic agents can be administered to a patient prior to, during, and/or after a bone marrow transplant or stem cell transplant. The compounds of the present disclosure can be used in combination with bone marrow transplant for the treatment of a variety of tumors of hematopoietic origin.

The compounds of Formula (I) or any of the formulas as described herein, a compound as recited in any of the claims and described herein, or salts thereof can be used in combination with vaccines, to stimulate the immune response to pathogens, toxins, and self-antigens. Examples of pathogens for which this therapeutic approach may be particularly useful include pathogens for which there is currently no effective vaccine, or pathogens for which conventional vaccines are less than completely effective. These include, but are not limited to, HIV, Hepatitis (A, B, & C), Influenza, Herpes, Giardia, Malaria, Leishmania, Staphylococcus aureus, Pseudomonas Aeruginosa.

Viruses causing infections treatable by methods of the present disclosure include, but are not limited to human papillomavirus, influenza, hepatitis A, B, C or D viruses, adenovirus, poxvirus, herpes simplex viruses, human cytomegalovirus, severe acute respiratory syndrome virus, Ebola virus, measles virus, herpes virus (e.g., VZV, HSV-1, HAV-6, HSV-II, and CMV, Epstein Barr virus), flaviviruses, echovirus, rhinovirus, coxsackie virus, cornovirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Pathogenic bacteria causing infections treatable by methods of the disclosure include, but are not limited to, chlamydia, rickettsial bacteria, mycobacteria, staphylococci, streptococci, pneumococci, meningococci and conococci, klebsiella, proteus, serratia, pseudomonas, legionella, diphtheria, salmonella, bacilli, cholera, tetanus, botulism, anthrax, plague, leptospirosis, and Lyme's disease bacteria.

Pathogenic fungi causing infections treatable by methods of the disclosure include, but are not limited to, Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

Pathogenic parasites causing infections treatable by methods of the disclosure include, but are not limited to, Entamoeba histolytica, Balantidium coli, Naegleria fowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondii, and Nippostrongylus brasiliensis.

When more than one pharmaceutical agent is administered to a patient, they can be administered simultaneously, separately, sequentially, or in combination (e.g., for more than two agents).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

### II. Immune-Checkpoint Therapies

Compounds of the present disclosure can be used in combination with one or more immune checkpoint inhibitors for the treatment of diseases, such as cancer or infections. Exemplary immune checkpoint inhibitors include inhibitors against immune checkpoint molecules such as CBL-B, CD20, CD28, CD40, CD70, CD122, CD96, CD73, CD47, CDK2, CDK4, GITR, CSF1R, JAK, PI3K delta, PI3K gamma, TAM, arginase, HPK1, CD137 (also known as 4-1BB), ICOS, A2AR, B7-H3, B7-H4, BTLA, CTLA-4, LAG3, TIM3, TLR (TLR7/8), TIGIT, CD112R, VISTA, PD-1, PD-L1 and PD-L2. In some embodiments, the immune checkpoint molecule is a stimulatory checkpoint molecule selected from CD27, CD28, CD40, ICOS, OX40, GITR and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, TIGIT, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the compounds provided herein can be used in combination with one or more agonists of immune checkpoint molecules, e.g., OX40, CD27, GITR, and CD137 (also known as 4-1BB).

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1 or PD-L1, e.g., an anti-PD-1 or anti-PD-L1 monoclonal antibody. In some embodiments, the anti-PD-1 or anti-PD-L1 antibody is nivolumab, pembrolizumab, atezolizumab, durvalumab, avelumab, cemiplimab, atezolizumab, avelumab, tislelizumab, spartalizumab (PDR001), cetrelimab (JNJ-63723283), toripalimab (JS001), camrelizumab (SHR-1210), sintilimab (IBI308), AB122 (GLS-010), AMP-224, AMP-514/MEDI-0680, BMS936559, JTX-4014, BGB-108, SHR-1210, MEDI4736, FAZ053, BCD-100, KN035, CS1001, BAT1306, LZM009, AK105, HLX10, SHR-1316, CBT-502 (TQB2450), A167 (KL-A167), STI-A101 (ZKAB001), CK-301, BGB-A333, MSB-2311, HLX20, TSR-042, or LY3300054. In some embodiments, the inhibitor of PD-1 or PD-L1 is one disclosed in U.S. Pat. Nos. 7,488,802, 7,943,743, 8,008,449, 8,168,757, 8,217, 149, WO 03042402, WO 2008156712, WO 2010089411, WO 2010036959, WO 2011066342, WO 2011159877, WO 2011082400, or WO 2011161699, which are each incorporated herein by reference in its entirety.

In some embodiments, the antibody is an anti-PD-1 antibody, e.g., an anti-PD-1 monoclonal antibody. In some embodiments, the anti-PD-1 antibody is nivolumab, pembrolizumab, cemiplimab, spartalizumab, camrelizumab, cetrelimab, toripalimab, sintilimab, AB122, AMP-224, JTX-4014, BGB-108, BCD-100, BAT1306, LZM009, AK105, HLX10, or TSR-042. In some embodiments, the anti-PD-1 antibody is nivolumab, pembrolizumab, cemiplimab, spartalizumab, camrelizumab, cetrelimab, toripalimab, or sintilimab. In some embodiments, the anti-PD-1 antibody is pembrolizumab. In some embodiments, the anti-PD-1 antibody is nivolumab. In some embodiments, the anti-PD-1 antibody is cemiplimab. In some embodiments, the anti-PD-1 antibody is spartalizumab. In some embodiments, the anti-PD-1 antibody is camrelizumab. In some embodiments, the anti-PD-1 antibody is cetrelimab. In some embodiments, the anti-PD-1 antibody is toripalimab. In some embodiments, the anti-PD-1 antibody is sintilimab. In some embodiments, the anti-PD-1 antibody is AB122. In some embodiments, the anti-PD-1 antibody is AMP-224. In some embodiments, the anti-PD-1 antibody is JTX-4014. In some embodiments, the anti-PD-1 antibody is BGB-108. In some embodiments, the anti-PD-1 antibody is BCD-100. In some embodiments, the anti-PD-1 antibody is BAT1306. In some embodiments, the anti-PD-1 antibody is LZM009. In some embodiments, the anti-PD-1 antibody is AK105. In some embodiments, the anti-PD-1 antibody is HLX10. In some embodiments, the anti-PD-1 antibody is TSR-042. In some embodiments, the anti-PD-1 monoclonal antibody is nivolumab or pembrolizumab. In some embodiments, the anti-PD-1 monoclonal antibody is MGA012. In some embodiments, the anti-PD1 antibody is SHR-1210. Other anti-cancer agent(s) include antibody therapeutics such as 4-1BB (e.g., urelumab, utomilumab). In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, avelumab, durvalumab, tislelizumab, BMS-935559, MEDI4736, atezolizumab (MPDL3280A; also known as RG7446), avelumab (MSB0010718C), FAZ053, KN035, CS1001, SHR-1316, CBT-502, A167, STI-A101, CK-301, BGB-A333, MSB-2311, HLX20, or LY3300054. In some embodiments, the anti-PD-L1 antibody is atezolizumab, avelumab, durvalumab, or tislelizumab. In some embodiments, the anti-PD-L1 antibody is atezolizumab. In some embodiments, the anti-PD-L1 antibody is avelumab. In some embodiments, the anti-PD-L1 antibody is durvalumab. In some embodiments, the anti-PD-L1 antibody is tislelizumab. In some embodiments, the anti-PD-L1 antibody is BMS-935559. In some embodiments, the anti-PD-L1 antibody is MEDI4736. In some embodiments, the anti-PD-L1 antibody is FAZ053. In some embodiments, the anti-PD-L1 antibody is KN035. In some embodiments, the anti-PD-L1 antibody is CS1001. In some embodiments, the anti-PD-L1 antibody is SHR-1316. In some embodiments, the anti-PD-L1 antibody is CBT-502. In some embodiments, the anti-PD-L1 antibody is A167. In some embodiments, the anti-PD-L1 antibody is STI-A101. In some embodiments, the anti-PD-L1 antibody is CK-301. In some embodiments, the anti-PD-L1 antibody is BGB-A333. In some embodiments, the anti-PD-L1 antibody is MSB-2311. In some embodiments, the anti-PD-L1 antibody is HLX20. In some embodiments, the anti-PD-L1 antibody is LY3300054.

In some embodiments, the inhibitor of an immune checkpoint molecule is a small molecule that binds to PD-L1, or a pharmaceutically acceptable salt thereof. In some embodiments, the inhibitor of an immune checkpoint molecule is a small molecule that binds to and internalizes PD-L1, or a pharmaceutically acceptable salt thereof. In some embodiments, the inhibitor of an immune checkpoint molecule is a compound selected from those in US 2018/0179201, US 2018/0179197, US 2018/0179179, US 2018/0179202, US 2018/0177784, US 2018/0177870, U.S. Ser. No. 16/369,654 (filed Mar. 29, 2019), and U.S. Ser. No. 62/688,164, or a pharmaceutically acceptable salt thereof, each of which is incorporated herein by reference in its entirety.

In some embodiments, the inhibitor of an immune checkpoint molecule is an inhibitor of KIR, TIGIT, LAIR1, CD160, 2B4 and TGFR beta.

In some embodiments, the inhibitor is MCLA-145.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, INCAGN2385, or eftilagimod alpha (IMP321).

In some embodiments, the inhibitor of an immune checkpoint molecule is an inhibitor of CD73. In some embodiments, the inhibitor of CD73 is oleclumab.

In some embodiments, the inhibitor of an immune checkpoint molecule is an inhibitor of TIGIT. In some embodiments, the inhibitor of TIGIT is OMP-31M32.

In some embodiments, the inhibitor of an immune checkpoint molecule is an inhibitor of VISTA. In some embodiments, the inhibitor of VISTA is JNJ-61610588 or CA-170.

In some embodiments, the inhibitor of an immune checkpoint molecule is an inhibitor of B7-H3. In some embodiments, the inhibitor of B7-H3 is enoblituzumab, MGD009, or 8119.

In some embodiments, the inhibitor of an immune checkpoint molecule is an inhibitor of KIR. In some embodiments, the inhibitor of KIR is lirilumab or IPH4102.

In some embodiments, the inhibitor of an immune checkpoint molecule is an inhibitor of A2aR. In some embodiments, the inhibitor of A2aR is CPI-444.

In some embodiments, the inhibitor of an immune checkpoint molecule is an inhibitor of TGF-beta. In some embodiments, the inhibitor of TGF-beta is trabedersen, galusertinib, or M7824.

In some embodiments, the inhibitor of an immune checkpoint molecule is an inhibitor of PI3K-gamma. In some embodiments, the inhibitor of PI3K-gamma is IPI-549.

In some embodiments, the inhibitor of an immune checkpoint molecule is an inhibitor of CD47. In some embodiments, the inhibitor of CD47 is Hu5F9-G4 or TTI-621.

In some embodiments, the inhibitor of an immune checkpoint molecule is an inhibitor of CD73. In some embodiments, the inhibitor of CD73 is MEDI9447.

In some embodiments, the inhibitor of an immune checkpoint molecule is an inhibitor of CD70. In some embodiments, the inhibitor of CD70 is cusatuzumab or BMS-936561.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of CD20, e.g., an anti-CD20 antibody. In some embodiments, the anti-CD20 antibody is obinutuzumab or rituximab.

In some embodiments, the agonist of an immune checkpoint molecule is an agonist of OX40, CD27, CD28, GITR, ICOS, CD40, TLR7/8, and CD137 (also known as 4-1BB).

In some embodiments, the agonist of CD137 is urelumab. In some embodiments, the agonist of CD137 is utomilumab.

In some embodiments, the agonist of an immune checkpoint molecule is an inhibitor of GITR. In some embodiments, the agonist of GITR is TRX518, MK-4166, INCAGN1876, MK-1248, AMG228, BMS-986156, GWN323, MEDI1873, or MEDI6469. In some embodiments, the agonist of an immune checkpoint molecule is an agonist of OX40, e.g., OX40 agonist antibody or OX40L fusion protein. In some embodiments, the anti-OX40 antibody is INCAGN01949, MEDI0562 (tavolimab), MOXR-0916, PF-04518600, GSK3174998, BMS-986178, or 9B12. In some embodiments, the OX40L fusion protein is MEDI6383.

In some embodiments, the agonist of an immune checkpoint molecule is an agonist of CD40. In some embodiments, the agonist of CD40 is CP-870893, ADC-1013, CDX-1140, SEA-CD40, R07009789, JNJ-64457107, APX-005M, or Chi Lob 7/4.

In some embodiments, the agonist of an immune checkpoint molecule is an agonist of ICOS. In some embodiments, the agonist of ICOS is GSK-3359609, JTX-2011, or MEDI-570.

In some embodiments, the agonist of an immune checkpoint molecule is an agonist of CD28. In some embodiments, the agonist of CD28 is theralizumab.

In some embodiments, the agonist of an immune checkpoint molecule is an agonist of CD27. In some embodiments, the agonist of CD27 is varlilumab.

In some embodiments, the agonist of an immune checkpoint molecule is an agonist of TLR7/8. In some embodiments, the agonist of TLR7/8 is MEDI9197.

The compounds of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGFβ receptor. In some embodiments, the bispecific antibody binds to PD-1 and PD-L1. In some embodiments, the bispecific antibody that binds to PD-1 and PD-L1 is MCLA-136. In some embodiments, the bispecific antibody binds to PD-L1 and CTLA-4. In some embodiments, the bispecific antibody that binds to PD-L1 and CTLA-4 is AK104.

In some embodiments, the compounds of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196.

As provided throughout, the additional compounds, inhibitors, agents, etc. can be combined with the present compound in a single or continuous dosage form, or they can be administered simultaneously or sequentially as separate dosage forms.

### Pharmaceutical Formulations and Dosage Forms

When employed as pharmaceuticals, the compounds of the disclosure can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, the compound of the disclosure or a pharmaceutically acceptable salt thereof, in combination with one or more pharmaceutically acceptable carriers (excipients). In some embodiments, the composition is suitable for topical administration. In making the compositions of the disclosure, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

The compositions can be formulated in a unit dosage form, each dosage containing from about 5 to about 1000 mg (1 g), or more, such as about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions of the disclosure contain from about 5 to about 50 mg of the active ingredient. One having ordinary skill in the art will appreciate that this embodies compositions containing about 5 to about 10, about 10 to about 15, about 15 to about 20, about 20 to about 25, about 25 to about 30, about 30 to about 35, about 35 to about 40, about 40 to about 45, or about 45 to about 50 mg of the active ingredient.

In some embodiments, the compositions of the disclosure contain from about 50 to about 500 mg of the active ingredient. One having ordinary skill in the art will appreciate that this embodies compositions containing about 50 to about 100, about 100 to about 150, about 150 to about 200, about 200 to about 250, about 250 to about 300, about 350 to about 400, or about 450 to about 500 mg of the active ingredient.

In some embodiments, the compositions of the disclosure contain from about 500 to about 1000 mg of the active ingredient. One having ordinary skill in the art will appreciate that this embodies compositions containing about 500 to about 550, about 550 to about 600, about 600 to about 650, about 650 to about 700, about 700 to about 750, about 750 to about 800, about 800 to about 850, about 850 to about 900, about 900 to about 950, or about 950 to about 1000 mg of the active ingredient.

Similar dosages may be used of the compounds described herein in the methods and uses of the disclosure.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound of the disclosure. The topical formulations can be suitably packaged in tubes of, for example, 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present disclosure can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the disclosure can further include one or more additional pharmaceutical agents such as a chemotherapeutic, steroid, anti-inflammatory compound, or immunosuppressant, examples of which are listed herein.

### Labeled Compounds and Assay Methods

Another aspect of the present disclosure relates to labeled compounds of the disclosure (radio-labeled, fluorescent-labeled, etc.) that would be useful not only in imaging techniques but also in assays, both in vitro and in vivo, for localizing and quantitating CDK4 in tissue samples, including human, and for identifying CDK4 activators by inhibition binding of a labeled compound. Substitution of one or more of the atoms of the compounds of the present disclosure can also be useful in generating differentiated ADME (Adsorption, Distribution, Metabolism and Excretion.) Accordingly, the present disclosure includes CDK4 assays that contain such labeled or substituted compounds.

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆ alkyl group of Formula (I) can be optionally substituted with deuterium atoms, such as —CD₃ being substituted for —CH₃). In some embodiments, alkyl groups of the disclosed Formulas (e.g., Formula (I)) can be perdeuterated.

One or more constituent atoms of the compounds presented herein can be replaced or substituted with isotopes of the atoms in natural or non-natural abundance. In some embodiments, the compound includes at least one deuterium atom. For example, one or more hydrogen atoms in a compound presented herein can be replaced or substituted by deuterium (e.g., one or more hydrogen atoms of a C₁₋₆ alkyl group can be replaced by deuterium atoms, such as —CD₃ being substituted for —CH₃). In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms.

In some embodiments, 1, 2, 3, 4, 5, 6, 7, or 8 hydrogen atoms, attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene and alkynylene linking groups, as described herein, are optionally replaced by deuterium atoms.

Synthetic methods for including isotopes into organic compounds are known in the art (Deuterium Labeling in Organic Chemistry by Alan F. Thomas, New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et al. J. Med. Chem. 2011, 54, 201-210; R. Xu et al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro CDK4 labeling and competition assays, compounds that incorporate ³H, ¹⁴C, ⁸²Br, ¹²⁵I, ¹³¹I, or ³⁵S can be useful. For radio-imaging applications ¹¹C, ¹⁸F, ¹²⁵I, ¹²³I, ¹²⁴I, ¹³¹I, ⁷⁵Br, ⁷⁶Br, or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S, and ⁸²Br.

The present disclosure can further include synthetic methods for incorporating radio-isotopes into compounds of the disclosure. Synthetic methods for incorporating radio-isotopes into organic compounds are well known in the art, and one of ordinary skill in the art will readily recognize the methods applicable for the compounds of disclosure.

A labeled compound of the disclosure can be used in a screening assay to identify/evaluate compounds. For example, a newly synthesized or identified compound (i.e., test compound) which is labeled can be evaluated for its ability to bind and activate CDK4 by monitoring its concentration variation when contacting with CDK4, through tracking of the labeling. For example, a test compound (labeled) can be evaluated for its ability to reduce binding of another compound which is known to inhibit CDK4 (i.e., standard compound). Accordingly, the ability of a test compound to compete with the standard compound for binding to CDK4 directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### Kits

The present disclosure also includes pharmaceutical kits useful, for example, in the treatment or prevention of CDK4-associated diseases or disorders (such as, e.g., cancer, an inflammatory disease, a cardiovascular disease, or a neurodegenerative disease) which include one or more containers containing a pharmaceutical composition comprising a therapeutically effective amount of a compound of the disclosure. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### EXAMPLES

Experimental procedures for compounds of the invention are provided below. Preparatory LC-MS purifications of some of the compounds prepared were performed on Waters mass directed fractionation systems. The basic equipment setup, protocols, and control software for the operation of these systems have been described in detail in the literature. See e.g., “Two-Pump at-Column Dilution Configuration for Preparative LC-MS,” K. Blom, J. Combi. Chem., 4, 295 (2002); “Optimizing Preparative LC-MS Configurations and Methods for Parallel Synthesis Purification,” K. Blom, R. Sparks, J. Doughty, G. Everlof, T. Haque, A. Combs, J. Combi. Chem., 5, 670 (2003); and “Preparative LC-MS Purification: Improved Compound Specific Method Optimization,” K. Blom, B. Glass, R. Sparks, A. Combs, J. Combi. Chem., 6, 874-883 (2004). The separated compounds were typically subjected to analytical liquid chromatography mass spectrometry (LCMS) for purity check under the following conditions: Instrument: Agilent 1100 series, LC/MSD; Column: Waters Sunfire™ C₁₈ 5 μm particle size, 2.1×5.0 mm; Buffers: mobile phase A: 0.025% TFA in water and mobile phase B: acetonitrile; gradient 2% to 80% of B in 3 minutes with flow rate 2.0 mL/minute.

Some of the compounds prepared were also separated on a preparative scale by reverse-phase high performance liquid chromatography (RP-HPLC) with MS detector or flash chromatography (silica gel) as indicated in the Examples. Typical preparative reverse-phase high performance liquid chromatography (RP-HPLC) column conditions are as follows:

pH=2 purifications: Waters Sunfire™ C₁₈ 5 μm particle size, 19×100 mm column, eluting with mobile phase A: 0.1% TFA (trifluoroacetic acid) in water and mobile phase B: acetonitrile; the flow rate was 30 mL/minute, the separating gradient was optimized for each compound using the Compound Specific Method Optimization protocol as described in the literature (see “Preparative LCMS Purification: Improved Compound Specific Method Optimization,” K. Blom, B. Glass, R. Sparks, A. Combs, J. Comb. Chem., 6, 874-883 (2004)). Typically, the flow rate used with the 30×100 mm column was 60 mL/minute.

pH=10 purifications: Waters XBridge C₁₈ 5 μm particle size, 19×100 mm column, eluting with mobile phase A: 0.15% NH₄OH in water and mobile phase B: acetonitrile; the flow rate was 30 mL/minute, the separating gradient was optimized for each compound using the Compound Specific Method Optimization protocol as described in the literature (See “Preparative LCMS Purification: Improved Compound Specific Method Optimization,” K. Blom, B. Glass, R. Sparks, A. Combs, J. Comb. Chem., 6, 874-883 (2004)). Typically, the flow rate used with 30×100 mm column was 60 mL/minute.

### Intermediate 1. (3R,4R)-4-Amino-1-(methylsulfonyl)piperidin-3-ol hydrochloride

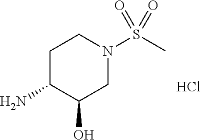

### Step 1: tert-Butyl ((3R,4R)-3-hydroxy-1-(methylsulfonyl)piperidin-4-yl)carbamate

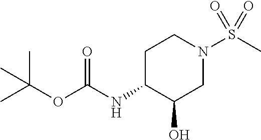 [SMILES: CC(C)(C)OC(=O)N[C@@H]1CCN(S(C)(=O)=O)C[C@H]1O]

To a mixture of tert-butyl ((3R,4R)-3-hydroxypiperidin-4-yl)carbamate (25.0 g, 116 mmol, PharmaBlock PBB1405081) and N,N-diisopropylethylamine (40.3 mL, 231 mmol) in THF (350 mL) and MeOH (17.5 mL, 433 mmol) was added methanesulfonyl chloride (9.91 mL, 127 mmol) and the reaction was stirred at r.t. for 30 min. The reaction mixture was quenched with sat. aq. NaHCO₃ (175 mL) and diluted with water (175 mL) and the resulting mixture was stirred at r.t. for 15 min. The solid precipitate that formed was collected by filtration, washed with water (350 mL), and dried to afford the desired product (27.0 g, 79% yield) as a white solid. LC-MS calculated for C₇H₁₅N₂O₅S (M+H-C₄H₈)⁺: m/z=239.1; found 239.1.

### Step 2: (3R,4R)-4-Amino-1-(methylsulfonyl)piperidin-3-ol hydrochloride

To a mixture of tert-butyl ((3R,4R)-3-hydroxy-1-(methylsulfonyl)piperidin-4-yl)carbamate (27.0 g, 92 mmol) in CH₂Cl₂/MeOH (20:1, 315 mL) was added HCl (4 M in 1,4-dioxane, 55.0 mL, 220 mmol) at r.t. The reaction mixture was stirred at 50° C. for 1 h. After cooling to r.t., the reaction mixture was diluted with Et₂O (300 mL) and slurried for 30 min. The solid precipitate was collected by filtration, washed with Et₂O (300 mL), and dried to afford the desired product (19.5 g, 92% yield) as its HCl salt in the form of a white solid. LC-MS calculated for C₆H₁₅N₂O₃S (M+H)⁺: m/z=195.1; found 195.1.

### Intermediate 2: (trans-4-Amino-1-(methylsulfonyl) piperidin-3-yl)methanol hydrochloride

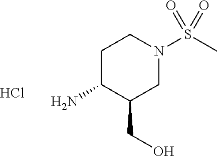

### Step 1: tert-Butyl (trans-3-(hydroxymethyl)-1-(methylsulfonyl)piperidin-4-yl)carbamate

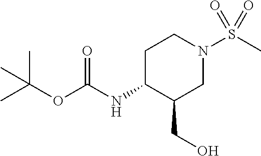 [SMILES: CC(C)(C)OC(=O)N[C@@H]1CCN(S(C)(=O)=O)C[C@H]1CO]

A mixture of tert-butyl N-(trans-3-(hydroxymethyl)piperidin-4-yl)carbamate (1.38 g, 5.99 mmol, Enamine EN300-7445487) and N-ethyl-N-isopropylpropan-2-amine (3.14 mL, 18.0 mmol) in MeCN (50.0 mL) and MeOH (1.0 mL, 25 mmol) was cooled to 0° C. before methanesulfonyl chloride (0.51 mL, 6.59 mmol) was added dropwise. The reaction mixture was allowed to warm to r.t. and stirred for 1.5 h. The mixture was quenched with saturated aqueous NaHCO₃, the organic layer was collected, and the aqueous layer was extracted with EtOAc. The combined organic layers were dried over MgSO₄, filtered, and concentrated under reduced pressure to afford a racemic mixture of the desired product as a white solid. The material crude obtained was used directly without further purification. LC-MS calculated for C₈H₁₇N₂O₅S (M-C₄H₈+H)⁺: m/z=253.1; found 253.2.

### Step 2: (trans-4-Amino-1-(methylsulfonyl)piperidin-3-yl)methanol hydrochloride

To a mixture of tert-butyl (trans-3-(hydroxymethyl)-1-(methylsulfonyl)piperidin-4-yl)carbamate (Step 1) in MeOH/CH₂Cl₂ (1:5, 6 mL) was added a HCl (4 M in 1,4-dioxane, 4.49 mL, 18.0 mmol) and the reaction mixture was and stirred at 40° C. for 1 h. After cooling to r.t., the reaction mixture was diluted with Et₂O (5 mL) and hexanes (10 mL) and slurried for 30 min. The solid precipitate was collected via filtration, washed with Et₂O and hexanes, and dried to afford a racemic mixture of the desired product (1.24 g, 85% yield) as white solid. LC-MS calculated for C₇H₁₇N₂O₃S (M+H)⁺: m/z=209.1; found 209.1.

### Intermediate 3. (3R,4R)-4-((7-Bromoquinazolin-2-yl)amino)-1-(methylsulfonyl)piperidin-3-ol

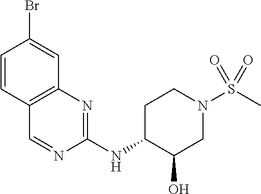 [SMILES: CS(=O)(=O)N1CC[C@@H](Nc2ncc3ccc(Br)cc3n2)[C@H](O)C1]

To a mixture of 7-bromo-2-chloroquinazoline (1.50 g, 6.16 mmol), (3R,4R)-4-amino-1-(methylsulfonyl)piperidin-3-ol hydrochloride (Intermediate 1, 2.13 g, 9.24 mmol) in MeCN (20 mL) was added N-ethyl-N-isopropylpropan-2-amine (5.7 mL, 30.8 mmol). The reaction mixture was stirred at 120° C. for 4 h. After cooling to r.t., the reaction mixture was concentrated under reduced pressure and the crude residue was purified by flash column chromatography (SiO₂, EtOAc/hexanes) to afford the product (1.80 g, 73% yield) as a yellow solid. LC-MS calculated for C₁₄H₁₈BrN₄O₃S (M+H)⁺: m/z=401.0; found 401.1.

### Intermediate 4. (3R,4R)-4-((7-Chloropyrido[3,2-d]pyrimidin-2-yl)amino)-1-(methylsulfonyl)piperidin-3-ol

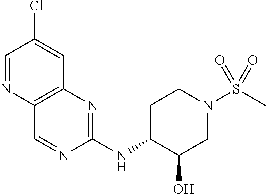 [SMILES: CS(=O)(=O)N1CC[C@@H](Nc2ncc3ncc(Cl)cc3n2)[C@H](O)C1]

The title compound was prepared according to the procedures outlined for Intermediate 3, with 2,7-dichloropyrido[3,2-d]pyrimidine replacing 7-bromo-2-chloroquinazoline. LC-MS calculated for C₁₃H₁₇ClN₅O₃S (M+H)⁺: m/z=358.1; found 358.1.

### Intermediate 5. (3R,4R)-4-((7-Chloropyrido[4,3-d]pyrimidin-2-yl)amino)-1-(methylsulfonyl)piperidin-3-ol

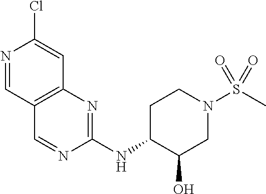 [SMILES: CS(=O)(=O)N1CC[C@@H](Nc2ncc3cnc(Cl)cc3n2)[C@H](O)C1]

The title compound was prepared according to a modification of the procedures outlined for Intermediate 3, with 2,7-dichloropyrido[4,3-d]pyrimidine replacing 7-bromo-2-chloroquinazoline. The reaction was conducted at 100° C. instead of 120° C. LC-MS calculated for C₁₃H₁₇ClN₅O₃S (M+H)⁺: m/z=358.1; found 358.1.

### Intermediate 6. (3R,4R)-4-((7-Bromo-6-chloroquinazolin-2-yl)amino)-1-(methylsulfonyl)piperidin-3-ol

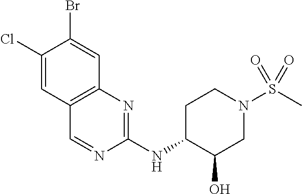 [SMILES: CS(=O)(=O)N1CC[C@@H](Nc2ncc3cc(Cl)c(Br)cc3n2)[C@H](O)C1]

The title compound was prepared according to the procedures outlined for Intermediate 3, with 7-bromo-2,6-dichloroquinazoline replacing 7-bromo-2-chloroquinazoline. LC-MS calculated for C₁₄H₁₇BrClN₄O₃S (M+H)⁺: m/z=435.0; found 435.1.

### Intermediate 7. tert-Butyl (3R,4R)-4-((7-bromoquinazolin-2-yl)amino)-3-hydroxypiperidine-1-carboxylate

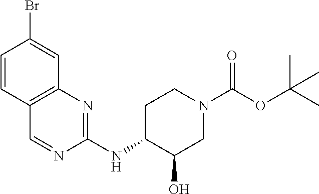 [SMILES: CC(C)(C)OC(=O)N1CC[C@@H](Nc2ncc3ccc(Br)cc3n2)[C@H](O)C1]

The title compound was prepared according to the procedures outlined for Intermediate 3, with tert-butyl (3R,4R)-4-amino-3-hydroxypiperidine-1-carboxylate (PharmaBlock PB07410) replacing (3R,4R)-4-amino-1-(methylsulfonyl)piperidin-3-ol hydrochloride (Intermediate 1). LC-MS calculated for C₁₈H₂₄BrN₄O₃ (M+H)⁺: m/z=423.1; found 423.1.

### Intermediate 8. (3R,4R)-4-((7-(1H-Pyrazol-4-yl)quinazolin-2-yl)amino)piperidin-3-ol hydrochloride

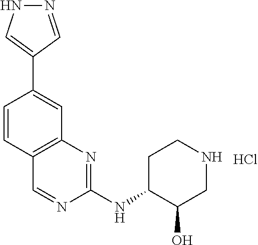 [SMILES: O[C@@H]1CNCC[C@H]1Nc1ncc2ccc(-c3cn[nH]c3)cc2n1 ; Cl]

### Step 1: tert-Butyl (3R,4R)-4-((7-(1-(1-ethoxyethyl)-1H-pyrazol-4-yl)quinazolin-2-yl)amino)-3-hydroxypiperidine-1-carboxylate

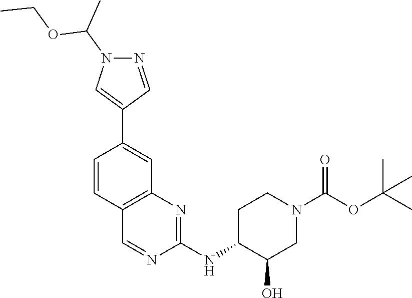 [SMILES: CCOC(C)n1cc(-c2ccc3cnc(N[C@@H]4CCN(C(=O)OC(C)(C)C)C[C@H]4O)nc3c2)cn1]

To a mixture of tert-butyl (3R,4R)-4-((7-bromoquinazolin-2-yl)amino)-3-hydroxypiperidine-1-carboxylate (1.00 g, 2.36 mmol) (Intermediate 7) in 1,4-dioxane (16 mL) and H₂O (4.0 mL) was added 1-(1-ethoxyethyl)-4-(4,4,5,5-tetramethyl-1,3,2-dioxaborolan-2-yl)-1H-pyrazole (1.25 g, 4.72 mmol), chloro(2-dicyclohexylphosphino-2′,4′,6′-triisopropyl-1,1′-biphenyl)[2-(2′-amino-1,1′-biphenyl)]palladium(II) (XPhos Pd G2, 0.175 g, 0.236 mmol), and cesium carbonate (1.54 g, 4.72 mmol). The reaction mixture was stirred at 100° C. for 1 h. After cooling to r.t., the reaction mixture was diluted with H₂O (20 mL) and extracted with EtOAc (50 mL). The combined organic layers were dried over Na₂SO₄, concentrated, and the crude residue was purified by flash column chromatography (SiO₂, EtOAc/CH₂Cl₂) to afford the desired product (1.10 g, 96% yield) as a light-yellow solid. LC-MS calculated for C₂₅H₃₅N₆O₄ (M+H)⁺: m/z=483.3; found 483.3.

### Step 2: (3R,4R)-4-((7-(1H-Pyrazol-4-yl)quinazolin-2-yl)amino)piperidin-3-ol hydrochloride

A mixture of tert-butyl (3R,4R)-4-((7-(1-(1-ethoxyethyl)-1H-pyrazol-4-yl)quinazolin-2-yl)amino)-3-hydroxypiperidine-1-carboxylate (1.10 g, 2.28 mmol) in MeOH (20 mL) was added HCl (4 M in 1,4-dioxane, 5.0 mL, 20 mmol). The mixture was stirred at 40° C. for 30 min. After cooling to r.t., the reaction mixture was concentrated under reduced pressure. Et₂O (100 mL) and hexanes (100 mL) were added and the mixture was slurried at r.t. for 15 min. The solid precipitate was collected by filtration and dried to give the desired product (0.65 g, 79% yield) as its HCl salt in the form of a light-yellow solid. LC-MS calculated for C₁₆H₁₉N₆O (M+H)⁺: m/z=311.2; found 311.2.

### Intermediate 9. (3R,4R)-4-((7-(3-Methyl-1H-pyrazol-4-yl)quinazolin-2-yl)amino)piperidin-3-ol hydrochloride

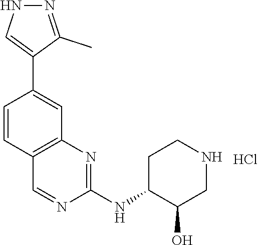 [SMILES: Cc1n[nH]cc1-c1ccc2cnc(N[C@@H]3CCNC[C@H]3O)nc2c1 ; Cl]

The title compound was prepared according to the procedures outlined for Intermediate 8, with tert-butyl 3-methyl-4-(4,4,5,5-tetramethyl-1,3,2-dioxaborolan-2-yl)-1H-pyrazole-1-carboxylate replacing 1-(1-ethoxyethyl)-4-(4,4,5,5-tetramethyl-1,3,2-dioxaborolan-2-yl)-1H-pyrazole in Step 1. LC-MS calculated for C₁₇H₂₁N₆O (M+H)⁺: m/z=325.2; found 325.2.

### Intermediate 10. (3R,4R)-4-((7-(3-Cyclopropyl-1H-pyrazol-4-yl)quinazolin-2-yl)amino)piperidin-3-ol hydrochloride

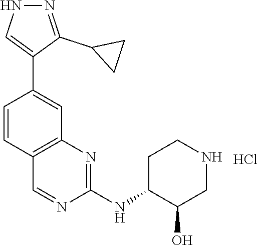 [SMILES: O[C@@H]1CNCC[C@H]1Nc1ncc2ccc(-c3c[nH]nc3C3CC3)cc2n1 ; Cl]

The title compound was prepared according to the procedures outlined for Intermediate 8, with tert-butyl 3-cyclopropyl-4-(4,4,5,5-tetramethyl-1,3,2-dioxaborolan-2-yl)-1H-pyrazole-1-carboxylate replacing 1-(1-ethoxyethyl)-4-(4,4,5,5-tetramethyl-1,3,2-dioxaborolan-2-yl)-1H-pyrazole in Step 1. LC-MS calculated for C₁₉H₂₃N₆O (M+H)⁺: m/z=351.2; found 351.2.

### Intermediate 11. (3R,4R)-1-(Methylsulfonyl)-4-((7-(4,4,5,5-tetramethyl-1,3,2-dioxaborolan-2-yl)quinazolin-2-yl)amino)piperidin-3-ol

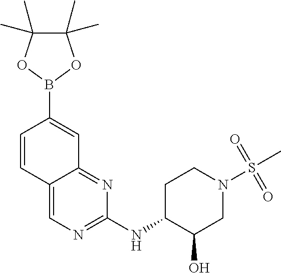

To a mixture of (3R,4R)-4-((7-bromoquinazolin-2-yl)amino)-1 (methylsulfonyl)piperidin-3-ol (Intermediate 3, 817 mg, 2.04 mmol) in THF (15 mL) was added Pd(dppf)Cl₂ (160 mg, 0.204 mmol), 4,4,4′,4′,5,5,5′,5′-octamethyl-2,2′-bi(1,3,2-dioxaborolane) (1.55 g, 6.11 mmol) and potassium acetate (599 mg, 6.11 mmol) and the reaction mixture was purged with N₂ and stirred at 100° C. for 2 h. After cooling to rt, the reaction mixture was filtered through SiO₂, the filter cake was washed with EtOAc, and the filtrate was concentrated in vacuo. The crude residue was purified by flash column chromatography (SiO₂, EtOAc/Hexanes) to afford the desired product (700 mg, 77% yield) as a white waxy solid. LC-MS calculated for C₁₄H₂₀BN₄O₅S (M+H-C₆H₁₀)⁺: m/z=367.1; found 367.1.

### Intermediate 12. (3S,4R)-4-((8-Chloro-7-(1-(1-ethoxyethyl)-1H-pyrazol-4-yl)quinazolin-2-yl)amino)tetrahydro-2H-pyran-3-ol

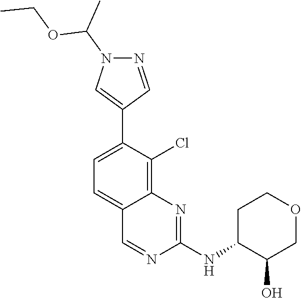 [SMILES: CCOC(C)n1cc(-c2ccc3cnc(N[C@@H]4CCOC[C@H]4O)nc3c2Cl)cn1]

### Step 1: 3-Chloro-4-(1-(1-ethoxyethyl)-1H-pyrazol-4-yl)-2-fluorobenzaldehyde

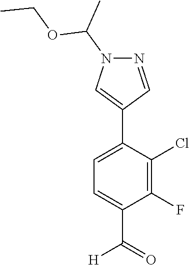 [SMILES: CCOC(C)n1cc(-c2ccc(C=O)c(F)c2Cl)cn1]

To a mixture of 1-(1-ethoxyethyl)-3-(4,4,5,5-tetramethyl-1,3,2-dioxaborolan-2-yl)-1H-pyrazole (18.5 g, 69.5 mmol), potassium carbonate (26.2 g, 190 mmol), 4-bromo-3-chloro-2-fluorobenzaldehyde (15.0 g, 63.2 mmol), and 1,1′-bis(diphenylphosphino)ferrocene-palladium(II)dichloride dichloromethane complex (5.16 g, 6.32 mmol) was charged with 1,4-dioxane (100 mL) and H₂O (20 mL). The resulting mixture was stirred at 80° C. for 18 h. After cooling to r.t., the reaction mixture was diluted with H₂O and extracted with EtOAc. The combined organic layers were dried over Na₂SO₄, concentrated, and the crude residue was purified by flash column chromatography (SiO₂, MeOH/CH₂Cl₂) to afford the product (15.3 g, 82% yield) as a light brown solid. LC-MS calculated for C₁₄H₁₅ClFN₂O₂(M+H)⁺: m/z=297.1; found 297.1.

### Step 2: 8-Chloro-7-(1-(1-ethoxyethyl)-1H-pyrazol-4-yl)quinazolin-2-amine

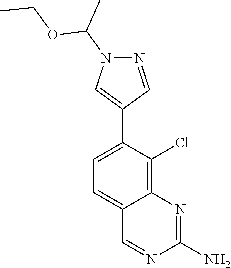 [SMILES: CCOC(C)n1cc(-c2ccc3cnc(N)nc3c2Cl)cn1]

To a mixture of 3-chloro-4-(1-(1-ethoxyethyl)-1H-pyrazol-4-yl)-2-fluorobenzaldehyde (15.3 g, 51.6 mmol), guanidine hydrochloride (4.93 g, 51.6 mmol) and potassium carbonate (14.3 g, 103 mmol) in dimethylacetamide (60.7 mL) was stirred at 150° C. for 5 h. After cooling to r.t., the reaction mixture was diluted with EtOAc and washed with saturated aqueous NaHCO₃. The layers were separated and the aqueous layer was extracted with EtOAc. The combined organic layers were washed with aqueous 5% LiCl solution, dried over MgSO₄, filtered, and concentrated under reduced pressure. The crude residue was purified by flash column chromatography (SiO₂, MeOH/CH₂Cl₂) to afford the product (3.07 g, 19% yield) as a light brown solid. LC-MS calculated for C₁₅H₁₇ClN₅O (M+H)⁺: m/z=318.1; found 318.1.

### Step 3: 2,8-Dichloro-7-(1H-pyrazol-4-yl)quinazoline

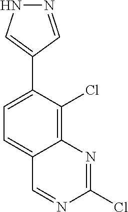 [SMILES: Clc1ncc2ccc(-c3cn[nH]c3)c(Cl)c2n1]

To a solution of 8-chloro-7-(1-(1-ethoxyethyl)-1H-pyrazol-4-yl)quinazolin-2-amine (3.07 g, 9.66 mmol), tetrabutylammonium chloride (5.37 g, 19.3 mmol), and trimethylchlorosilane (4.89 ml, 38.6 mmol) in CH₂Cl₂ (48.2 ml) and DMF (2.68 ml) was added tert-butyl nitrite (3.45 ml, 29.0 mmol) dropwise. The reaction mixture was allowed to stir at r.t for 30 min before heating to 45° C. for 3 h. The reaction mixture was diluted with EtOAc and quenched with saturated aqueous NaHCO₃. The layers were separated and the aqueous layer was extracted with EtOAc. The combined organic layers were dried over MgSO₄, filtered, and concentrated under reduced pressure. The crude residue was purified by flash column chromatography (SiO₂, MeOH/CH₂Cl₂) to afford the product (949 mg, 37% yield) as a light brown oil. LC-MS calculated for C₁₁H₇Cl₂N₄(M+H)⁺: m/z=265.0; found 265.1.

### Step 4: 2,8-Dichloro-7-(1-(1-ethoxyethyl)-1H-pyrazol-4-yl)quinazoline

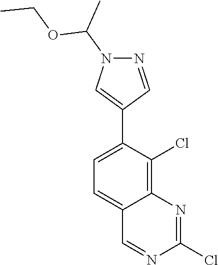 [SMILES: CCOC(C)n1cc(-c2ccc3cnc(Cl)nc3c2Cl)cn1]

A mixture of 2,2,2-trifluoroacetic acid (27.4 μL, 0.358 mmol), ethyl vinyl ether (3.43 mL, 35.8 mmol), and 2,8-dichloro-7-(1H-pyrazol-4-yl)quinazoline (949 mg, 3.58 mmol) in CH₂Cl₂ (3.58 mL) was stirred at 40° C. for 4 h. The reaction mixture was diluted with EtOAc and quenched with saturated aqueous NaHCO₃. The organic layer was dried over MgSO₄, filtered, and concentrated under reduced pressure. The resulting residue was used in the next step without further purification. LC-MS calculated for C₁₅H₁₅C₂N₄O (M+H)⁺: m/z=337.1; found 337.1.

### Step 5: (3S,4R)-4-((8-Chloro-7-(1-(1-ethoxyethyl)-1H-pyrazol-4-yl)quinazolin-2-yl)amino)tetrahydro-2H-pyran-3-ol

A mixture of (3S,4R)-4-aminotetrahydro-2H-pyran-3-ol hydrochloride (660 mg, 4.30 mmol), cesium fluoride (2.72 g, 17.9 mmol) and 2,8-dichloro-7-(1-(1-ethoxyethyl)-1H-pyrazol-4-yl)quinazoline (1.21 g, 3.58 mmol) in CH₃CN (7.16 mL) was stirred at 110° C. for 18 h. The reaction mixture was diluted with EtOAc and quenched with saturated aqueous NaHCO₃. The organic layer was dried over MgSO₄, filtered, and concentrated under reduced pressure. The crude residue was purified by flash column chromatography (SiO₂, MeOH/CH₂Cl₂) to afford the title compound as a light brown solid. LC-MS calculated for C₂₀H₂₅ClN₅O₃(M+H)⁺: m/z=418.2; found 418.2.

### Intermediate 13. 1-(2-Chloro-4-(4,4,5,5-tetramethyl-1,3,2-dioxaborolan-2-yl)phenyl)-N,N-dimethylmethanamine

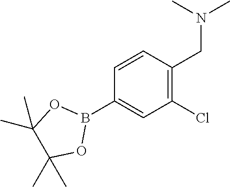

To a mixture of 2-(4-(bromomethyl)-3-chlorophenyl)-4,4,5,5-tetramethyl-1,3,2-dioxaborolane (1.00 g, 3.02 mmol) and MeOH (15 mL) was added dimethylamine (2 M in THF, 3.32 mL, 6.64 mmol). The resulting mixture was to 40° C. for 18 h. After cooling to r.t., the mixture was concentrated under reduced pressure. The crude material obtained was used directly without further purification. ¹H NMR (400 MHz, DMSO-d₆) δ 7.87-7.72 (m, 3H), 4.90 (s, 2H), 3.32 (s, 3H), 3.02 (s, 3H), 1.39-1.26 (m, 16H).

### Intermediate 14. 7-Bromo-8-isopropoxyquinazolin-2-amine

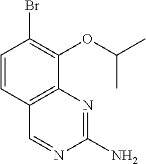 [SMILES: CC(C)Oc1c(Br)ccc2cnc(N)nc12]

To a mixture of 4-bromo-2-fluoro-3-isopropoxybenzaldehyde (2.50 g, 9.58 mmol) in DMF (10 mL) was added guanidine hydrochloride (0.915 g, 9.58 mmol) and potassium carbonate (2.65 g, 19.2 mmol). The reaction mixture was stirred at 150° C. for 5 h. After cooling to r.t., the reaction mixture was poured into a saturated solution of lithium chloride. The mixture was extracted with EtOAc, dried over Na₂SO₄, and concentrated in vacuo. The crude residue was purified by flash column chromatography (SiO₂, EtOAc/hexanes) to afford the desired product (1.2 g, 44% yield) as a white waxy solid. LC-MS calculated for C₁₁H₁₃BrN₃O (M+H)⁺: m/z=282.0; found 282.1.

### Intermediate 15. 2-Chloro-8-isopropoxy-7-(1-methyl-1H-pyrazol-4-yl)quinazoline

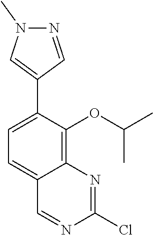 [SMILES: CC(C)Oc1c(-c2cnn(C)c2)ccc2cnc(Cl)nc12]

### Step 1. 8-Isopropoxy-7-(1-methyl-1H-pyrazol-4-yl)quinazolin-2-amine

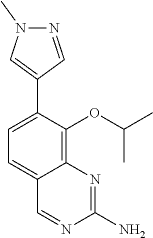 [SMILES: CC(C)Oc1c(-c2cnn(C)c2)ccc2cnc(N)nc12]

A mixture of potassium carbonate (921 mg, 6.67 mmol), 7-bromo-8-isopropoxyquinazolin-2-amine (Intermediate 8, 627 mg, 2.22 mmol), 1-methyl-3-(4,4,5,5-tetramethyl-1,3,2-dioxaborolan-2-yl)-1H-pyrazole (694 mg, 3.33 mmol) and 1,1′-bis(diphenylphosphino)ferrocene-palladium(II)dichloride dichloromethane complex (181 mg, 0.222 mmol) in 1,4-dioxane (9.26 mL) and H₂O (1.85 mL) was stirred at 80° C. for 1.5 h. After cooling to r.t., the mixture was diluted with EtOAc and washed with saturated aqueous NaHCO₃. The layers were separated and the aqueous layer was extracted with EtOAc. The combined organic layers were dried over MgSO₄, filtered, and concentrated under reduced pressure. The crude residue was purified by flash column chromatography (SiO₂, MeOH/CH₂Cl₂) to afford the desired product (550 mg, 87% yield) as a pale brown solid. LC-MS calculated for C₁₅H₁₈N₅₀ (M+H)⁺: m/z=284.2; found 284.2.

### Step 2. 2-Chloro-8-isopropoxy-7-(1-methyl-1H-pyrazol-4-yl)quinazoline

To a solution of 8-isopropoxy-7-(1-methyl-1H-pyrazol-4-yl)quinazolin-2-amine (177.5 mg, 0.626 mmol), tetrabutylammonium chloride (348 mg, 1.253 mmol), and trimethylchlorosilane (317 μL, 2.51 mmol) in CH₂Cl₂ (3.0 mL) and DMF (174 μL) was added tert-butyl nitrite (224 μL, 1.879 mmol) dropwise. The reaction mixture was allowed to stir at r.t for 30 min before heating to 45° C. for 3 h. The mixture was diluted with EtOAc and quenched with saturated aqueous NaHCO₃. The layers were separated and the aqueous layer was extracted with EtOAc. The combined organic layers were dried over MgSO₄, filtered, and concentrated under reduced pressure. The crude residue was purified by flash column chromatography (SiO₂, MeOH/CH₂Cl₂) to afford the desired product (83.9 mg, 44% yield) as a light brown oil. LC-MS calculated for C₁₅H₁₆ClN₄O (M+H)⁺: m/z=303.1; found 303.1.

### Intermediate 16. 7-Bromo-2-chloro-6,8-difluoroquinazoline

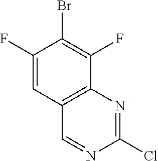 [SMILES: Fc1cc2cnc(Cl)nc2c(F)c1Br]

### Step 1. 7-Bromo-2-chloro-6,8-difluoro-3,4-dihydroquinazoline

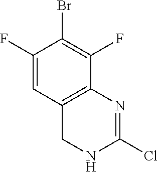 [SMILES: Fc1cc2c(c(F)c1Br)N=C(Cl)NC2]

To a mixture of 7-bromo-2,4-dichloro-6,8-difluoroquinazoline (0.50 g, 1.59 mmol) in MeOH (0.65 mL) and THF (3.33 mL) was added NaBH₄ (121 mg, 3.19 mmol) portionwise. After stirring at r.t. for 30 min, the mixture was diluted with brine and extracted with EtOAc. The combined organic layers were dried over Na₂SO₄ and concentrated under reduced pressure to give the title compound as a white solid. The crude material obtained was used directly without further purification. LC-MS calculated for C₈H₅BrClF₂N₂(M+H)⁺: m/z=280.9; found 281.0.

### Step 2. 7-Bromo-2-chloro-6,8-difluoroquinazoline

A mixture of 7-bromo-2-chloro-6,8-difluoro-3,4-dihydroquinazoline (Step 1) and manganese(IV) oxide (1.11 g, 12.7 mmol) in CH₂Cl₂ (2.0 mL) and THF (2.0 mL) was stirred at 60° C. for 30 min. After cooling to r.t., the mixture was filtered over celite, and the filter cake was washed with CH₂Cl₂. The filtrate was concentrated under reduced pressure to give the title compound as a white solid. The crude material obtained was used directly without further purification. LC-MS calculated for C₈H₃BrClF₂N₂(M+H)⁺: m/z=278.9; found 279.0.

### Intermediate 17. (3R,4R)-4-((7-Bromo-6,8-difluoroquinazolin-2-yl)amino)-1-(methylsulfonyl)piperidin-3-ol

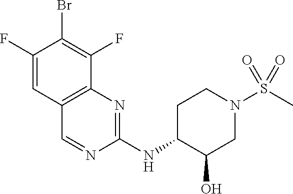 [SMILES: CS(=O)(=O)N1CC[C@@H](Nc2ncc3cc(F)c(Br)c(F)c3n2)[C@H](O)C1]

The title compound was prepared according to the procedures outlined for Intermediate 3, with 7-bromo-2-chloro-6,8-difluoroquinazoline (Intermediate 16) replacing 7-bromo-2-chloroquinazoline. LC-MS calculated for C₁₄H₁₆BrF₂N₄O₃S (M+H)⁺: m/z 437.0; found 437.1.

### Intermediate 18: (3R,4R)-4-((7-Bromoquinazolin-2-yl)amino)piperidin-3-ol hydrochloride

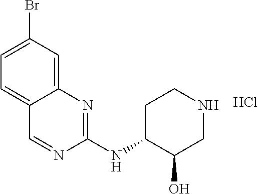 [SMILES: O[C@@H]1CNCC[C@H]1Nc1ncc2ccc(Br)cc2n1 ; Cl]

A mixture of tert-butyl (3R,4R)-4-((7-bromoquinazolin-2-yl)amino)-3-hydroxypiperidine-1-carboxylate (Intermediate 7, 2.7 g, 6.38 mmol) in MeOH (10 mL) was added HCl (4 M in 1,4-dioxane, 10 mL, 40 mmol). The mixture was stirred at 40° C. for 30 mins. After cooling to r.t., the reaction mixture was concentrated in vacuo. Et₂O (50 mL) was added and the precipitate was filtered to give desired product (2.0 g, 5.6 mmol) as a yellow solid. LC-MS calculated for C₁₃H₁₆BrN₄O (M+H)⁺: m/z=323.0; found 323.0.

### Intermediate 19: Methyl (3R,4R)-4-((7-bromoquinazolin-2-yl)amino)-3-hydroxypiperidine-1-carboxylate

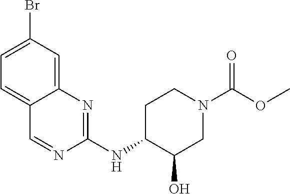 [SMILES: COC(=O)N1CC[C@@H](Nc2ncc3ccc(Br)cc3n2)[C@H](O)C1]

A mixture of (3R,4R)-4-((7-bromoquinazolin-2-yl)amino)piperidin-3-ol hydrochloride (Intermediate 18, 0.5 g, 1.39 mmol) in CH₃CN (5.0 mL) was added N-ethyl-N-isopropylpropan-2-amine (1.3 mL, 7.0 mmol), MeOH (0.1 mL, 3.1 mmol) and methyl chloroformate (0.22 mL, 2.8 mmol). The reaction mixture was stirred at r.t. for 10 min. Then reaction mixture was diluted with EtOAc (50 mL) and washed with sat. aq. NaHCO₃ (50 mL). The organic layer was dried over Na₂SO₄ and concentrated to give crude residue (0.45 g, 85% yield) as a waxy solid. LC-MS calculated for C₁₅H₁₈BrN₄O₃ (M+H)⁺: m/z=381.0; found 381.0.

### Intermediate 20: 1-((3R,4R)-4-((7-Bromoquinazolin-2-yl)amino)-3-hydroxypiperidin-1-yl)ethan-1-one

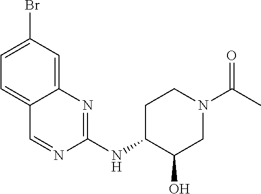 [SMILES: CC(=O)N1CC[C@@H](Nc2ncc3ccc(Br)cc3n2)[C@H](O)C1]

The title compound was prepared according to the procedures outlined for Intermediate 19, with acetic anhydride replacing methyl chloroformate. LC-MS calculated for C₁₅H₁₈BrN₄O₂ (M+H)⁺: m/z=365.1; found 365.1.

### Intermediate 21. (2-(((3R,4R)-3-Hydroxy-1-(methylsulfonyl)piperidin-4-yl)amino)quinazolin-7-yl)boronic acid

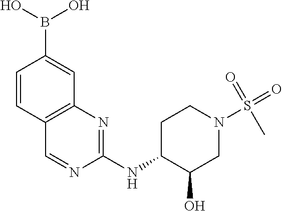 [SMILES: CS(=O)(=O)N1CC[C@@H](Nc2ncc3ccc(B(O)O)cc3n2)[C@H](O)C1]

To a mixture of (3R,4R)-4-((7-bromoquinazolin-2-yl)amino)-1-(methylsulfonyl)piperidin-3-ol (Intermediate 3, 100.0 mg, 0.25 mmol) and bis(pinacolato)diboron (95.0 mg, 0.37 mmol) in 1,4-dioxane (2 mL) was added [1,1′-bis(diphenylphosphino)ferrocene]dichloropalladium(II) (18.2 mg, 0.025 mmol) and potassium acetate (36.7 mg, 0.37 mmol). N₂ was sparged through the reaction mixture for 5 min, and the reaction mixture was stirred at 90° C. for 1 h. The reaction mixture was cooled to r.t., filtered, and concentrated. The crude residue was purified by flash column chromatography (SiO₂, MeOH/DCM) to afford the desired product. LC-MS calculated for C₁₄H₂₀BrN₄O₅S (M+H)⁺: m/z=367.3; found 367.1.

### Intermediate 22. 3-Bromo-4-iodo-1-(tetrahydro-2H-pyran-2-yl)-1H-pyrazole

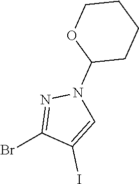 [SMILES: Brc1nn(C2CCCCO2)cc1I]

To a mixture of 3-bromo-4-iodo-1H-pyrazole (1.0 g, 3.66 mmol) and 3,4-dihydro-2H-pyran (0.67 mL, 7.33 mmol) in DCM (20 mL) was added TFA (0.028 mL, 0.37 mmol. The reaction mixture was stirred at r.t. for overnight. Then reaction mixture was diluted with EtOAc (50 mL) and washed with sat. aq. NaHCO₃ (50 mL). The organic layer was dried over Na₂SO₄ and concentrated to give crude residue, and the crude residue was purified by flash column chromatography (SiO₂, EtOAc/hexanes) to afford the desired product. LC-MS calculated for C₃H₃BrIN₂ (M+H-C₅H₈O)⁺: m/z=272.8; found 272.8.

### Intermediate 23. (3R,4R)-4-((7-(3-Bromo-1-(tetrahydro-2H-pyran-2-yl)-1H-pyrazol-4-yl)quinazolin-2-yl)amino)-1-(methylsulfonyl)piperidin-3-ol

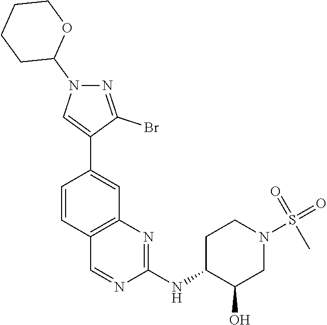 [SMILES: CS(=O)(=O)N1CC[C@@H](Nc2ncc3ccc(-c4cn(C5CCCCO5)nc4Br)cc3n2)[C@H](O)C1]

To a mixture of 3-bromo-4-iodo-1-(tetrahydro-2H-pyran-2-yl)-1H-pyrazole (Intermediate 22, 535 mg, 1.5 mmol) in 1,4-dioxane (10 mL) and H₂O (1 mL) was added (2-(((3R,4R)-3-hydroxy-1-(methylsulfonyl)piperidin-4-yl)amino)quinazolin-7-yl)boronic acid (Intermediate 21, 366 mg, 1.0 mmol), [1,1′-bis(diphenylphosphino)ferrocene]dichloropalladium(II) (73 mg, 0.1 mmol), and sodium carbonate (159 mg, 1.5 mmol). The mixture was sparged with N₂ for 5 min and stirred at 90° C. for 2 h. The reaction mixture was cooled to r.t., diluted with water and extracted with EtOAc. The combined organic layers were washed with water and brine, dried over MgSO₄, and concentrated. The crude residue was purified by flash column chromatography (SiO₂, EtOAc/hexanes) to afford the desired product (485 mg, 88%). LC-MS calculated for C₂₂H₂₈N₆O₄S (M+H)⁺: m/z=551.1; found 551.0.

### Intermediate 24. (3R,4R)-4-((7-(3-(1-Methyl-1,2,3,6-tetrahydropyridin-4-yl)-1-(tetrahydro-2H-pyran-2-yl)-1H-pyrazol-4-yl)quinazolin-2-yl)amino)-1-(methylsulfonyl)piperidin-3-ol

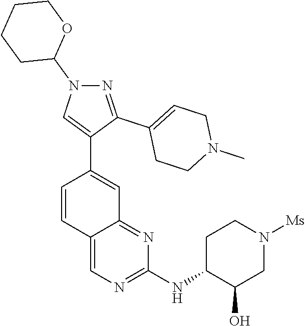 [SMILES: CN1CC=C(c2nn(C3CCCCO3)cc2-c2ccc3cnc(N[C@@H]4CCN(S(C)(=O)=O)C[C@H]4O)nc3c2)CC1]

To a mixture of (3R,4R)-4-((7-(3-bromo-1-(tetrahydro-2H-pyran-2-yl)-1H-pyrazol-4-yl)quinazolin-2-yl)amino)-1-(methylsulfonyl)piperidin-3-ol (Intermediate 23, 10.0 mg, 0.018 mmol) in 1,4-dioxane (0.5 mL) and H₂O (0.1 mL) was added 1-methyl-4-(4,4,5,5-tetramethyl-1,3,2-dioxaborolan-2-yl)-1,2,3,6-tetrahydropyridine (6.1 mg, 0.027 mmol), XPhos Pd G4 (1.6 mg, 1.81 μmol), and sodium carbonate (2.9 mg, 0.027 mmol). The reaction mixture was stirred at 90° C. for 1 h. The reaction mixture was cooled to r.t., diluted with water and extracted with EtOAc.

The combined organic layers were washed with water and brine, dried over MgSO₄, and concentrated. The crude residue was purified by flash column chromatography (SiO₂, MeOH/DCM) to afford the desired product (485 mg, 88%) LC-MS calculated for C₂₈H₃₈N₇O₄S (M+H)⁺: m/z=568.3; found 568.3.

### Intermediate 25. 4-Bromo-3-iodo-1-(tetrahydro-2H-pyran-2-yl)-1H-pyrazole

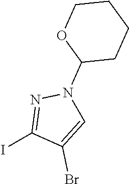 [SMILES: Brc1cn(C2CCCCO2)nc1I]

The title compound was prepared according to the procedures outlined for Intermediate 22, with 1-cyclopropyl-1H-pyrazole-3-sulfonyl chloride replacing cyclopropanesulfonyl chloride. LC-MS calculated for C₃H₃BrIN₂ (M+H-C₅H₈₀)⁺: m/z=272.8; found 272.9.

### Intermediate 26. (3R,4R)-4-Amino-1-(cyclopropylsulfonyl)piperidin-3-ol hydrochloride

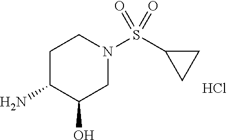 [SMILES: N[C@@H]1CCN(S(=O)(=O)C2CC2)C[C@H]1O ; Cl]

The title compound was prepared according to the procedures outlined for Intermediate 1, with cyclopropanesulfonyl chloride replacing methanesulfonyl chloride in step 1. LC-MS calculated for C₈H₁₇N₂O₃S (M+H)⁺: m/z=221.1; found 221.1.

### Intermediate 27. (3R,4R)-4-((7-Bromo-6,8-difluoroquinazolin-2-yl)amino)-1-(cyclopropylsulfonyl)piperidin-3-ol

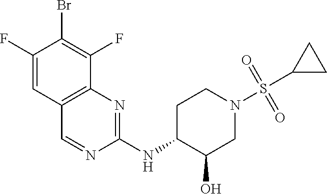 [SMILES: O=S(=O)(C1CC1)N1CC[C@@H](Nc2ncc3cc(F)c(Br)c(F)c3n2)[C@H](O)C1]

The title compound was prepared according to the procedures outlined for Intermediate 3, with (3R,4R)-4-amino-1-(cyclopropylsulfonyl)piperidin-3-ol hydrochloride (Intermediate 26) replacing (3R,4R)-4-amino-1-(methylsulfonyl)piperidin-3-ol hydrochloride (Intermediate 1) and 7-bromo-2-chloro-6,8-difluoroquinazoline replacing 7-bromo-2-chloroquinazoline. LC-MS calculated for C₁₆H₁₈BrF₂N₄O₃S (M+H)⁺: m/z=463.0; found 463.0.

### Intermediate 28. (3R,4R)-4-((7-Chloro-6-fluoroquinazolin-2-yl)amino)-1-(methylsulfonyl)piperidin-3-ol

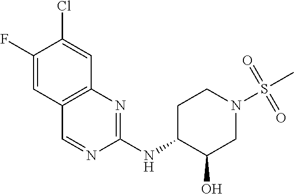 [SMILES: CS(=O)(=O)N1CC[C@@H](Nc2ncc3cc(F)c(Cl)cc3n2)[C@H](O)C1]

The title compound was prepared according to the procedures outlined for Intermediate 3, with 2,7-dichloro-6-fluoroquinazoline (PharmaBlock PBTKB0386) replacing 7-bromo-2-chloroquinazoline. LC-MS calculated for C₁₄H₁₇ClFN₄O₃S (M+H)⁺: m/z=375.1; found 375.2.

### Intermediate 29. (3R,4R)-4-((7-Chloro-6-fluoroquinazolin-2-yl)amino)-1-(cyclopropylsulfonyl)piperidin-3-ol

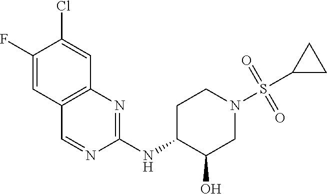 [SMILES: O=S(=O)(C1CC1)N1CC[C@@H](Nc2ncc3cc(F)c(Cl)cc3n2)[C@H](O)C1]

The title compound was prepared according to the procedures outlined for Intermediate 28, with (3R,4R)-4-amino-1-(cyclopropylsulfonyl)piperidin-3-ol hydrochloride (Intermediate 26) replacing (3R,4R)-4-amino-1-(methylsulfonyl)piperidin-3-ol hydrochloride (Intermediate 1). LC-MS calculated for C₁₆H₁₉ClFN₄O₃S (M+H)⁺: m/z=401.1; found 401.1.

### Intermediate 30. tert-Butyl (3R,4R)-4-((7-chloro-6-fluoroquinazolin-2-yl)amino)-3-hydroxypiperidine-1-carboxylate

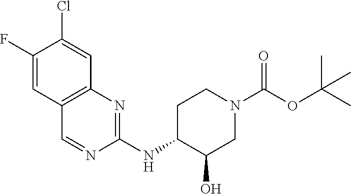 [SMILES: CC(C)(C)OC(=O)N1CC[C@@H](Nc2ncc3cc(F)c(Cl)cc3n2)[C@H](O)C1]

The title compound was prepared according to the procedures outlined for Intermediate 7, with 2,7-dichloro-6-fluoroquinazoline replacing 7-bromo-2-chloroquinazoline. LC-MS calculated for C₁₈H₂₃ClFN₄O₃(M+H)⁺: m/z=397.1; found 397.1.

### Intermediate 31. (3R,4R)-4-((6-Fluoro-7-(1-methyl-1H-imidazol-5-yl)quinazolin-2-yl)amino)piperidin-3-ol hydrochloride

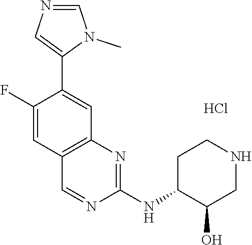 [SMILES: Cn1cncc1-c1cc2nc(N[C@@H]3CCNC[C@H]3O)ncc2cc1F ; Cl]

The title compound was prepared according to the procedures outlined for Intermediate 8, with 1-methyl-5-(4,4,5,5-tetramethyl-1,3,2-dioxaborolan-2-yl)-1H-imidazole replacing 1-(1-ethoxyethyl)-4-(4,4,5,5-tetramethyl-1,3,2-dioxaborolan-2-yl)-1H-pyrazole and tert-butyl (3R,4R)-4-((7-chloro-6-fluoroquinazolin-2-yl)amino)-3-hydroxypiperidine-1-carboxylate (Intermediate 30) replacing tert-butyl (3R,4R)-4-((7-bromoquinazolin-2-yl)amino)-3-hydroxypiperidine-1-carboxylate. LC-MS calculated for C₁₇H₂₀FN₆O (M+H)⁺: m/z=343.2; found 343.2.

### Intermediate 32. (3R,4R)-4-((7-Bromo-5-fluoroquinazolin-2-yl)amino)-1-(methylsulfonyl)piperidin-3-ol

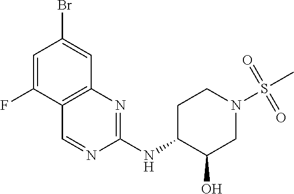 [SMILES: CS(=O)(=O)N1CC[C@@H](Nc2ncc3c(F)cc(Br)cc3n2)[C@H](O)C1]

### Step 1. 7-Bromo-2-chloro-5-fluoroquinazoline

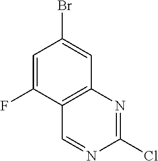 [SMILES: Fc1cc(Br)cc2nc(Cl)ncc12]

To a mixture of 7-bromo-5-fluoroquinazolin-2-amine (500.0 mg, 2.07 mmol), tetrabutylammonium chloride (1.15 g, 4.13 mmol), trimethylchlorosilane (1046 μl, 8.26 mmol), CH₂Cl₂ (196 mL), and DMF (10.9 mL) was added tert-butylnitrite (737 μL, 6.20 mmol) dropwise. The reaction mixture was allowed to stir at RT for 0.5 h and then at 45° C. for 16 h. The mixture was quenched with saturated aqueous NaHCO₃, the organic layer was collected, and the aqueous layer was extracted with EtOAc. The combined organic layers were dried over MgSO₄, filtered, and concentrated under reduced pressure. The crude residue was purified by flash column chromatography (SiO₂, EtOAc/hexanes) to afford the product (0.41 g, 76% yield) as a pale brown oil. LC-MS calculated for C₈H₄BrClFN₂ (M+H)⁺: m/z=260.0; found 260.0.

### Step 2. (3R,4R)-4-((7-Bromo-5-fluoroquinazolin-2-yl)amino)-1-(methylsulfonyl)piperidin-3-ol

The title compound was prepared according to the procedures outlined for Intermediate 7, with 7-bromo-2-chloro-5-fluoroquinazoline replacing 7-bromo-2-chloroquinazoline. LC-MS calculated for C₁₄H₁₇BrFN₄O₃S (M+H)⁺: m/z=419.0; found 419.1.

### Example 1. (3R,4R)-4-((7-(1H-Pyrazol-4-yl)quinazolin-2-yl)amino)-1 (methylsulfonyl)piperidin-3-ol

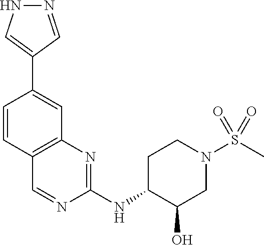 [SMILES: CS(=O)(=O)N1CC[C@@H](Nc2ncc3ccc(-c4cn[nH]c4)cc3n2)[C@H](O)C1]

A mixture of (3R,4R)-4-((7-bromoquinazolin-2-yl)amino)-1-(methylsulfonyl)piperidin-3-ol (0.150 g, 0.374 mmol) in 1,4-dioxane (4.0 mL) and H₂O (1.0 mL) was added 1-(1-ethoxyethyl)-4-(4,4,5,5-tetramethyl-1,3,2-dioxaborolan-2-yl)-1H-pyrazole (0.149 g, 0.561 mmol), chloro(2-dicyclohexylphosphino-2′,4′,6′-triisopropyl-1,1′-biphenyl)[2-(2′-amino-1,1′-biphenyl)]palladium(II) (XPhos Pd G2, 0.026 g, 0.037 mmol) and cesium carbonate (0.244 g, 0.75 mmol). The reaction mixture was stirred at 120° C. for 1 h. After cooling to r.t., the reaction mixture was treated with MeOH (5 mL) and HCl (4 M in 1,4-dioxane, 5 mL, 20 mmol) and stirred at r.t. for 30 min. The resulting mixture was filtered and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₁₇H₂₁N₆O₃S (M+H)⁺: m/z=389.1; found 389.1. ¹H NMR (500 MHz, DMSO-d₆) δ 9.18 (s, 1H), 8.29 (s, 2H), 7.90 (d, J=8.3 Hz, 1H), 7.76 (s, 1H), 7.68 (d, J=8.3 Hz, 1H), 4.12-3.86 (m, 1H), 3.71-3.62 (m, 2H), 3.61-3.51 (m, 1H), 2.94 (s, 3H), 2.91-2.82 (m, 1H), 2.75-2.62 (m, 1H), 2.18-2.02 (m, 1H), 1.72-1.56 (m, 1H).

### Example 2. (3R,4R)-4-((7-(1H-Pyrazol-4-yl)pyrido[3,2-d]pyrimidin-2-yl)amino)-1-(methylsulfonyl)piperidin-3-ol

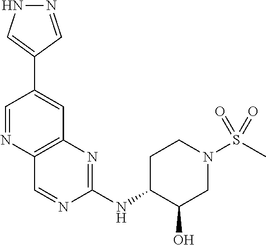 [SMILES: CS(=O)(=O)N1CC[C@@H](Nc2ncc3ncc(-c4cn[nH]c4)cc3n2)[C@H](O)C1]

The title compound was prepared according to the procedures outlined for Example 1, with (3R,4R)-4-((7-chloropyrido[3,2-d]pyrimidin-2-yl)amino)-1-(methylsulfonyl)piperidin-3-ol (Intermediate 4) replacing (3R,4R)-4-((7-bromoquinazolin-2-yl)amino)-1-(methylsulfonyl)piperidin-3-ol (Intermediate 3). LC-MS calculated for C₁₆H₂₀N₇O₃S (M+H)⁺: m/z=390.1; found 390.1.

### Example 3. (3R,4R)-4-((7-(3-Cyclopropyl-1H-pyrazol-4-yl)pyrido[4,3-d]pyrimidin-2-yl)amino)-1-(methylsulfonyl)piperidin-3-ol

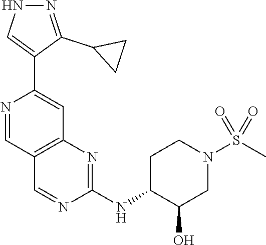 [SMILES: CS(=O)(=O)N1CC[C@@H](Nc2ncc3cnc(-c4c[nH]nc4C4CC4)cc3n2)[C@H](O)C1]

The title compound was prepared according to the procedures outlined for Example 1 with (3R,4R)-4-((7-chloropyrido[4,3-d]pyrimidin-2-yl)amino)-1-(methylsulfonyl)piperidin-3-ol (Intermediate 5) replacing (3R,4R)-4-((7-bromoquinazolin-2-yl)amino)-1-(methylsulfonyl)piperidin-3-ol (Intermediate 3) and tert-butyl 3-cyclopropyl-4-(4,4,5,5-tetramethyl-1,3,2-dioxaborola0n-2-yl)-1H-pyrazole-1-carboxylate replacing 1-(1-ethoxyethyl)-4-(4,4,5,5-tetramethyl-1,3,2-dioxaborolan-2-yl)-1H-pyrazole. The reaction mixture was heated to 100° C., instead of 120° C. LC-MS calculated for C₁₉H₂₄N₇O₃S (M+H)⁺: m/z=430.2.; found 430.2.

As one skilled in the art would understand, compounds of the present disclosure can exist as tautomers. For example, Example 3 can exist as either (3R,4R)-4-((7-(3-cyclopropyl-1H-pyrazol-4-yl)pyrido[4,3-d]pyrimidin-2-yl)amino)-1-(methylsulfonyl)piperidin-3-ol or (3R,4R)-4-((7-(5-cyclopropyl-1H-pyrazol-4-yl)pyrido[4,3-d]pyrimidin-2-yl)amino)-1-(methylsulfonyl)piperidin-3-ol.

### Example 4. (3R,4R)-4-((6-Chloro-7-(1H-pyrazol-4-yl)quinazolin-2-yl)amino)-1-(methylsulfonyl)piperidin-3-ol

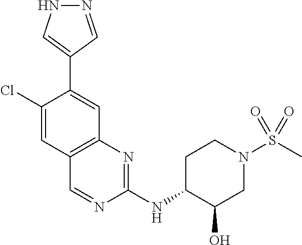 [SMILES: CS(=O)(=O)N1CC[C@@H](Nc2ncc3cc(Cl)c(-c4cn[nH]c4)cc3n2)[C@H](O)C1]

The title compound was prepared according to a modification of the procedures outlined for Example 1 with (3R,4R)-4-((7-bromo-6-chloroquinazolin-2-yl)amino)-1-(methylsulfonyl)piperidin-3-ol (Intermediate 6) replacing (3R,4R)-4-((7-bromoquinazolin-2-yl)amino)-1-(methylsulfonyl)piperidin-3-ol (Intermediate 3). The reaction was conducted at 100° C. instead of 120° C. LC-MS calculated for C₁₇H₂₀ClN₆O₃S (M+H)⁺: m/z=423.1.; found 423.1.

### Example 5. (3R,4R)-4-((7-(3-Methyl-1H-pyrazol-4-yl)quinazolin-2-yl)amino)-1-(methylsulfonyl)piperidin-3-ol

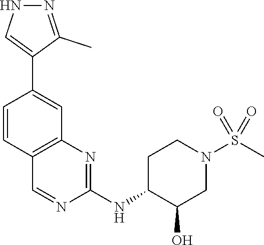 [SMILES: Cc1n[nH]cc1-c1ccc2cnc(N[C@@H]3CCN(S(C)(=O)=O)C[C@H]3O)nc2c1]

The title compound was prepared according to the procedures outlined for Example 1, with tert-butyl 3-methyl-4-(4,4,5,5-tetramethyl-1,3,2-dioxaborolan-2-yl)-1H-pyrazole-1-carboxylate) replacing 1-(1-ethoxyethyl)-4-(4,4,5,5-tetramethyl-1,3,2-dioxaborolan-2-yl)-1H-pyrazole. LC-MS calculated for C₁₈H₂₃N₆O₃S (M+H)⁺: m/z=403.1; found 403.1. ¹H NMR (500 MHz, DMSO-d₆) δ 9.19 (s, 1H), 7.98 (s, 2H), 7.90 (d, J=8.3 Hz, 1H), 7.60 (s, 1H), 7.53 (d, J=8.3 Hz, 1H), 4.03-3.98 (m, 1H), 3.72-3.64 (m, 2H), 3.63-3.55 (m, 1H), 2.97-2.88 (m, 4H), 2.79-2.70 (m, 1H), 2.48 (s, 3H), 2.20-2.11 (m, 1H), 1.71-1.61 (m, 1H).

### Example 6. 4-(2-(((3R,4R)-3-Hydroxy-1-(methylsulfonyl)piperidin-4-yl)amino)quinazolin-7-yl)pyridin-2(1H)-one

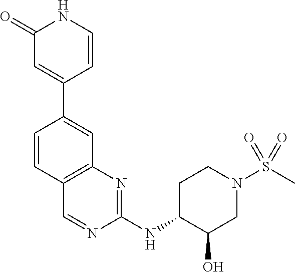 [SMILES: CS(=O)(=O)N1CC[C@@H](Nc2ncc3ccc(-c4cc[nH]c(=O)c4)cc3n2)[C@H](O)C1]

The title compound was prepared according to the procedures outlined for Example 1, with 4-(4,4,5,5-tetramethyl-1,3,2-dioxaborolan-2-yl)pyridin-2(1H)-one replacing 1-(1-ethoxyethyl)-4-(4,4,5,5-tetramethyl-1,3,2-dioxaborolan-2-yl)-1H-pyrazole. LC-MS calculated for C₁₉H₂₂N₅O₄S (M+H)⁺: m/z=416.1; found 416.1.

### Example 7. (3R,4R)-4-((7-(1H-Imidazol-4-yl)quinazolin-2-yl)amino)-1-(methylsulfonyl)piperidin-3-ol

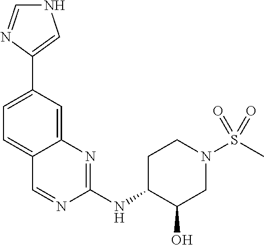 [SMILES: CS(=O)(=O)N1CC[C@@H](Nc2ncc3ccc(-c4c[nH]cn4)cc3n2)[C@H](O)C1]

The title compound was prepared according to the procedures outlined for Example 1, with N,N-dimethyl-4-(4,4,5,5-tetramethyl-1,3,2-dioxaborolan-2-yl)-1H-imidazole-1-sulfonamide replacing 1-(1-ethoxyethyl)-4-(4,4,5,5-tetramethyl-1,3,2-dioxaborolan-2-yl)-1H-pyrazole. LC-MS calculated for C₁₇H₂₁N₆O₃S (M+H)⁺: m/z=389.1; found 389.1.

### Example 8. (3R,4R)-4-((7-(1H-Pyrazol-4-yl)quinazolin-2-yl)amino)-1-((1-methyl-1H-pyrazol-4-yl)sulfonyl)piperidin-3-ol

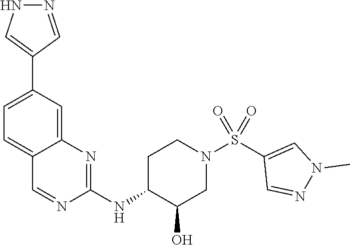 [SMILES: Cn1cc(S(=O)(=O)N2CC[C@@H](Nc3ncc4ccc(-c5cn[nH]c5)cc4n3)[C@H](O)C2)cn1]

To a mixture of (3R,4R)-4-((7-(1H-pyrazol-4-yl)quinazolin-2-yl)amino)piperidin-3-ol hydrochloride (0.120 g, 0.346 mmol) (Intermediate 8) in CH₃CN (5.0 mL) was added N-ethyl-N-isopropylpropan-2-amine (0.32 mL, 1.7 mmol), MeOH (22 μL, 0.69 mmol) and 1-methyl-1H-pyrazole-4-sulfonyl chloride (0.094 g, 0.52 mmol). The reaction mixture was stirred at r.t. for 10 min before HCl (4 M in 1,4-dioxane, 2 mL, 8.00 mmol) was added. The resulting mixture was diluted with H₂O and MeOH and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₀H₂₃N₈O₃S (M+H)⁺: m/z=455.2; found 455.2. ¹H NMR (500 MHz, DMSO-d₆) δ 9.14 (s, 1H), 8.39 (s, 1H), 8.27 (s, 2H), 7.87 (d, J=8.3 Hz, 1H), 7.83 (s, 1H), 7.71 (s, 1H), 7.65 (d, J=8.3 Hz, 1H), 3.94 (s, 3H), 3.87-3.75 (m, 1H), 3.74-3.66 (m, 1H), 3.65-3.58 (m, 1H), 3.58-3.49 (m, 1H), 2.43-2.32 (m, 1H), 2.23-2.13 (m, 1H), 2.12-2.04 (m, 1H), 1.72-1.59 (m, 1H).

### Example 9. (3R,4R)-4-((7-(3-Methyl-1H-pyrazol-4-yl)quinazolin-2-yl)amino)-1-((1-methyl-1H-pyrazol-4-yl)sulfonyl)piperidin-3-ol

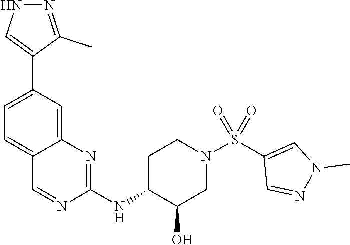 [SMILES: Cc1n[nH]cc1-c1ccc2cnc(N[C@@H]3CCN(S(=O)(=O)c4cnn(C)c4)C[C@H]3O)nc2c1]

The title compound was prepared according to the procedures outlined for Example 8, with (3R,4R)-4-((7-(3-methyl-1H-pyrazol-4-yl)quinazolin-2-yl)amino)piperidin-3-ol hydrochloride (Intermediate 9) replacing (3R,4R)-4-((7-(1H-pyrazol-4-yl)quinazolin-2-yl)amino)piperidin-3-ol hydrochloride (Intermediate 8). LC-MS calculated for C₂₁H₂₅N₈O₃S (M+H)⁺: m/z=469.2; found 469.2. ¹H NMR (500 MHz, DMSO-d₆) δ 9.25 (s, 1H), 8.39 (s, 1H), 8.04 (s, 1H), 7.95 (d, J=8.3 Hz, 1H), 7.83 (s, 1H), 7.71-7.55 (m, 2H), 3.93 (s, 3H), 3.88-3.74 (m, 1H), 3.74-3.67 (m, 1H), 3.66-3.60 (m, 1H), 3.60-3.51 (m, 1H), 2.47 (s, 3H), 2.44-2.29 (m, 1H), 2.22-2.14 (m, 1H), 2.13-2.02 (m, 1H), 1.76-1.63 (m, 1H).

### Example 10. (3R,4R)-1-(Cyclopropylsulfonyl)-4-((7-(3-methyl-1H-pyrazol-4-yl)quinazolin-2-yl)amino)piperidin-3-ol

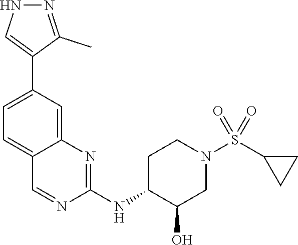 [SMILES: Cc1n[nH]cc1-c1ccc2cnc(N[C@@H]3CCN(S(=O)(=O)C4CC4)C[C@H]3O)nc2c1]

To a mixture of (3R,4R)-4-((7-(3-methyl-1H-pyrazol-4-yl)quinazolin-2-yl)amino)piperidin-3-ol hydrochloride (Intermediate 9, 0.06 g, 0.166 mmol) in CH₃CN (5.0 mL) was added N-ethyl-N-isopropylpropan-2-amine (0.15 mL, 0.83 mmol), MeOH (0.01 mL, 0.33 mmol) and cyclopropanesulfonyl chloride (34 μL, 0.33 mmol). The reaction mixture was stirred at r.t. for 10 min before HCl (4 M in 1,4-dioxane, 2.0 mL, 8.0 mmol) was added. The resulting mixture was diluted with H₂O and MeOH and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₀H₂₅N₆O₃S (M+H)⁺: m/z=429.2; found 429.2. ¹H NMR (500 MHz, DMSO-d₆) δ 9.28 (s, 1H), 8.06 (s, 1H), 7.97 (d, J=8.3 Hz, 1H), 7.71 (s, 1H), 7.62 (d, J=8.3 Hz, 1H), 4.14-3.94 (m, 1H), 3.82-3.67 (m, 1H), 3.66-3.58 (m, 2H), 3.05-2.91 (m, 1H), 2.84-2.75 (m, 1H), 2.71-2.60 (m, 1H), 2.49 (s, 3H), 2.15-2.04 (m, 1H), 1.74-1.60 (m, 1H), 1.09-0.98 (m, 2H), 0.99-0.86 (m, 2H).

### Example 11. (3R,4R)-1-((3-Methoxy-1-methyl-1H-pyrazol-4-yl)sulfonyl)-4-((7-(3-methyl-1H-pyrazol-4-yl)quinazolin-2-yl)amino)piperidin-3-ol

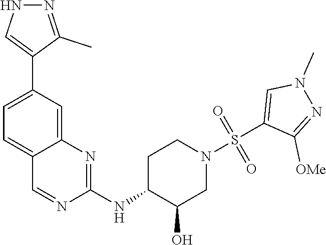 [SMILES: COc1nn(C)cc1S(=O)(=O)N1CC[C@@H](Nc2ncc3ccc(-c4c[nH]nc4C)cc3n2)[C@H](O)C1]

The title compound was prepared according to the procedures outlined for Example 10, with 3-methoxy-1-methyl-1H-pyrazole-4-sulfonyl chloride replacing cyclopropanesulfonyl chloride. LC-MS calculated for C₂₂H₂₇N₈O₄S (M+H)⁺: m/z=499.2; found 499.2. ¹H NMR (500 MHz, DMSO-d₆) δ 9.23 (s, 1H), 8.11 (s, 1H), 8.03 (s, 1H), 7.93 (d, J=8.4 Hz, 1H), 7.62 (s, 1H), 7.58 (d, J=8.4 Hz, 1H), 3.87 (s, 3H), 3.86-3.80 (m, 1H), 3.76 (s, 3H), 3.69-3.62 (m, 2H), 3.59-3.52 (m, 1H), 2.53-2.51 (m, 1H), 2.48 (s, 3H), 2.37-2.27 (m, 1H), 2.16-2.01 (m, 1H), 1.70-1.57 (m, 1H).

### Example 12. Methyl (3R,4R)-3-hydroxy-4-((7-(3-methyl-1H-pyrazol-4-yl)quinazolin-2-yl)amino)piperidine-1-carboxylate

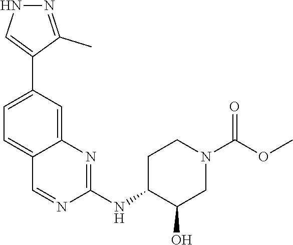 [SMILES: COC(=O)N1CC[C@@H](Nc2ncc3ccc(-c4c[nH]nc4C)cc3n2)[C@H](O)C1]

The title compound was prepared according to the procedures outlined for Example 10, with methyl chloroformate replacing cyclopropanesulfonyl chloride. LC-MS calculated for Cl₉H₂₃N₆O₃ (M+H)⁺: m/z=383.2; found 383.2. ¹H NMR (500 MHz, DMSO-d₆) δ 9.25 (s, 1H), 8.05 (s, 1H), 7.94 (d, J=8.3 Hz, 1H), 7.67 (s, 1H), 7.59 (d, J=8.4 Hz, 1H), 4.11-3.86 (m, 3H), 3.62 (s, 3H), 3.53-3.45 (m, 1H), 3.04-2.89 (m, 1H), 2.87-2.69 (m, 1H), 2.49 (s, 3H), 2.08-1.93 (m, 1H), 1.56-1.44 (m, 1H).

### Example 13. (3R,4R)-4-((7-(1H-Pyrazol-4-yl)quinazolin-2-yl)amino)-1-(cyclopropylsulfonyl)piperidin-3-ol

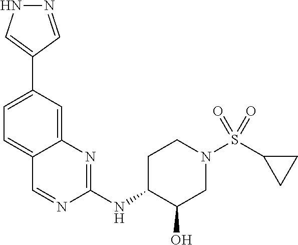 [SMILES: O=S(=O)(C1CC1)N1CC[C@@H](Nc2ncc3ccc(-c4cn[nH]c4)cc3n2)[C@H](O)C1]

The title compound was prepared according to the procedures outlined for Example 8, with cyclopropanesulfonyl chloride replacing 1-methyl-1H-pyrazole-4-sulfonyl chloride. LC-MS calculated for C₁₉H₂₃N₆O₃S (M+H)⁺: m/z=415.1; found 415.1.

### Example 14. 1-((3R,4R)-3-hydroxy-4-((7-(3-methyl-1H-pyrazol-4-yl)quinazolin-2-yl)amino)piperidin-1-yl)ethan-1-one

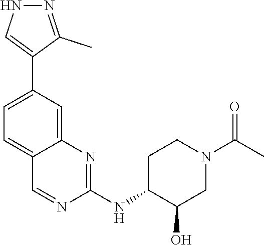 [SMILES: CC(=O)N1CC[C@@H](Nc2ncc3ccc(-c4c[nH]nc4C)cc3n2)[C@H](O)C1]

The title compound was prepared according to the procedures outlined for Example 10, with acetic anhydride replacing cyclopropanesulfonyl chloride. LC-MS calculated for C₁₉H₂₃N₆O₂ (M+H)⁺: m/z=367.2; found 367.2.

### Example 15. (3R,4R)-4-((7-(3-Cyclopropyl-1H-pyrazol-4-yl)quinazolin-2-yl)amino)-1-(pyridin-3-ylmethyl)piperidin-3-ol

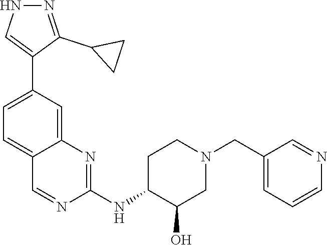 [SMILES: O[C@@H]1CN(Cc2cccnc2)CC[C@H]1Nc1ncc2ccc(-c3c[nH]nc3C3CC3)cc2n1]

To a mixture of (3R,4R)-4-((7-(3-cyclopropyl-1H-pyrazol-4-yl)quinazolin-2-yl)amino)piperidin-3-ol hydrochloride (Intermediate 10, 20.0 mg, 0.052 mmol) in THF (940 μL) and MeOH (94 μL) was added 3-(bromomethyl)pyridine hydrobromide (26.2 mg, 0.103 mmol) and N-ethyl-N-isopropylpropan-2-amine (45.0 μL, 0.258 mmol). The reaction mixture was stirred at r.t. for 30 min. The resulting mixture was diluted with MeOH and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₅H₂₈N₇₀ (M+H)⁺: m/z=442.2; found 442.3.

### Example 16. (3R,4R)-4-((7-(3-(2,2-Difluoroethoxy)-1H-pyrazol-4-yl)quinazolin-2-yl)amino)-1-(methylsulfonyl)piperidin-3-ol

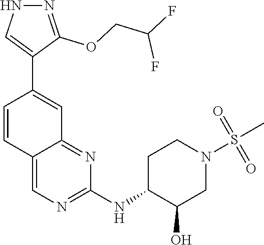 [SMILES: CS(=O)(=O)N1CC[C@@H](Nc2ncc3ccc(-c4c[nH]nc4OCC(F)F)cc3n2)[C@H](O)C1]

### Step 1: 1-(4-Bromo-3-(2,2-difluoroethoxy)-1H-pyrazol-1-yl)ethan-1-one

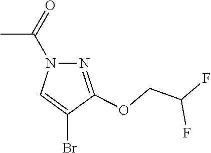

To a mixture of 1-acetyl-1,2-dihydro-3H-pyrazol-3-one (200 mg, 1.59 mmol) and potassium carbonate (565 mg, 3.17 mmol) in anhydrous DMF (2 mL) was added 1,1-difluoro-2-iodoethane (304 mg, 1.59 mmol) and the reaction mixture was stirred at 60° C. for 6 h. The reaction mixture was poured into a saturated solution of lithium chloride and extracted with EtOAc. The combined organic layers were dried over Na₂SO₄ and concentrated. The crude residue was taken up in acetonitrile (2 mL) and N-bromosuccinimide (565 mg, 3.18 mmol) was added and the reaction mixture was stirred at r.t. overnight. The reaction mixture was concentrated in vacuo and the crude residue was purified by flash column chromatography (SiO₂, EtOAc/Hexanes) to afford the desired product (300 mg, 70% yield) as a white waxy solid. LC-MS calculated for C₇H₈BrF₂N₂O₂ (M+H)⁺: m/z=269.0; found 269.0.

### Step 2: (3R,4R)-4-((7-(3-(2,2-Difluoroethoxy)-1H-pyrazol-4-yl)quinazolin-2-yl)amino)-1-(methylsulfonyl)piperidin-3-ol

To a mixture of (3R,4R)-1-(methylsulfonyl)-4-((7-(4,4,5,5-tetramethyl-1,3,2-dioxaborolan-2-yl)quinazolin-2-yl)amino)piperidin-3-ol (Intermediate 11, 39 mg, 0.086 mmol) in 1,4-dioxane (1.0 mL) and H₂O (0.2 mL) was added 1-(4-bromo-3-(2,2-difluoroethoxy)-1H-pyrazol-1-yl)ethan-1-one (23 mg, 0.086 mmol), chloro(2-dicyclohexylphosphino-2′,4′,6′-triisopropyl-1,1′-biphenyl)[2-(2′-amino-1,1′-biphenyl)]palladium(II) (XPhos Pd G2, 14 mg, 0.017 mmol) and potassium carbonate (36 mg, 0.26 mmol). The reaction mixture was stirred at 100° C. for 1 h. After cooling to r.t., the reaction mixture was treated aqueous 1 M NaOH (1 mL, 1 mmol) and stirred at r.t. for 30 min. The resulting mixture was filtered and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₁₉H₂₃F₂N₆O₄S (M+H)⁺: m/z=469.1; found 469.2.

### Example 17. (3S,4R)-4-((8-(3-Chloro-4-((dimethylamino)methyl)phenyl)-7-(1H-pyrazol-4-yl)quinazolin-2-yl)amino)tetrahydro-2H-pyran-3-ol

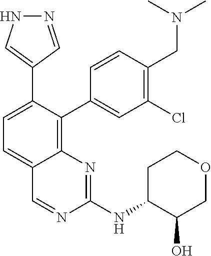 [SMILES: CN(C)Cc1ccc(-c2c(-c3cn[nH]c3)ccc3cnc(N[C@@H]4CCOC[C@H]4O)nc23)cc1Cl]

A mixture of (3S,4R)-4-((8-chloro-7-(1-(1-ethoxyethyl)-1H-pyrazol-4-yl)quinazolin-2-yl)amino)tetrahydro-2H-pyran-3-ol (Intermediate 12, 10.0 mg, 0.024 mmol), 1-(2-chloro-4-(4,4,5,5-tetramethyl-1,3,2-dioxaborolan-2-yl)phenyl)-N,N-dimethylmethanamine (Intermediate 13, 21.2 mg, 0.072 mmol), chloro(2-dicyclohexylphosphino-2′,4′,6′-triisopropyl-1,1′-biphenyl)[2-(2′-amino-1,1′-biphenyl)]palladium(II) (XPhos Pd G2, 1.88 mg, 2.39 μmol), and K₃PO₄ (25.4 mg, 0.12 mmol) in 1,4-dioxane (100 μL) and H₂O (20.0 mL) was stirred at 100° C. for 30 min. After cooling to r.t., the reaction mixture was diluted with brine and extracted with EtOAc. The combined organic layers were dried over Na₂SO₄, filtered, and concentrated under reduced pressure. The crude residue was taken up in MeOH (1 mL) and HCl (3 M in MeOH, 2.0 mL, 6 mmol) was added and the reaction mixture was stirred at r.t. for 1 h. The mixture was purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₅H₂₈ClN₆O₂(M+H)⁺: m/z=479.2; found 479.3.

### Example 18. (3S,4R)-4-((8-(4-Methylpiperazin-1-yl)-7-(1H-pyrazol-4-yl)quinazolin-2-yl)amino)tetrahydro-2H-pyran-3-ol

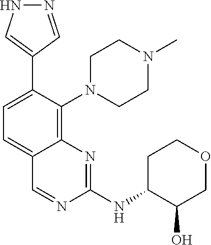 [SMILES: CN1CCN(c2c(-c3cn[nH]c3)ccc3cnc(N[C@@H]4CCOC[C@H]4O)nc23)CC1]

A mixture of (3S,4R)-4-((8-chloro-7-(1-(1-ethoxyethyl)-1H-pyrazol-4-yl)quinazolin-2-yl)amino)tetrahydro-2H-pyran-3-ol (Intermediate 12, 10.0 mg, 0.024 mmol), 1-methylpiperazine (4.8 mg, 0.048 mmol), Pd-PEPPSI™-IPent catalyst (Sigma-Aldrich 732117, 3.8 mg, 4.79 μmol), and sodium tert-butoxide (6.90 mg, 0.072 mmol) in 1,4-dioxane (479 μL) was stirred at 120° C. for 30 min. After cooling to r.t., the reaction mixture was diluted with brine and extracted with EtOAc. The combined organic layers were dried over Na₂SO₄, filtered, and concentrated. The crude residue was taken up in MeOH (1 mL) and HCl (3 M in MeOH, 2.0 mL, 6 mmol) was added and the reaction mixture was stirred at r.t. for 1 h. The mixture was purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₁H₂₈N₇O₂ (M+H)⁺: m/z=410.2; found 410.3.

### Example 19. ((3R,4R)-4-((8-Isopropoxy-7-(1-methyl-1H-pyrazol-4-yl)quinazolin-2-yl)amino)-1-(methylsulfonyl)piperidin-3-yl)methanol

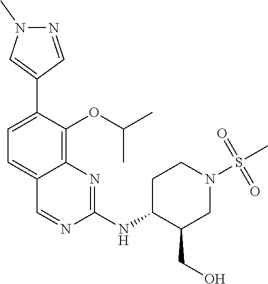

A mixture of 2-chloro-8-isopropoxy-7-(1-methyl-1H-pyrazol-4-yl)quinazoline (Intermediate 15, 10.0 mg, 0.033 mmol) and ((3R,4R)-4-amino-1-(methylsulfonyl)piperidin-3-yl)methanol hydrochloride (Intermediate 2, 9.70 mg, 0.040 mmol), cesium fluoride (25.1 mg, 0.165 mmol) in CH₃CN (661 μL) was heated at 110° C. for 3 h. After cooling to r.t., the mixture was filtered and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₂H₃₁N₆O₄S (M+H)⁺: m/z=475.3; found 475.3.

### Example 20. (3R,4R)-1-(Methylsulfonyl)-4-((7-(pyridin-3-yl)quinazolin-2-yl)amino)piperidin-3-ol

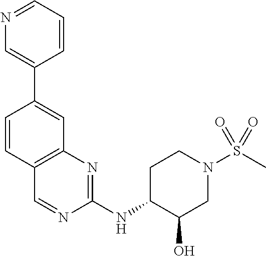 [SMILES: CS(=O)(=O)N1CC[C@@H](Nc2ncc3ccc(-c4cccnc4)cc3n2)[C@H](O)C1]

The title compound was prepared according to the procedures outlined for Example 1, with 4-(4,4,5,5-tetramethyl-1,3,2-dioxaborolan-2-yl)pyridine replacing 1-(1-ethoxyethyl)-4-(4,4,5,5-tetramethyl-1,3,2-dioxaborolan-2-yl)-1H-pyrazole. LC-MS calculated for C₁₉H₂₂N₅O₃S (M+H)⁺: m/z=400.1; found 400.1.

### Example 21. (3R,4R)-1-(Methylsulfonyl)-4-((7-(thiazol-5-yl)quinazolin-2-yl)amino)piperidin-3-ol

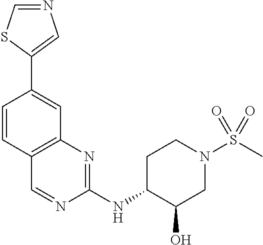 [SMILES: CS(=O)(=O)N1CC[C@@H](Nc2ncc3ccc(-c4cncs4)cc3n2)[C@H](O)C1]

The title compound was prepared according to the procedures outlined for Example 1, with 4-(4,4,5,5-tetramethyl-1,3,2-dioxaborolan-2-yl)pyridine replacing 1-(1-ethoxyethyl)-4-(4,4,5,5-tetramethyl-1,3,2-dioxaborolan-2-yl)-1H-pyrazole. LC-MS calculated for C₁₇H₂₀N₅O₃S₂ (M+H)⁺: m/z=406.1; found 406.1.

### Example 22. (3R,4R)-4-((7-(6,7-Dihydro-5H-pyrazolo[5,1-b][1,3]oxazin-3-yl)quinazolin-2-yl)amino)-1-(methylsulfonyl)piperidin-3-ol

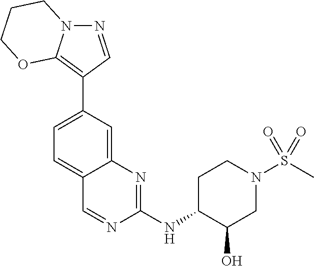 [SMILES: CS(=O)(=O)N1CC[C@@H](Nc2ncc3ccc(-c4cnn5c4OCCC5)cc3n2)[C@H](O)C1]

The title compound was prepared according to the procedures outlined for Example 1, with 3-(4,4,5,5-tetramethyl-1,3,2-dioxaborolan-2-yl)-6,7-dihydro-5H-pyrazolo[5,1-b][1,3]oxazine replacing 1-(1-ethoxyethyl)-4-(4,4,5,5-tetramethyl-1,3,2-dioxaborolan-2-yl)-1H-pyrazole. LC-MS calculated for C₂₀H₂₅N₆O₄S (M+H)⁺: m/z=445.2; found 445.2.

### Example 23. (3R,4R)-1-((3,3-Difluoroazetidin-1-yl)sulfonyl)-4-((7-(3-methyl-1H-pyrazol-4-yl)quinazolin-2-yl)amino)piperidin-3-ol

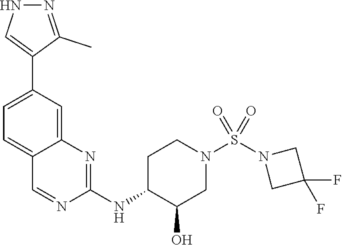 [SMILES: Cc1n[nH]cc1-c1ccc2cnc(N[C@@H]3CCN(S(=O)(=O)N4CC(F)(F)C4)C[C@H]3O)nc2c1]

The title compound was prepared according to the procedures outlined for Example 10, with 3,3-difluoroazetidine-1-sulfonyl chloride replacing cyclopropanesulfonyl chloride. LC-MS calculated for C₂₀H₂₄N₇O₃S (M+H)⁺: m/z=480.2; found 480.2.

### Example 24. (3R,4R)-4-((7-(3-Cyclopropyl-1H-pyrazol-4-yl)-6,8-difluoroquinazolin-2-yl)amino)-1-(methylsulfonyl)piperidin-3-ol

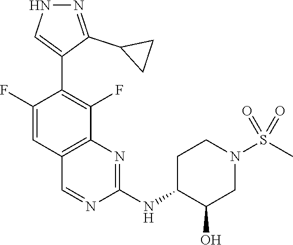 [SMILES: CS(=O)(=O)N1CC[C@@H](Nc2ncc3cc(F)c(-c4c[nH]nc4C4CC4)c(F)c3n2)[C@H](O)C1]

A mixture of tert-butyl 3-cyclopropyl-4-(4,4,5,5-tetramethyl-1,3,2-dioxaborolan-2-yl)-1H-pyrazole-1-carboxylate (30.6 mg, 0.091 mmol), potassium carbonate (19.0 mg, 0.137 mmol), (3R,4R)-4-((7-bromo-6,8-difluoroquinazolin-2-yl)amino)-1-(methylsulfonyl)piperidin-3-ol (Intermediate 17, 20.0 mg, 0.046 mmol), and 1,1′-bis(diphenylphosphino)ferrocene-palladium(II)dichloride dichloromethane complex (3.74 mg, 4.57 μmol) in 1,4-dioxane (3.81 mL) and H₂O (762 μL) was purged with N₂ for 5 min and stirred at 80° C. for 1 h. After cooling to r.t., the reaction mixture was diluted with brine and extracted with EtOAc. The combined organic layers were dried over Na₂SO₄, filtered, and concentrated. The crude residue was taken up in MeOH (1 mL) and HCl (3 M in MeOH, 2.0 mL, 6 mmol) was added and the reaction mixture was stirred at r.t. for 1 h. The mixture was purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₀H₂₃F₂N₆O₃S (M+H)⁺: m/z=465.2; found 465.2.

### Example 25. 2-((3R,4R)-4-((7-(3-Cyclopropyl-1H-pyrazol-4-yl)quinazolin-2-yl)amino)-3-hydroxypiperidin-1-yl)acetamide

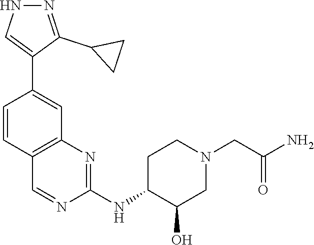 [SMILES: NC(=O)CN1CC[C@@H](Nc2ncc3ccc(-c4c[nH]nc4C4CC4)cc3n2)[C@H](O)C1]

The title compound was prepared according to the procedures outlined for Example 15, with 2-bromoacetamide replacing 3-(bromomethyl)pyridine hydrobromide. LC-MS calculated for C₂₁H₂₆N₇O₂ (M+H)⁺: m/z=408.2; found 408.3.

### Example 26. (S)-5-(((3R,4R)-4-((7-(3-Cyclopropyl-1H-pyrazol-4-yl)quinazolin-2-yl)amino)-3-hydroxypiperidin-1-yl)methyl)pyrrolidin-2-one

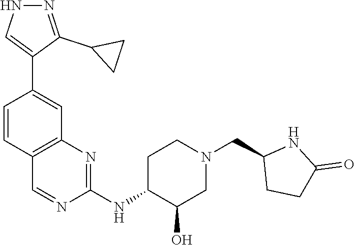 [SMILES: O=C1CC[C@@H](CN2CC[C@@H](Nc3ncc4ccc(-c5c[nH]nc5C5CC5)cc4n3)[C@H](O)C2)N1]

The title compound was prepared according to the procedures outlined for Example 15, with (S)-5-(bromomethyl)pyrrolidin-2-one replacing 3-(bromomethyl)pyridine hydrobromide. LC-MS calculated for C₂₄H₃₀N₇O₂ (M+H)⁺: m/z=448.3; found 448.3.

### Example 27. (3R,4R)-1-((1-Cyclopropyl-1H-pyrazol-3-yl)sulfonyl)-4-((7-(3-methyl-1H-pyrazol-4-yl)quinazolin-2-yl)amino)piperidin-3-ol

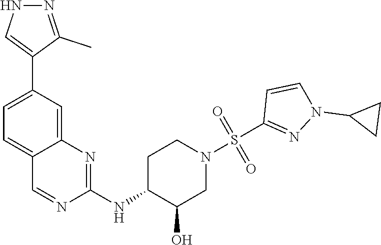 [SMILES: Cc1n[nH]cc1-c1ccc2cnc(N[C@@H]3CCN(S(=O)(=O)c4ccn(C5CC5)n4)C[C@H]3O)nc2c1]

The title compound was prepared according to the procedures outlined for Example 10, with 1-cyclopropyl-1H-pyrazole-3-sulfonyl chloride replacing cyclopropanesulfonyl chloride. LC-MS calculated for C₂₃H₂₇N₈O₃S (M+H)⁺: m/z=495.2; found 495.2. ¹H NMR (600 MHz, DMSO-d₆) δ 9.26 (s, 1H), 8.08 (d, J=2.4 Hz, 1H), 8.04 (s, 1H), 7.95 (d, J=8.3 Hz, 1H), 7.69 (s, 1H), 7.61 (d, J=8.4 Hz, 1H), 6.70 (d, J=2.4 Hz, 1H), 3.95-3.89 (m, 1H), 3.89-3.79 (m, 1H), 3.77-3.69 (m, 1H), 3.68-3.57 (m, 2H), 2.64-2.53 (m, 1H), 2.49-2.44 (m, 3H), 2.42-2.34 (m, 1H), 2.15-1.99 (m, 1H), 1.69-1.59 (m, 1H), 1.17-1.11 (m, 2H), 1.08-1.02 (m, 2H).

### Example 28. Methyl (3R,4R)-4-((7-(3-cyclopropyl-1H-pyrazol-4-yl)quinazolin-2-yl)amino)-3-hydroxypiperidine-1-carboxylate

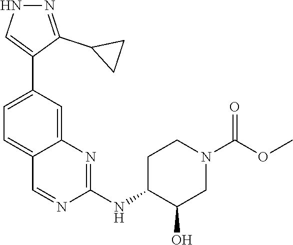 [SMILES: COC(=O)N1CC[C@@H](Nc2ncc3ccc(-c4c[nH]nc4C4CC4)cc3n2)[C@H](O)C1]

To a mixture of methyl (3R,4R)-4-((7-bromoquinazolin-2-yl)amino)-3-hydroxypiperidine-1-carboxylate (Intermediate 19, 0.1 g, 0.26 mmol) in 1,4-dioxane (5 mL) and H₂O (1 mL) was added tert-butyl 3-cyclopropyl-4-(4,4,5,5-tetramethyl-1,3,2-dioxaborolan-2-yl)-1H-pyrazole-1-carboxylate (0.17 g, 0.52 mmol), chloro(2-dicyclohexylphosphino-2′,4′,6′-triisopropyl-1,1′-biphenyl)[2-(2′-amino-1,1′-biphenyl)]palladium(II) (XPhos Pd G2, 32 mg, 0.04 mmol), and cesium carbonate (0.26 mg, 0.8 mmol). The reaction mixture was stirred at 120° C. for 1 h. After cooling to r.t., the reaction mixture was diluted with MeOH, filtered and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₁H₂₅N₆O₃ (M+H)⁺: m/z=409.2; found 409.2. ¹H NMR (600 MHz, DMSO-d₆) δ 9.28 (s, 1H), 8.06 (s, 1H), 7.98 (s, 1H), 7.89 (s, 1H), 7.74 (d, J=8.4 Hz, 1H), 4.20-3.85 (m, 3H), 3.62 (s, 3H), 3.52-3.44 (m, 1H), 3.03-2.88 (m, 1H), 2.86-2.64 (m, 1H), 2.17-1.89 (m, 2H), 1.57-1.43 (m, 1H), 1.10-0.96 (m, 2H), 0.92-0.80 (m, 2H).

### Example 29. Methyl (3R,4R)-4-((7-(3-ethyl-1H-pyrazol-4-yl)quinazolin-2-yl)amino)-3-hydroxypiperidine-1-carboxylate

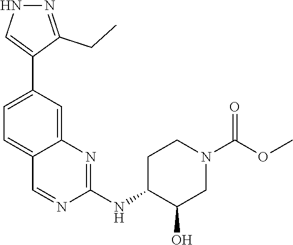 [SMILES: CCc1n[nH]cc1-c1ccc2cnc(N[C@@H]3CCN(C(=O)OC)C[C@H]3O)nc2c1]

The title compound was prepared according to the procedures outlined for Example 28, with 3-ethyl-4-(4,4,5,5-tetramethyl-1,3,2-dioxaborolan-2-yl)-1H-pyrazole replacing tert-butyl 3-cyclopropyl-4-(4,4,5,5-tetramethyl-1,3,2-dioxaborolan-2-yl)-1H-pyrazole-1-carboxylate. LC-MS calculated for C₂₀H₂₅N₆O₃ (M+H)⁺: m/z=397.2; found 397.2. ¹H NMR (600 MHz, DMSO-d₆) δ 9.30 (s, 1H), 8.03 (s, 1H), 8.01-7.92 (m, 1H), 7.74 (s, 1H), 7.65-7.55 (m, 1H), 4.08-3.90 (m, 3H), 3.62 (s, 3H), 3.53-3.44 (m, 1H), 3.03-2.86 (m, 3H), 2.82-2.67 (m, 1H), 2.08-1.92 (m, 1H), 1.57-1.46 (m, 1H), 1.28-1.21 (m, 3H).

### Example 30. 1-((3R,4R)-4-((7-(3-Cyclopropyl-1H-pyrazol-4-yl)quinazolin-2-yl)amino)-3-hydroxypiperidin-1-yl)ethan-1-one

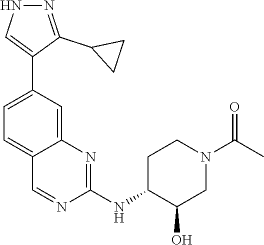 [SMILES: CC(=O)N1CC[C@@H](Nc2ncc3ccc(-c4c[nH]nc4C4CC4)cc3n2)[C@H](O)C1]

The title compound was prepared according to the procedures outlined for Example 28, with 1-((3R,4R)-4-((7-bromoquinazolin-2-yl)amino)-3-hydroxypiperidin-1-yl)ethan-1-one (Intermediate 20) replacing methyl (3R,4R)-4-((7-bromoquinazolin-2-yl)amino)-3-hydroxypiperidine-1-carboxylate (Intermediate 19). LC-MS calculated for C₂₁H₂₅N₆O₂ (M+H)⁺: m/z=393.2; found 393.2.

TABLE 1
The compounds in Table 1 were prepared in accordance with the synthetic protocols set
forth in Example 1 using the appropriate starting materials.

| | | | Analytical |
|---|---|---|---|
| Ex. | Name | Structure | data |
| 31 | (3S,4R)-4-((7-(1H- Pyrazol-4- yl)quinazolin-2- yl)amino)tetrahydro- 2H-pyran-3-ol | 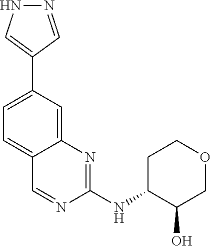 [SMILES: O[C@@H]1COCC[C@H]1Nc1ncc2ccc(-c3cn[nH]c3)cc2n1] | LCMS found 312.1 |
| 32 | (3S,4R)-4-((7-(1H- Pyrazol-4- yl)quinazolin-2- yl)amino)-1- (methylsulfonyl) piperidin-3-ol | 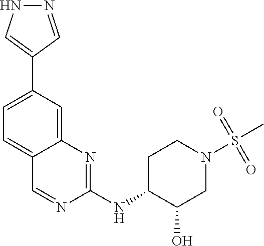 [SMILES: CS(=O)(=O)N1CC[C@@H](Nc2ncc3ccc(-c4cn[nH]c4)cc3n2)[C@@H](O)C1] | LCMS found 389.1 |
| 33 | Rac-((3R,4R)-4-((7- (1H-pyrazol-4- yl)quinazolin-2- yl)amino)-1- (methylsulfonyl) piperidin- 3-yl)methanol | 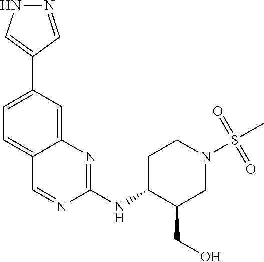 [SMILES: CS(=O)(=O)N1CC[C@@H](Nc2ncc3ccc(-c4cn[nH]c4)cc3n2)[C@H](CO)C1] | LCMS found 403.1 |
| 34 | (3R,4R)-4-((7-(3- Isopropyl-1H-pyrazol- 4-yl)quinazolin-2- yl)amino)-1- (methylsulfonyl) piperidin-3-ol | 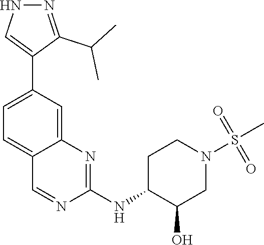 [SMILES: CC(C)c1n[nH]cc1-c1ccc2cnc(N[C@@H]3CCN(S(C)(=O)=O)C[C@H]3O)nc2c1] | LCMS found 431.1 |
| 35 | (3R,4R)-4-((7-(3- Cyclopropyl-1H- pyrazol-4- yl)quinazolin-2- yl)amino)-1- (methylsulfonyl) piperidin-3-ol | 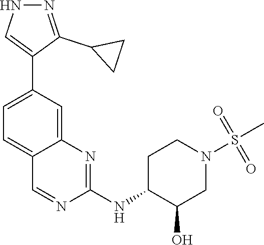 [SMILES: CS(=O)(=O)N1CC[C@@H](Nc2ncc3ccc(-c4c[nH]nc4C4CC4)cc3n2)[C@H](O)C1] | LCMS found 429.2 |
| 36 | (3R,4R)-4-((7-(3- Fluoro-1H-pyrazol-4- yl)quinazolin-2- yl)amino)-1- (methylsulfonyl) piperidin-3-ol | 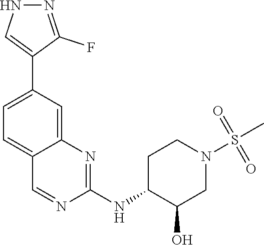 [SMILES: CS(=O)(=O)N1CC[C@@H](Nc2ncc3ccc(-c4c[nH]nc4F)cc3n2)[C@H](O)C1] | LCMS found 407.1 |
| 37 | (3R,4R)-4-((7-(3- Methoxy-1H-pyrazol- 4-yl)quinazolin-2- yl)amino)-1- (methylsulfonyl) piperidin-3-ol | 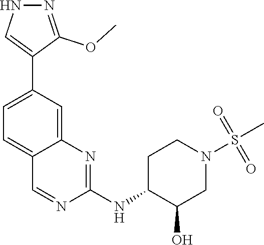 [SMILES: COc1n[nH]cc1-c1ccc2cnc(N[C@@H]3CCN(S(C)(=O)=O)C[C@H]3O)nc2c1] | LCMS found 418.1 |
| 38 | (3R,4R)-4-((7-(1- Methyl-1H-imidazol-4- yl)quinazolin-2- yl)amino)-1- (methylsulfonyl) piperidin-3-ol | 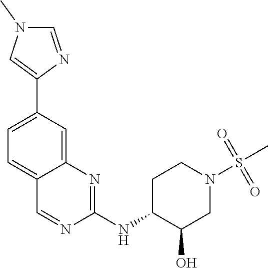 | LCMS found 403.1 |
| 39 | (3R,4R)-4-((7-(1- Methyl-1H-imidazol-5- yl)quinazolin-2- yl)amino)-1- (methylsulfonyl) piperidin-3-ol | 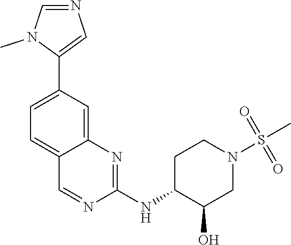 | LCMS found 403.1 |
| 40 | (3R,4R)-1- (Methylsulfonyl)-4-((7- (pyridin-4- yl)quinazolin-2- yl)amino)piperidin-3-ol | 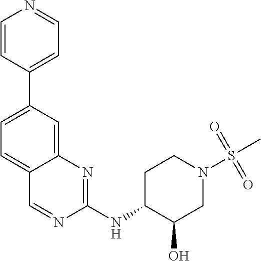 [SMILES: CS(=O)(=O)N1CC[C@@H](Nc2ncc3ccc(-c4ccncc4)cc3n2)[C@H](O)C1] | LCMS found 400.1 |
| 41 | (3R,4R)-1- (Methylsulfonyl)-4-((7- (pyridazin-4- yl)quinazolin-2- yl)amino)piperidin-3-ol | 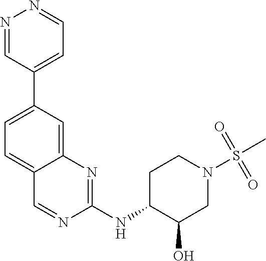 [SMILES: CS(=O)(=O)N1CC[C@@H](Nc2ncc3ccc(-c4ccnnc4)cc3n2)[C@H](O)C1] | LCMS found 401.1 |
| 42 | (3R,4R)-4-((7- (Isothiazol-4- yl)quinazolin-2- yl)amino)-1- (methylsulfonyl) piperidin-3-ol | 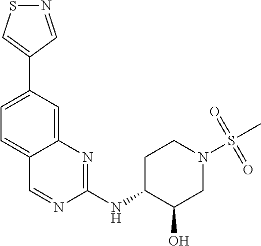 [SMILES: CS(=O)(=O)N1CC[C@@H](Nc2ncc3ccc(-c4cnsc4)cc3n2)[C@H](O)C1] | LCMS found 406.1 |
| 43 | (3R,4R)-1- (Methylsulfonyl)-4-((7- (3-(trifluoromethyl)- 1H-pyrazol-4- yl)quinazolin-2- yl)amino)piperidin-3-ol | 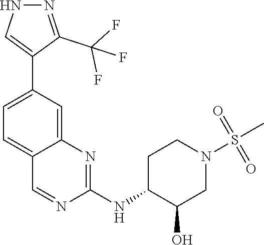 [SMILES: CS(=O)(=O)N1CC[C@@H](Nc2ncc3ccc(-c4c[nH]nc4C(F)(F)F)cc3n2)[C@H](O)C1] | LCMS found 457.1 |
| 44 | 4-(2-(((3R,4R)-3- Hydroxy-1- (methylsulfonyl) piperidin-4- yl)amino)quinazolin-7- yl)-1H-pyrazole-3- carbonitrile | 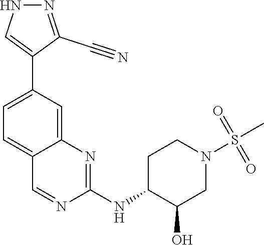 [SMILES: CS(=O)(=O)N1CC[C@@H](Nc2ncc3ccc(-c4c[nH]nc4C#N)cc3n2)[C@H](O)C1] | LCMS found 414.1 |
| 45 | (3R,4R)-4-((7-(3-Ethyl- 1H-pyrazol-4- yl)quinazolin-2- yl)amino)-1- (methylsulfonyl) piperidin-3-ol | 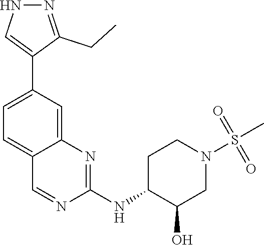 [SMILES: CCc1n[nH]cc1-c1ccc2cnc(N[C@@H]3CCN(S(C)(=O)=O)C[C@H]3O)nc2c1] | LCMS found 417.1 |
| 46 | (3R,4R)-4-((7-(1- Methyl-1H-pyrazol-5- yl)quinazolin-2- yl)amino)-1- (methylsulfonyl) piperidin-3-ol | 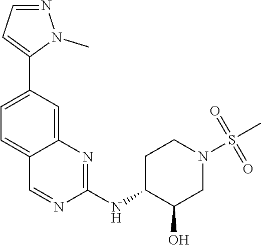 [SMILES: Cn1nccc1-c1ccc2cnc(N[C@@H]3CCN(S(C)(=O)=O)C[C@H]3O)nc2c1] | LCMS found 403.1 |
| 47 | (3R,4R)-4-((7-(1H- Pyrazol-5- yl)quinazolin-2- yl)amino)-1- (methylsulfonyl) piperidin-3-ol | 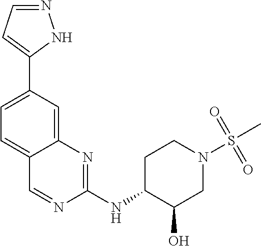 [SMILES: CS(=O)(=O)N1CC[C@@H](Nc2ncc3ccc(-c4ccn[nH]4)cc3n2)[C@H](O)C1] | LCMS found 389.1 |
| 48 | (3R,4R)-4-((7-(1- Cyclopropyl-1H- pyrazol-5- yl)quinazolin-2- yl)amino)-1- (methylsulfonyl) piperidin-3-ol | 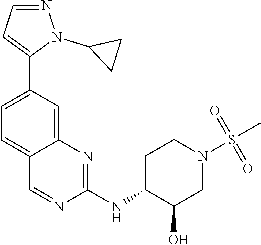 [SMILES: CS(=O)(=O)N1CC[C@@H](Nc2ncc3ccc(-c4ccnn4C4CC4)cc3n2)[C@H](O)C1] | LCMS found 429.1 |
| 49 | (3R,4R)-4-((7-(1-Ethyl- 1H-pyrazol-5- yl)quinazolin-2- yl)amino)-1- (methylsulfonyl) piperidin-3-ol | 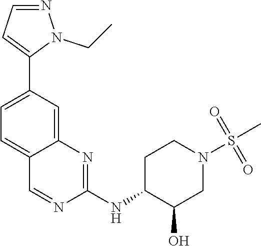 [SMILES: CCn1nccc1-c1ccc2cnc(N[C@@H]3CCN(S(C)(=O)=O)C[C@H]3O)nc2c1] | LCMS found 417.2 |
| 50 | (3R,4R)-4-((7-(1-Ethyl- 1H-imidazol-5- yl)quinazolin-2- yl)amino)-1- (methylsulfonyl) piperidin-3-ol | 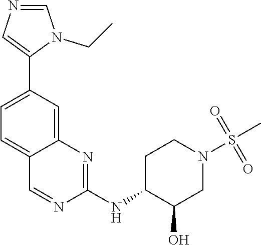 [SMILES: CCn1cncc1-c1ccc2cnc(N[C@@H]3CCN(S(C)(=O)=O)C[C@H]3O)nc2c1] | LCMS found 417.2 |
| 51 | (3R,4R)-4-((7-(1- Cyclopropyl-1H- imidazol-5- yl)quinazolin-2- yl)amino)-1- (methylsulfonyl) piperidin-3-ol | 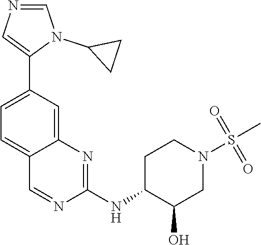 [SMILES: CS(=O)(=O)N1CC[C@@H](Nc2ncc3ccc(-c4cncn4C4CC4)cc3n2)[C@H](O)C1] | LCMS found 429.2 |
| 52 | (3R,4R)-4-((7-(3- Fluoro-1-methyl-1H- pyrazol-4- yl)quinazolin-2- yl)amino)-1- (methylsulfonyl) piperidin-3-ol | 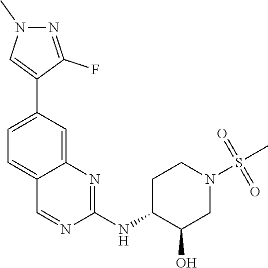 | LCMS found 421.1 |
| 53 | (3R,4R)-4-((7-(5- Fluoro-1-methyl-1H- pyrazol-4- yl)quinazolin-2- yl)amino)-1- (methylsulfonyl) piperidin-3-ol | 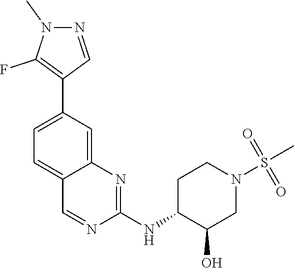 [SMILES: Cn1ncc(-c2ccc3cnc(N[C@@H]4CCN(S(C)(=O)=O)C[C@H]4O)nc3c2)c1F] | LCMS found 421.1 |
| 54 | (3R,4R)-1- (Methylsulfonyl)-4-((7- (3-(tetrahydro-2H- pyran-4-yl)-1H- pyrazol-4- yl)quinazolin-2- yl)amino)piperidin-3-ol | 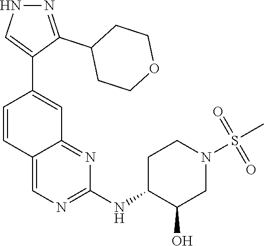 [SMILES: CS(=O)(=O)N1CC[C@@H](Nc2ncc3ccc(-c4c[nH]nc4C4CCOCC4)cc3n2)[C@H](O)C1] | LCMS found 473.2 |
| 55 | (3R,4R)-1- (Methylsulfonyl)-4-((7- (3-morpholino-1H- pyrazol-4- yl)quinazolin-2- yl)amino)piperidin-3-ol | 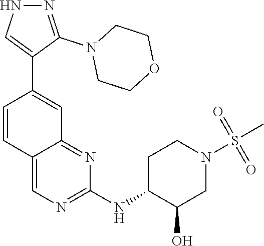 [SMILES: CS(=O)(=O)N1CC[C@@H](Nc2ncc3ccc(-c4c[nH]nc4N4CCOCC4)cc3n2)[C@H](O)C1] | LCMS found 474.2 |
| 56 | (3R,4R)-4-((7-(3- ((Dimethylamino) methyl)-1H-pyrazol-4- yl)quinazolin-2- yl)amino)-1- (methylsulfonyl) piperidin-3-ol | 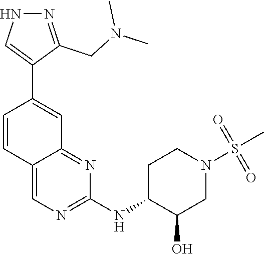 [SMILES: CN(C)Cc1n[nH]cc1-c1ccc2cnc(N[C@@H]3CCN(S(C)(=O)=O)C[C@H]3O)nc2c1] | LCMS found 446.2 |

TABLE 2
The compounds in Table 2 were prepared in accordance with the synthetic protocols set
forth in Example 28 using the appropriate starting materials.

| | | | Analytical |
|---|---|---|---|
| Ex. | Name | Structure | data |
| 57 | Methyl (3R,4R)-4-((7- (3- (Cyclopropylmethyl)- 1H-pyrazol-4- yl)quinazolin-2- yl)amino)-3- hydroxypiperidine-1- carboxylate | 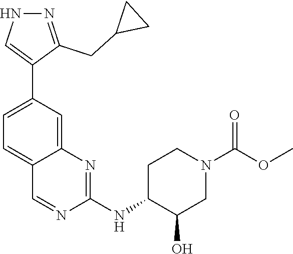 [SMILES: COC(=O)N1CC[C@@H](Nc2ncc3ccc(-c4c[nH]nc4CC4CC4)cc3n2)[C@H](O)C1] | LCMS found 423.2 |
| 58 | Methyl (3R,4R)-4-((7- (1-Cyclopropyl-1H- imidazol-5- yl)quinazolin-2- yl)amino)-3- hydroxypiperidine-1- carboxylate | 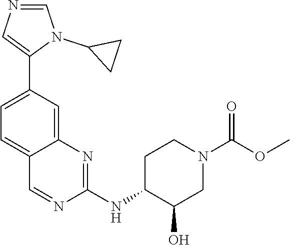 [SMILES: COC(=O)N1CC[C@@H](Nc2ncc3ccc(-c4cncn4C4CC4)cc3n2)[C@H](O)C1] | LCMS found 409.2 |
| 59 | Methyl (3R,4R)-4-((7- (1-ethyl-1H-imidazol- 5-yl)quinazolin-2- yl)amino)-3- hydroxypiperidine-1- carboxylate | 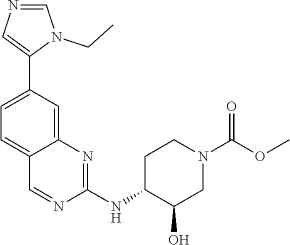 [SMILES: CCn1cncc1-c1ccc2cnc(N[C@@H]3CCN(C(=O)OC)C[C@H]3O)nc2c1] | LCMS found 397.2 |
| 60 | Methyl (3R,4R)-3-hydroxy-4- ((7-(3-morpholino-1H- pyrazol-4- yl)quinazolin-2- yl)amino)piperidine-1- carboxylate | 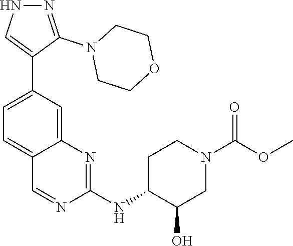 [SMILES: COC(=O)N1CC[C@@H](Nc2ncc3ccc(-c4c[nH]nc4N4CCOCC4)cc3n2)[C@H](O)C1] | LCMS found 454.2 |
| 61 | Methyl (3R,4R)-3- hydroxy-4-((7-(3- (tetrahydro-2H-pyran- 4-yl)-1H-pyrazol-4- yl)quinazolin-2- yl)amino)piperidine-1- carboxylate | 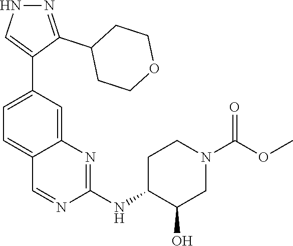 [SMILES: COC(=O)N1CC[C@@H](Nc2ncc3ccc(-c4c[nH]nc4C4CCOCC4)cc3n2)[C@H](O)C1] | LCMS found 453.2 |
| 62 | Methyl (3R,4R)-3- hydroxy-4-((7-(3-(2- hydroxyethyl)-1H- pyrazol-4- yl)quinazolin-2- yl)amino)piperidine-1- carboxylate | 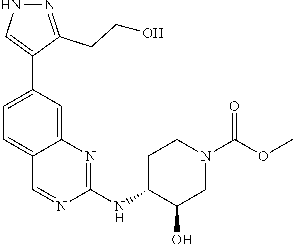 [SMILES: COC(=O)N1CC[C@@H](Nc2ncc3ccc(-c4c[nH]nc4CCO)cc3n2)[C@H](O)C1] | LCMS found 413.2 |
| 63 | Methyl (3R,4R)-4-((7- (3-(4-(azetidin-1- ylmethyl)-3- fluorophenyl)-1H- pyrazol-4- yl)quinazolin-2- yl)amino)-3- hydroxypiperidine-1- carboxylate | 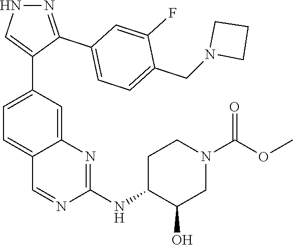 [SMILES: COC(=O)N1CC[C@@H](Nc2ncc3ccc(-c4c[nH]nc4-c4ccc(CN5CCC5)c(F)c4)cc3n2)[C@H](O)C1] | LCMS found 532.2 |
| 64 | Methyl (3R,4R)-4-((7- (3-(4- ((dimethylamino)methyl)- 3-fluorophenyl)-1H- pyrazol-4- yl)quinazolin-2- yl)amino)-3- hydroxypiperidine-1- carboxylate | 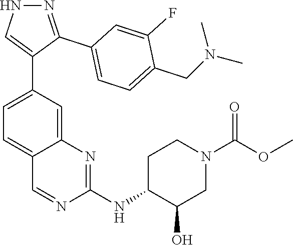 [SMILES: COC(=O)N1CC[C@@H](Nc2ncc3ccc(-c4c[nH]nc4-c4ccc(CN(C)C)c(F)c4)cc3n2)[C@H](O)C1] | LCMS found 520.2 |
| 65 | (3R,4R)-3-Hydroxy- N,N-dimethyl-4-((7-(3- methyl-1H-pyrazol-4- yl)quinazolin-2- yl)amino)piperidine-1- sulfonamide | 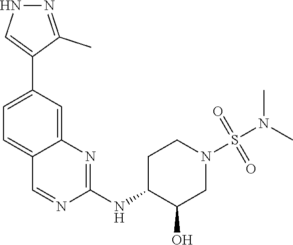 [SMILES: Cc1n[nH]cc1-c1ccc2cnc(N[C@@H]3CCN(S(=O)(=O)N(C)C)C[C@H]3O)nc2c1] | LCMS found 432.2 |

TABLE 3
The compounds in Table 3 were prepared in accordance with the synthetic protocols set
forth in Example 3 using the appropriate starting materials.

| | | | Analytical |
|---|---|---|---|
| Ex. | Name | Structure | data |
| 66 | (3R,4R)-4-((8-Fluoro- 7-(3-methyl-1H- pyrazol-4- yl)quinazolin-2- yl)amino)-1- (methylsulfonyl) piperidin-3-ol | 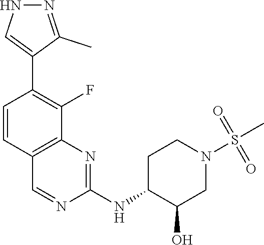 [SMILES: Cc1n[nH]cc1-c1ccc2cnc(N[C@@H]3CCN(S(C)(=O)=O)C[C@H]3O)nc2c1F] | LCMS found 421.1 |
| 67 | N-((3R,4R)-3-Methoxy- 1-(methylsulfonyl) piperidin- 4-yl)-7-(3-methyl- 1H-pyrazol-4- yl)quinazolin-2-amine | 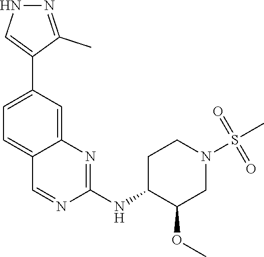 [SMILES: CO[C@@H]1CN(S(C)(=O)=O)CC[C@H]1Nc1ncc2ccc(-c3c[nH]nc3C)cc2n1] | LCMS found 417.2 |
| 68 | (3R,4R)-1- (Cyclopropylsulfonyl)- 4-((7-(3-ethyl-1H- pyrazol-4-yl)-6,8- difluoroquinazolin-2- yl)amino)piperidin-3-ol | 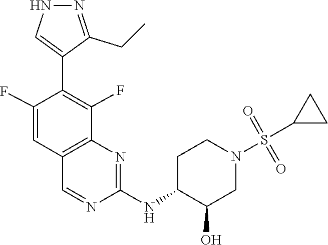 [SMILES: CCc1n[nH]cc1-c1c(F)cc2cnc(N[C@@H]3CCN(S(=O)(=O)C4CC4)C[C@H]3O)nc2c1F] | LCMS found 479.2 |
| 69 | (3R,4R)-4-((7-(3- Methyl-1H-pyrazol-4- yl)pyrido[2,3- d]pyrimidin-2- yl)amino)-1- (methylsulfonyl) piperidin-3-ol | 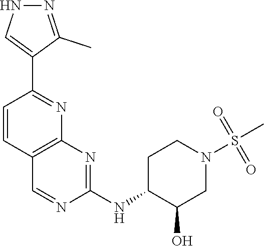 [SMILES: Cc1n[nH]cc1-c1ccc2cnc(N[C@@H]3CCN(S(C)(=O)=O)C[C@H]3O)nc2n1] | LCMS found 404.1 |
| 70 | (3R,4R)-4-((7-(1H- Pyrazol-4- yl)pyrido[4,3- d]pyrimidin-2- yl)amino)-1- (methylsulfonyl) piperidin-3-ol | 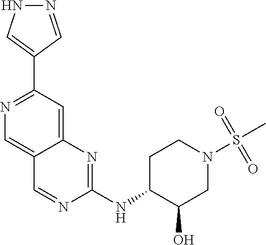 | LCMS found 404.1 |
| 71 | (3R,4R)-4-((7-(3- Methyl-1H-pyrazol-4- yl)pyrido[4,3- d]pyrimidin-2- yl)amino)-1- (methylsulfonyl) piperidin-3-ol | 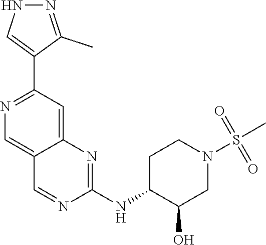 [SMILES: Cc1n[nH]cc1-c1cc2nc(N[C@@H]3CCN(S(C)(=O)=O)C[C@H]3O)ncc2cn1] | LCMS found 404.1 |
| 72 | (3R,4R)-4-((7-(3- Isopropyl-1H-pyrazol- 4-yl)pyrido[4,3- d]pyrimidin-2- yl)amino)-1- (methylsulfonyl) piperidin-3-ol | 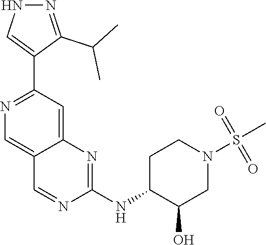 [SMILES: CC(C)c1n[nH]cc1-c1cc2nc(N[C@@H]3CCN(S(C)(=O)=O)C[C@H]3O)ncc2cn1] | LCMS found 432.2 |

### Example 73. (3R,4R)-4-((7-(1-(2,2-Difluoroethyl)-1H-imidazol-5-yl)quinazolin-2-yl)amino)-1-(methylsulfonyl)piperidin-3-ol

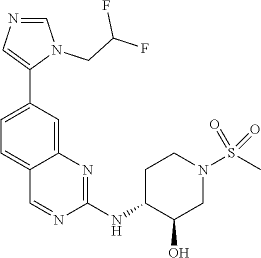 [SMILES: CS(=O)(=O)N1CC[C@@H](Nc2ncc3ccc(-c4cncn4CC(F)F)cc3n2)[C@H](O)C1]

To a mixture of (2-(((3R,4R)-3-hydroxy-1-(methylsulfonyl)piperidin-4-yl)amino)quinazolin-7-yl)boronic acid (Intermediate 21, 10.0 mg, 0.027 mmol) in 1,4-dioxane (0.5 mL) and H₂O (0.1 mL) was added 5-bromo-1-(2,2-difluoroethyl)-1H-imidazole (8.6 mg, 0.041 mmol), XPhos Pd G4 (2.4 mg, 2.73 μmol), and sodium carbonate (4.3 mg, 0.04 mmol). The reaction mixture was stirred at 90° C. for 1 h. After cooling to r.t., the reaction mixture was diluted with MeOH, filtered and purified by prep-HPLC (Sunfire C18 column, eluting with a gradient of acetonitrile/water containing 0.1% NH₄OH, at flow rate of 60 mL/min) to afford the desired product. LC-MS calculated for C₁₉H₂₃F₂N₆O₃S (M+H)⁺: m/z=453.2; found 453.2.

### Example 74. (3R,4R)-4-((7-(3-(1-Methyl-1,2,3,6-tetrahydropyridin-4-yl)-1H-pyrazol-4-yl)quinazolin-2-yl)amino)-1-(methylsulfonyl)piperidin-3-ol

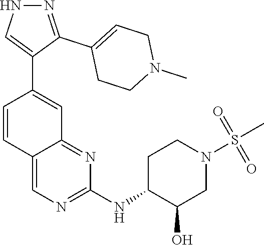 [SMILES: CN1CC=C(c2n[nH]cc2-c2ccc3cnc(N[C@@H]4CCN(S(C)(=O)=O)C[C@H]4O)nc3c2)CC1]

To a mixture of (3R,4R)-4-((7-(3-(1-methyl-1,2,3,6-tetrahydropyridin-4-yl)-1-(tetrahydro-2H-pyran-2-yl)-1H-pyrazol-4-yl)quinazolin-2-yl)amino)-1-(methylsulfonyl)piperidin-3-ol (Intermediate 24, 5 mg, 8.8 μmol) in MeOH (0.25 mL) was added TFA (0.25 mL, 3.24 mmol). The reaction mixture was stirred at r.t. for 1 h. The resulting mixture was filtered and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₃H₃₀N₇O₃S (M+H)⁺: m/z=484.2; found 484.3.

### Example 75. (3R,4R)-4-((7-(3-(1-Methylpiperidin-4-yl)-1H-pyrazol-4-yl)quinazolin-2-yl)amino)-1-(methylsulfonyl)piperidin-3-ol

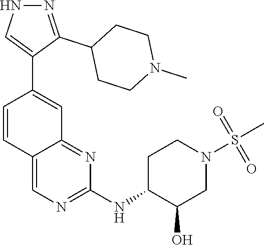 [SMILES: CN1CCC(c2n[nH]cc2-c2ccc3cnc(N[C@@H]4CCN(S(C)(=O)=O)C[C@H]4O)nc3c2)CC1]

To a mixture of (3R,4R)-4-((7-(3-(1-methyl-1,2,3,6-tetrahydropyridin-4-yl)-1-(tetrahydro-2H-pyran-2-yl)-1H-pyrazol-4-yl)quinazolin-2-yl)amino)-1-(methylsulfonyl)piperidin-3-ol (Intermediate 24, 5 mg, 8.8 μmol) in MeOH (0.5 mL) was added Pd—C (10 wt. %, 5.0 mg, 0.047 mmol) and the reaction mixture was equipped with a hydrogen balloon. The mixture was stirred at r.t. for 1 h. TFA (0.25 mL, 3.24 mmol) was added to the reaction mixture and the mixture was stirred at r.t. for 1 h. The resulting mixture was filtered and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₃H₃₂N₇O₃S (M+H)⁺: m/z=486.2; found 486.3.

### Example 76. (3R,4R)-4-((7-(3-(2-Methoxypyridin-4-yl)-1H-pyrazol-4-yl)quinazolin-2-yl)amino)-1-(methylsulfonyl)piperidin-3-ol

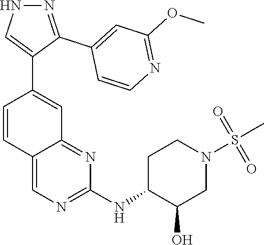 [SMILES: COc1cc(-c2n[nH]cc2-c2ccc3cnc(N[C@@H]4CCN(S(C)(=O)=O)C[C@H]4O)nc3c2)ccn1]

To a mixture of (3R,4R)-4-((7-(3-bromo-1-(tetrahydro-2H-pyran-2-yl)-1H-pyrazol-4-yl)quinazolin-2-yl)amino)-1-(methylsulfonyl)piperidin-3-ol (Intermediate 23, 15.0 mg, 0.027 mmol) in 1,4-dioxane (0.5 mL) and H₂O (0.1 mL) was added (2-methoxypyridin-4-yl)boronic acid (6.2 mg, 0.041 mmol), XPhos Pd G4 (2.3 mg, 2.72 μmol), and sodium carbonate (4.3 mg, 0.041 mmol). The reaction mixture was stirred at 90° C. for 1 h. After cooling to r.t., TFA (0.25 mL, 3.24 mmol) was added to the reaction mixture and the mixture was stirred at r.t. for 1 h. The resulting mixture was filtered and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₃H₂₆N₇O₄S (M+H)⁺: m/z=496.2; found 496.2.

### Example 77. (3R,4R)-4-((7-(5-(3-Methoxyprop-1-yn-1-yl)-1H-pyrazol-4-yl)quinazolin-2-yl)amino)-1-(methylsulfonyl)piperidin-3-ol

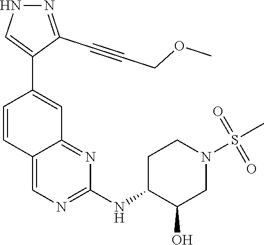 [SMILES: COCC#Cc1n[nH]cc1-c1ccc2cnc(N[C@@H]3CCN(S(C)(=O)=O)C[C@H]3O)nc2c1]

### Step 1. 4-Bromo-3-(3-methoxyprop-1-yn-1-yl)-1-(tetrahydro-2H-pyran-2-yl)-1H-pyrazole

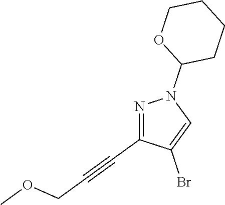 [SMILES: COCC#Cc1nn(C2CCCCO2)cc1Br]

To a mixture of 4-bromo-3-iodo-1-(tetrahydro-2H-pyran-2-yl)-1H-pyrazole (Intermediate 25, 40 mg, 0.11 mmol) in THF (0.5 mL) was added 3-methoxyprop-1-yne (0.014 mL, 0.17 mmol), bis(triphenylphosphine)palladium(II) dichloride (7.9 mg, 0.011 mmol), copper(I) iodide (2.1 mg, 0.011 mmol) and triethylamine (0.047 mL, 0.34 mmol). The mixture was sparged with N₂ for 5 min and stirred at 90° C. for 2 h. The reaction mixture was cooled to r.t., diluted with water and extracted with EtOAc. The combined organic layers were washed with water and brine, dried over MgSO₄, and concentrated. The crude residue was purified by flash column chromatography (SiO₂, EtOAc/hexanes) to afford the desired product. LC-MS calculated for C₇H₈BrN₂O (M+H-C₅H₈O)⁺: m/z=215.0; found 214.8.

### Step 2. (3R,4R)-4-((7-(5-(3-Methoxyprop-1-yn-1-yl)-1H-pyrazol-4-yl)quinazolin-2-yl)amino)-1-(methylsulfonyl)piperidin-3-ol

To a mixture of (2-(((3R,4R)-3-hydroxy-1-(methylsulfonyl)piperidin-4-yl)amino)quinazolin-7-yl)boronic acid (Intermediate 21, 10.0 mg, 0.027 mmol) in 1,4-dioxane (0.5 mL) and H₂O (0.1 mL) was added 4-bromo-3-(3-methoxyprop-1-yn-1-yl)-1-(tetrahydro-2H-pyran-2-yl)-1H-pyrazole (Step 1, 9.8 mg, 0.033 mmol), XPhos Pd G4 (2.4 mg, 2.7 μmol), and sodium carbonate (4.3 mg, 0.04 mmol). The reaction mixture was stirred at 90° C. for 1 h. After cooling to r.t., TFA (0.25 mL, 3.24 mmol) was added to the reaction mixture and the mixture was stirred at r.t. for 1 h. The resulting mixture was filtered and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₁H₂₅N₆O₄S (M+H)⁺: m/z=457.2; found 457.1.

### Example 78. (3R,4R)-1-(Cyclopropylsulfonyl)-4-((6,8-difluoro-7-(3-methyl-1H-pyrazol-4-yl)quinazolin-2-yl)amino)piperidin-3-ol

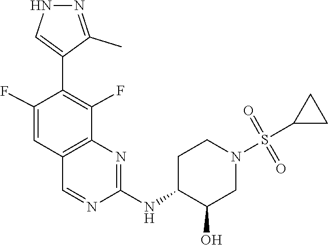 [SMILES: Cc1n[nH]cc1-c1c(F)cc2cnc(N[C@@H]3CCN(S(=O)(=O)C4CC4)C[C@H]3O)nc2c1F]

The title compound was prepared according to the procedures outlined for Example 1, with tert-butyl 3-methyl-4-(4,4,5,5-tetramethyl-1,3,2-dioxaborolan-2-yl)-1H-pyrazole-1-carboxylate) replacing 1-(1-ethoxyethyl)-4-(4,4,5,5-tetramethyl-1,3,2-dioxaborolan-2-yl)-1H-pyrazole and (3R,4R)-4-((7-bromo-6,8-difluoroquinazolin-2-yl)amino)-1-(cyclopropylsulfonyl)piperidin-3-ol (Intermediate 27) replacing (3R,4R)-4-((7-bromoquinazolin-2-yl)amino)-1-(methylsulfonyl)piperidin-3-ol (Intermediate 3) in Step 1. LC-MS calculated for C₂₀H₂₃F₂N₆O₃S (M+H)⁺: m/z=465.2; found 465.1. ¹H NMR (500 MHz, DMSO-d₆) δ 9.15 (s, 1H), 7.74 (s, 1H), 7.62-7.56 (m, 2H), 3.94 (s, 1H), 3.70-3.61 (m, 2H), 3.56 (dd, J=11.0, 5.9 Hz, 1H), 3.03-2.94 (m, 1H), 2.79 (t, J=10.8 Hz, 1H), 2.67-2.58 (m, 1H), 2.20 (s, 3H), 2.13-2.05 (m, 1H), 1.62-1.50 (m, 1H), 1.06-0.89 (m, 4H).

TABLE 4
The compounds in Table 4 were prepared in accordance with the synthetic protocols set
forth in Example 78 using the appropriate starting materials.

| | | | Analytical |
|---|---|---|---|
| Ex. | Name | Structure | data |
| 79 | (3R,4R)-4-((7-(3-Ethyl- 1H-pyrazol-4-yl)-6,8- difluoroquinazolin-2- yl)amino)-1- (methylsulfonyl) piperidin-3-ol | 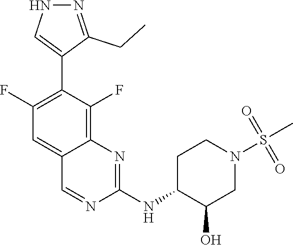 [SMILES: CCc1n[nH]cc1-c1c(F)cc2cnc(N[C@@H]3CCN(S(C)(=O)=O)C[C@H]3O)nc2c1F] | LCMS found 453.1 |
| 80 | (3R,4R)-4-((6,8- Difluoro-7-(3-methyl- 1H-pyrazol-4- yl)quinazolin-2- yl)amino)-1- (methylsulfonyl) piperidin-3-ol | 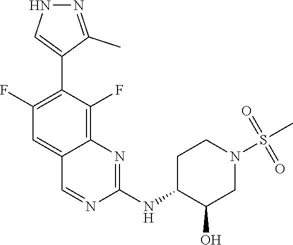 [SMILES: Cc1n[nH]cc1-c1c(F)cc2cnc(N[C@@H]3CCN(S(C)(=O)=O)C[C@H]3O)nc2c1F] | LCMS found 439.1 |
| 81 | (3R,4R)-4-((6,8- Difluoro-7-(1-methyl- 1H-imidazol-5- yl)quinazolin-2- yl)amino)-1- (methylsulfonyl) piperidin-3-ol | 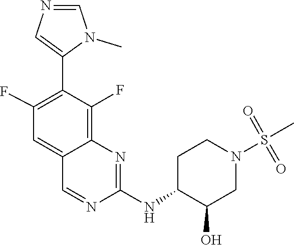 [SMILES: Cn1cncc1-c1c(F)cc2cnc(N[C@@H]3CCN(S(C)(=O)=O)C[C@H]3O)nc2c1F] | LCMS found 439.1 |

### Example 82. (3R,4R)-4-((6-Fluoro-7-(3-methyl-1H-pyrazol-4-yl)quinazolin-2-yl)amino)-1-(methylsulfonyl)piperidin-3-ol

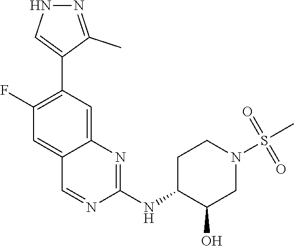 [SMILES: Cc1n[nH]cc1-c1cc2nc(N[C@@H]3CCN(S(C)(=O)=O)C[C@H]3O)ncc2cc1F]

The title compound was prepared according to the procedures outlined for Example 1, with (3R,4R)-4-((7-chloro-6-fluoroquinazolin-2-yl)amino)-1-(cyclopropylsulfonyl)piperidin-3-ol (Intermediate 29) replacing (3R,4R)-4-((7-chloro-6-fluoroquinazolin-2-yl)amino)-1-(methylsulfonyl)piperidin-3-ol (Intermediate 28). LC-MS calculated for C₁₈H₂₂FN₆O₃S (M+H)⁺: m/z=421.1; found 421.2

TABLE 5
The compounds in Table 5 were prepared in accordance with the synthetic protocols set
forth in Example 82 using the appropriate starting materials.

| | | | Analytical |
|---|---|---|---|
| Ex. | Name | Structure | data |
| 83 | (3R,4R)-4-((6-Fluoro- 7-(1H-pyrazol-4- yl)quinazolin-2- yl)amino)-1- (methylsulfonyl) piperidin-3-ol | 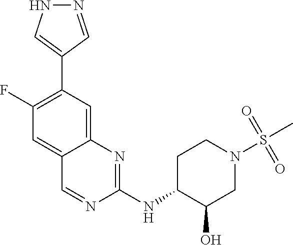 [SMILES: CS(=O)(=O)N1CC[C@@H](Nc2ncc3cc(F)c(-c4cn[nH]c4)cc3n2)[C@H](O)C1] | LCMS found 407.2 |
| 84 | (3R,4R)-4-((7-(3- Cyclopropyl-1H- pyrazol-4-yl)-6- fluoroquinazolin-2- yl)amino)-1- (methylsulfonyl) piperidin-3-ol | 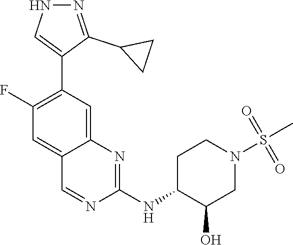 [SMILES: CS(=O)(=O)N1CC[C@@H](Nc2ncc3cc(F)c(-c4c[nH]nc4C4CC4)cc3n2)[C@H](O)C1] | LCMS found 447.2 |
| 85 | (3R,4R)-4-((7-(3- (Difluoromethyl)-1H- pyrazol-4-yl)-6- fluoroquinazolin-2- yl)amino)-1- (methylsulfonyl) piperidin-3-ol | 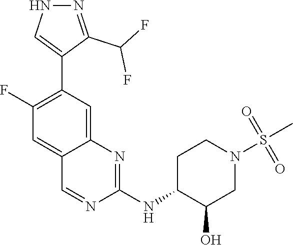 [SMILES: CS(=O)(=O)N1CC[C@@H](Nc2ncc3cc(F)c(-c4c[nH]nc4C(F)F)cc3n2)[C@H](O)C1] | LCMS found 457.1 |
| 86 | (3R,4R)-4-((6-Fluoro- 7-(3-fluoro-1H- pyrazol-4- yl)quinazolin-2- yl)amino)-1- (methylsulfonyl) piperidin-3-ol | 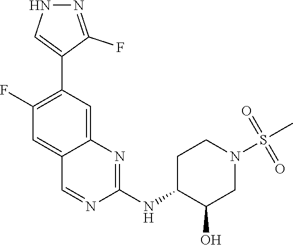 [SMILES: CS(=O)(=O)N1CC[C@@H](Nc2ncc3cc(F)c(-c4c[nH]nc4F)cc3n2)[C@H](O)C1] | LCMS found 407.1 |
| 87 | (3R,4R)-4-((6-Fluoro- 7-(1-methyl-1H- imidazol-5- yl)quinazolin-2- yl)amino)-1- (methylsulfonyl) piperidin-3-ol | 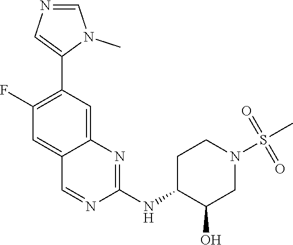 [SMILES: Cn1cncc1-c1cc2nc(N[C@@H]3CCN(S(C)(=O)=O)C[C@H]3O)ncc2cc1F] | LCMS found 421.1 |

### Example 88. (3R,4R)-1-(Cyclopropylsulfonyl)-4-((6-fluoro-7-(1-methyl-1H-imidazol-5-yl)quinazolin-2-yl)amino)piperidin-3-ol

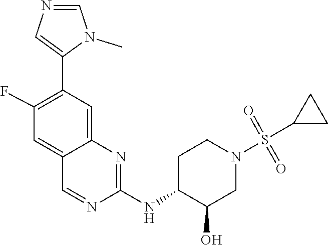 [SMILES: Cn1cncc1-c1cc2nc(N[C@@H]3CCN(S(=O)(=O)C4CC4)C[C@H]3O)ncc2cc1F]

The title compound was prepared according to the procedures outlined for Example 1, with 1-methyl-5-(4,4,5,5-tetramethyl-1,3,2-dioxaborolan-2-yl)-1H-imidazole replacing 1-(1-ethoxyethyl)-4-(4,4,5,5-tetramethyl-1,3,2-dioxaborolan-2-yl)-1H-pyrazole and (3R,4R)-4-((7-chloro-6-fluoroquinazolin-2-yl)amino)-1-(cyclopropylsulfonyl)piperidin-3-ol replacing (3R,4R)-4-((7-bromoquinazolin-2-yl)amino)-1-(methylsulfonyl)piperidin-3-ol. LC-MS calculated for C₁₈H₂₂FN₆O₃S (M+H)⁺: m/z=421.1; found 421.2.

### Example 89. (3R,4R)-4-((6-Fluoro-7-(1-methyl-1H-imidazol-5-yl)quinazolin-2-yl)amino)-3-hydroxypiperidin-1-yl)(1-(trifluoromethyl)cyclopropyl)methanone

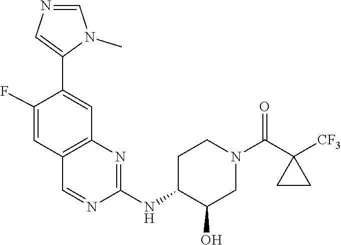 [SMILES: Cn1cncc1-c1cc2nc(N[C@@H]3CCN(C(=O)C4(C(F)(F)F)CC4)C[C@H]3O)ncc2cc1F]

A mixture of (3R,4R)-4-((6-fluoro-7-(1-methyl-1H-imidazol-5-yl)quinazolin-2-yl)amino)piperidin-3-ol hydrochloride (Intermediate 31, 20.0 mg, 0.053 mmol), 2-(3H-[1,2,3]triazolo[4,5-b]pyridin-3-yl)-1,1,3,3-tetramethylisouronium hexafluorophosphate(V) (30.1 mg, 0.079 mmol), N-ethyl-N-isopropylpropan-2-amine (27.3 mg, 0.211 mmol), 1-(trifluoromethyl)cyclopropane-1-carboxylic acid (12.2 mg, 0.079 mmol), and CH₃CN (1.1 mL) was stirred at 65° C. for 0.5 h. The reaction mixture was diluted with CH₃CN, filtered, and purified with prep-HPLC (Sunfire C18 column, eluting with a gradient of acetonitrile/water containing 0.1% NH₄OH, at flow rate of 60 mL/min) to afford the desired product. LC-MS calculated for C₂₂H₂₃F₄N₆O₂(M+H)⁺: m/z=479.2; found 479.2.

TABLE 6
The compounds in Table 6 were prepared in accordance with the synthetic protocols set
forth in Example 89 using the appropriate starting materials.

| | | | Analytical |
|---|---|---|---|
| Ex. | Name | Structure | data |
| 90 | (1-(Difluoromethyl) cyclopropyl) ((3R,4R)-4-((6- fluoro-7-(1-methyl-1H- imidazol-5- yl)quinazolin-2- yl)amino)-3- hydroxypiperidin-1- yl)methanone | 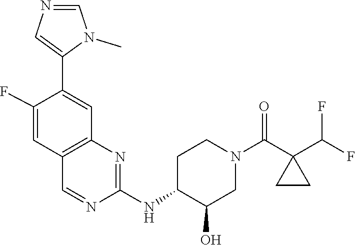 [SMILES: Cn1cncc1-c1cc2nc(N[C@@H]3CCN(C(=O)C4(C(F)F)CC4)C[C@H]3O)ncc2cc1F] | LCMS found 461.2 |
| 91 | ((3R,4R)-4-((6-Fluoro- 7-(1-methyl-1H- imidazol-5- yl)quinazolin-2- yl)amino)-3- hydroxypiperidin-1- yl)(1- methylcyclopropyl) methanone | 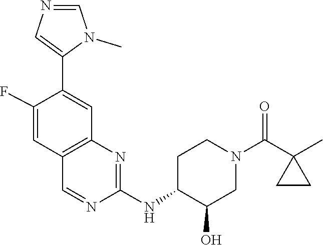 [SMILES: Cn1cncc1-c1cc2nc(N[C@@H]3CCN(C(=O)C4(C)CC4)C[C@H]3O)ncc2cc1F] | LCMS found 425.2 |

### Example 92. (3R,4R)-1-(Cyclopropylsulfonyl)-4-((6-fluoro-7-(1-methyl-1H-pyrazol-5-yl)quinazolin-2-yl)amino)piperidin-3-ol

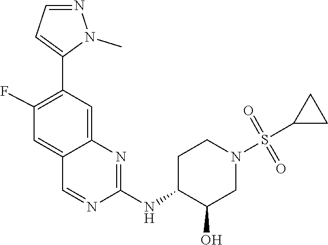 [SMILES: Cn1nccc1-c1cc2nc(N[C@@H]3CCN(S(=O)(=O)C4CC4)C[C@H]3O)ncc2cc1F]

The title compound was prepared according to the procedures outlined for Example 88, with 1-methyl-5-(4,4,5,5-tetramethyl-1,3,2-dioxaborolan-2-yl)-1H-imidazole replacing 1-methyl-5-(4,4,5,5-tetramethyl-1,3,2-dioxaborolan-2-yl)-1H-pyrazole. LC-MS calculated for C₂₀H₂₄FN₆O₃S (M+H)⁺: m/z=447.2; found 447.2.

### Example 93. (3R,4R)-4-((5-Fluoro-7-(1-methyl-1H-imidazol-5-yl)quinazolin-2-yl)amino)-1-(methylsulfonyl)piperidin-3-ol

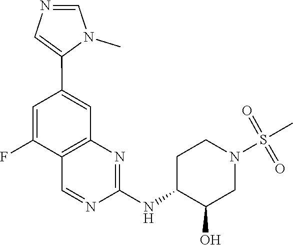 [SMILES: Cn1cncc1-c1cc(F)c2cnc(N[C@@H]3CCN(S(C)(=O)=O)C[C@H]3O)nc2c1]

The title compound was prepared according to the procedures outlined for Example 1, with (3R,4R)-4-((7-bromo-5-fluoroquinazolin-2-yl)amino)-1-(methylsulfonyl)piperidin-3-ol (Intermediate 32) replacing (3R,4R)-4-((7-bromoquinazolin-2-yl)amino)-1-(methylsulfonyl)piperidin-3-ol (Intermediate 3). LC-MS calculated for C₁₈H₂₂FN₆O₃S (M+H)⁺: m/z=421.1; found 421.2.

TABLE 7
The compounds in Table 7 were prepared in accordance with the synthetic protocols set
forth in Example 93 using the appropriate starting materials.

| | | | Analytical |
|---|---|---|---|
| Ex. | Name | Structure | data |
| 94 | (3R,4R)-4-((5-Fluoro- 7-(3-methyl-1H- pyrazol-4- yl)quinazolin-2- yl)amino)-1- (methylsulfonyl) piperidin-3-ol | 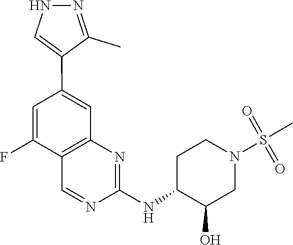 [SMILES: Cc1n[nH]cc1-c1cc(F)c2cnc(N[C@@H]3CCN(S(C)(=O)=O)C[C@H]3O)nc2c1] | LCMS found 421.1 |
| 95 | (3R,4R)-4-((5-Fluoro- 7-(1H-pyrazol-4- yl)quinazolin-2- yl)amino)-1- (methylsulfonyl) piperidin-3-ol | 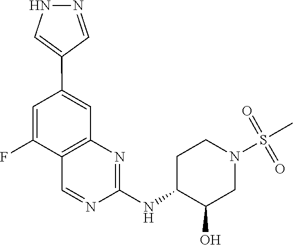 [SMILES: CS(=O)(=O)N1CC[C@@H](Nc2ncc3c(F)cc(-c4cn[nH]c4)cc3n2)[C@H](O)C1] | LCMS found 407.1 |

TABLE 8
The compounds in Table 8 were prepared in accordance with the synthetic protocols set
forth in Example 82 using the appropriate starting materials.

| | | | Analytical |
|---|---|---|---|
| Ex. | Name | Structure | data |
| 96 | (3R,4R)-4-((6-Fluoro- 7-(1-(2- (methylamino)ethyl)- 1H-pyrazol-4- yl)quinazolin-2- yl)amino)-1- (methylsulfonyl) piperidin-3-ol | 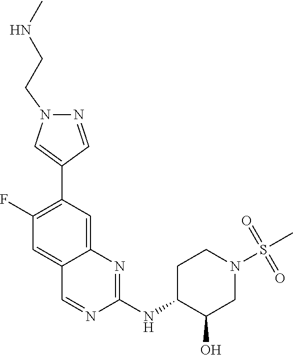 [SMILES: CNCCn1cc(-c2cc3nc(N[C@@H]4CCN(S(C)(=O)=O)C[C@H]4O)ncc3cc2F)cn1] | LCMS found 464.1 |
| 97 | (3R,4R)-4-((6,8- Difluoro-7-(1-(2- (methylamino)ethyl)- 1H-pyrazol-4- yl)quinazolin-2- yl)amino)-1- (methylsulfonyl) piperidin-3-ol | 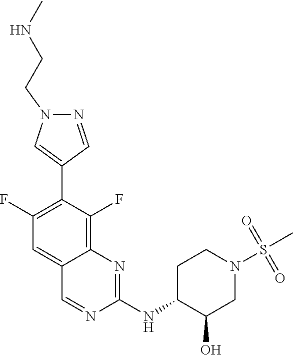 [SMILES: CNCCn1cc(-c2c(F)cc3cnc(N[C@@H]4CCN(S(C)(=O)=O)C[C@H]4O)nc3c2F)cn1] | LCMS found 482.2 |
| 98 | (3R,4R)-4-((7-(1-(2- (Methylamino)ethyl)- 1H-pyrazol-4- yl)quinazolin-2- yl)amino)-1- (methylsulfonyl) piperidin-3-ol | 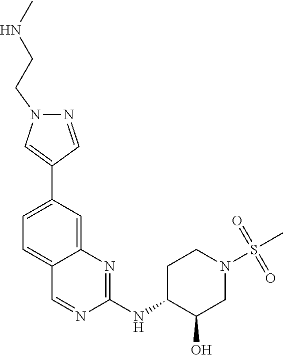 [SMILES: CNCCn1cc(-c2ccc3cnc(N[C@@H]4CCN(S(C)(=O)=O)C[C@H]4O)nc3c2)cn1] | LCMS found 446.2 |

TABLE 9
The compounds in Table 9 were prepared in accordance with the synthetic protocols set
forth in Example 89 using the appropriate starting materials.

| | | | Analytical |
|---|---|---|---|
| Ex. | Name | Structure | data |
| 99 | ((3R,4R)-4-((6-Fluoro- 7-(1-(2- (methylamino)ethyl)- 1H-pyrazol-4- yl)quinazolin-2- yl)amino)-3- hydroxypiperidin-1- yl)(1- methylcyclopropyl) methanone | 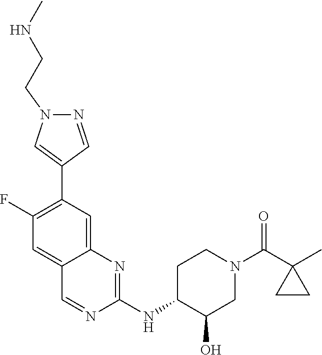 [SMILES: CNCCn1cc(-c2cc3nc(N[C@@H]4CCN(C(=O)C5(C)CC5)C[C@H]4O)ncc3cc2F)cn1] | LCMS found 468.2 |
| 100 | (3,3- Difluorocyclobutyl) ((3R,4R)-4-((6-fluoro-7- (1-(2- (methylamino)ethyl)- 1H-pyrazol-4- yl)quinazolin-2- yl)amino)-3- hydroxypiperidin-1- yl)methanone | 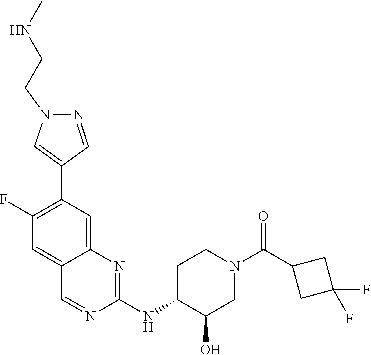 [SMILES: CNCCn1cc(-c2cc3nc(N[C@@H]4CCN(C(=O)C5CC(F)(F)C5)C[C@H]4O)ncc3cc2F)cn1] | LCMS found 504.2 |
| 101 | 2,2-Difluoroethyl (3R,4R)-4-((6-fluoro-7- (1-(2- (methylamino)ethyl)- 1H-pyrazol-4- yl)quinazolin-2- yl)amino)-3- hydroxypiperidine-1- carboxylate | 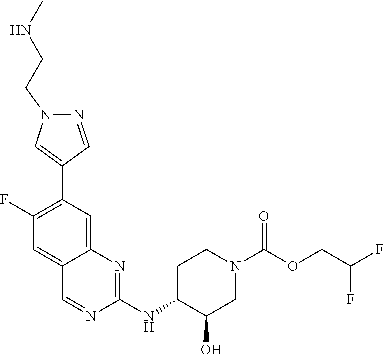 [SMILES: CNCCn1cc(-c2cc3nc(N[C@@H]4CCN(C(=O)OCC(F)F)C[C@H]4O)ncc3cc2F)cn1] | LCMS found 494.2 |
| 102 | ((3R,4R)-4-((6-Fluoro- 7-(1-(2- (methylamino)ethyl)- 1H-pyrazol-4- yl)quinazolin-2- yl)amino)-3- hydroxypiperidin-1- yl)(1- (trifluoromethyl) cyclobutyl)methanone | 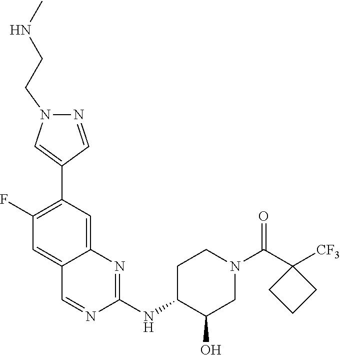 [SMILES: CNCCn1cc(-c2cc3nc(N[C@@H]4CCN(C(=O)C5(C(F)(F)F)CCC5)C[C@H]4O)ncc3cc2F)cn1] | LCMS found 536.2 |
| 103 | (1-(Difluoromethyl) cyclopropyl) ((3R,4R)-4-((6- fluoro-7-(1-(2- (methylamino)ethyl)- 1H-pyrazol-4- yl)quinazolin-2- yl)amino)-3- hydroxypiperidin-1- yl)methanone | 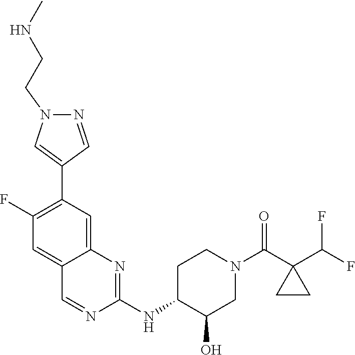 [SMILES: CNCCn1cc(-c2cc3nc(N[C@@H]4CCN(C(=O)C5(C(F)F)CC5)C[C@H]4O)ncc3cc2F)cn1] | LCMS found 504.2 |

TABLE 9
The compounds in Table 9 were prepared in accordance with the synthetic protocols set
forth in Example 12 using the appropriate starting materials.

| | | | Analytical |
|---|---|---|---|
| Ex. | Name | Structure | data |
| 104 | Methyl (3R,4R)-4-((6- fluoro-7-(1H-pyrazol- 4-yl)quinazolin-2- yl)amino)-3- hydroxypiperidine-1- carboxylate | 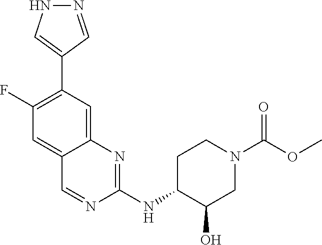 [SMILES: COC(=O)N1CC[C@@H](Nc2ncc3cc(F)c(-c4cn[nH]c4)cc3n2)[C@H](O)C1] | LCMS found 387.2 |
| 105 | Methyl (3R,4R)-4-((7- (3-ethyl-1H-pyrazol-4- yl)-6-fluoroquinazolin- 2-yl)amino)-3- hydroxypiperidine-1- carboxylate | 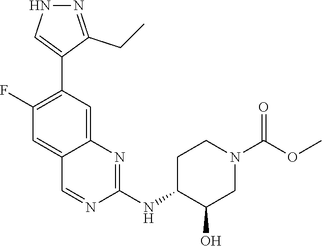 [SMILES: CCc1n[nH]cc1-c1cc2nc(N[C@@H]3CCN(C(=O)OC)C[C@H]3O)ncc2cc1F] | LCMS found 415.2 |
| 106 | Methyl (3R,4R)-4-((6- fluoro-7-(3-methyl-1H- pyrazol-4- yl)quinazolin-2- yl)amino)-3- hydroxypiperidine-1- carboxylate | 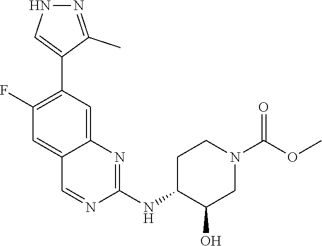 [SMILES: COC(=O)N1CC[C@@H](Nc2ncc3cc(F)c(-c4c[nH]nc4C)cc3n2)[C@H](O)C1] | LCMS found 401.2 |

### Example A. CDK HTRF Enzyme Activity Assay

CDK/Cyclin enzyme activity assays utilize N-terminally tagged (GST, HIS, or Flag) full-length human CDK and Cyclin proteins co-expressed in a baculovirus expression system (CDK4/Cyclin D1, ProQinase 0142-0143-1; CDK6/Cyclin D3, ProQinase 0051-0373-1; CDK2/Cyclin E1, Carna Biosciences 04-165-500; CDK1/Cyclin B1, Carna Biosciences 04-102-100; CDK9/Cyclin T1, Cama Biosciences 04-110-100; CDK7/Cyclin H/MAT1, Millipore-Sigma 14-476).

Assays were conducted in white 384-well polystyrene plates in a final reaction volume of 8 μL. CDK/Cyclin was pre-incubated with the compounds of the Examples (10 or 40 nL serially diluted in DMSO) in 4 μL assay buffer (containing 50 mM HEPES pH 7.5, I mM EGTA, 10 mM MgCl, 2 mM DTT, 0.05 mg/mL BSA, and 0.01% Tween 20) for 30 minutes at room temperature, after which reactions were started by the addition of 4 μL ATP and U-light peptide ((eIF4E-Binding Protein, Revvity 0128, or Myelin Basic Protein, Revvity 0109), for a final concentration of 1 mM and 50 nM, respectively. The reactions were stopped after 60 minutes by the addition of EDTA and Europium-labeled anti-phospho-peptide antibody, for a final concentration of 15 mM and 1 nM, respectively. TR-FRET signals were read after 15 minutes at room temperature on a PheraSTAR FSX plate reader (BMG Labtech). Data was analyzed with Genedata Screener software using a three or four parameter dose response curve to determine IC₅₀ for each compound.

Compounds of the present disclosure, as exemplified in Examples 1-26, showed IC₅₀ values in the following ranges: +=IC₅₀≤10 nM; ++=10 nM<IC₅₀≤100 nM; +++=100 nM<IC₅₀≤1000 nM; ++++=IC₅₀>1000 nM;

Data obtained for the Example compounds using the CDK2/Cyclin E1 and CDK4/Cyclin D1 homogenous time-resolved fluorescence (HTRF) enzyme activity assays described in Example A is provided in Table A.

TABLE A

| Example | CDK2/Cyclin E1 IC₅₀ (nM) | CDK4/Cyclin D1 IC₅₀ (nM) |
|---|---|---|
| 1 | ++ | + |
| 2 | +++ | + |
| 3 | + | + |
| 4 | +++ | + |
| 5 | + | + |
| 6 | ++++ | + |
| 7 | ++ | + |
| 8 | + | + |
| 9 | + | + |
| 10 | + | + |
| 11 | + | + |
| 12 | +++ | + |
| 13 | ++ | + |
| 14 | +++ | + |
| 15 | ++ | + |
| 16 | ++ | + |
| 17 | ++ | + |
| 18 | ++ | + |
| 19 | ++ | + |
| 20 | ++ | + |
| 21 | ++ | + |
| 22 | ++ | + |
| 23 | + | + |
| 24 | + | + |
| 25 | + | + |
| 26 | +++ | + |
| 27 | + | + |
| 28 | ++ | + |
| 29 | ++ | + |
| 30 | ++ | + |
| 31 | +++ | + |
| 32 | ++++ | ++ |
| 33 | +++ | ++ |
| 34 | +++ | ++ |
| 35 | + | + |
| 36 | ++ | + |
| 37 | +++ | + |
| 38 | ++++ | ++ |
| 39 | + | + |
| 40 | ++++ | ++ |
| 41 | +++ | ++ |
| 42 | ++ | + |
| 43 | +++ | + |
| 44 | +++ | + |
| 45 | + | + |
| 46 | ++++ | + |
| 47 | ++ | + |
| 48 | +++ | + |
| 49 | +++ | + |
| 50 | + | + |
| 51 | + | + |
| 52 | ++ | + |
| 53 | ++ | + |
| 54 | + | + |
| 55 | + | + |
| 56 | + | + |
| 57 | +++ | + |
| 58 | ++ | + |
| 59 | ++ | + |
| 60 | +++ | + |
| 61 | +++ | + |
| 62 | +++ | + |
| 63 | +++ | + |
| 64 | +++ | + |
| 65 | + | + |
| 66 | ++ | + |
| 67 | + | + |
| 68 | + | + |
| 69 | ++ | + |
| 70 | +++ | + |
| 71 | ++ | + |
| 72 | + | + |
| 73 | + | + |
| 74 | + | + |
| 75 | + | + |
| 76 | ++ | + |
| 77 | +++ | + |
| 78 | + | + |
| 79 | + | + |
| 80 | + | + |
| 81 | ++ | + |
| 82 | + | + |
| 83 | ++ | + |
| 84 | + | + |
| 85 | ++ | + |
| 86 | ++ | + |
| 87 | + | + |
| 88 | + | + |
| 89 | +++ | + |
| 90 | +++ | + |
| 91 | ++++ | + |
| 92 | + | + |
| 93 | + | + |
| 94 | ++ | + |
| 95 | ++ | + |
| 96 | ++ | + |
| 97 | ++ | + |
| 98 | + | + |
| 99 | ++++ | + |
| 100 | ++++ | + |
| 101 | ++++ | + |
| 102 | ++++ | + |
| 103 | ++++ | + |
| 104 | +++ | + |
| 105 | ++ | + |
| 106 | +++ | + |

### Example B. CDK2 pRb S780 HTRF Assay in COV318 Cells

COV318 cells (Sigma-Aldrich, cat #07071903) are cultured in 5% FBS DMEM and plated in a 96-well flat-bottom plate (Corning-Costar, cat #3596) at 25,000 cells per well in 100 μL volume. At a starting concentration of 5 mM (final assay concentration 20 μM), compounds are 3-fold serially diluted in DMSO. 249 μL of 5% FBS DMEM media is added to 1 μL of compound, media is aspirated from cells, and 100 μL of diluted compound are added to the cell plate. The plates are then incubated overnight for 22 hours at 37° C. and 5% CO₂. The next day, the plates are washed to remove supernatant and 50 μL of 4× Revvity lysis buffer #2 from the phospho-Rb (Ser780) cellular assay kit (Revvity Rb phosphor-S780 kit, cat #64RBS780PEH) is diluted to 1× in distilled water and supplemented with blocking buffer (Revvity Rb phosphor-S780 kit, cat #64RBS780PEH) and protease inhibitor cocktail set III, EDTA-free (Calbiochem, cat #539134) and added to each well. The plate is then gently shaken at room temperature for 45 minutes to 1 hour until cell lysis was completed. Then, 4 μL of a 1:1 mixture of diluted donor fluorophore solution and diluted acceptor fluorophore solution (Revvity Rb phosphor-S780 kit, cat #64RBS780PEH) is added into a 384-well white plate (Greiner, cat #784075). 16 μL of cell lysate is transferred into each well for a final volume of 20 μL per well. After a 1 hour incubation at room temperature, the HTRF signal is measured by the PHERAstar microplate reader. Data analysis is performed in GeneData.

### Example C. CDK1 pNPM T199 In-Cell Western Assay

OVCAR3 cells (ATCC, cat #HTB-161) are seeded at 3×10⁵ cells/mL, or 30,000 cell per well, in 100 μL of 5% FBS RPMI medium overnight in 96-well plates (Corning, cat #07-201-96) at 37° C. with 5% CO₂. The next day, the medium is replaced with 100 μL per well of 5% FBS RPMI containing 20 ng/mL of nocodazole (Cell Signaling, cat #2190S) and incubated for 20 to 21 hours at 37° C. with 5% CO₂ to synchronize the cells in G2/M. After synchronization, the medium is removed. Then, 80 μL of the 3-fold serially diluted compound are added starting at 20 μM (final assay concentration 20 μM) and incubated at 37° C. with 5% CO₂ for another 2 hours. The medium is removed, and the cells are immediately fixed with freshly diluted 4% paraformaldehyde/PBS for 20 minutes at room temperature. The fixing solution is removed, and the cells are washed 3× with PBS containing 0.05% Tween-20 for 5 to 10 minutes per wash with gentle shaking. Next, Odyssey blocking buffer (Licor P/N: 927-60001) with 0.1% Triton X-100 is added at 40 μL per well with 0.1% Triton X-100, and the plates are rocked gently at room temperature for 1 hour. The blocking buffer is removed, and 40 L per well of the primary antibody phosphor-NPM T199 (Cell Signaling, cat #3541S) is added at 1:400 dilution in Odyssey blocking buffer with 0.1% Triton X-100. The plate is then incubated overnight with gentle shaking at 4° C. The next day, the primary antibody is removed, and cells are washed 3× with 140 μL of PBS containing 0.05% Tween-20 for 5 minutes per wash with gentle shaking. The secondary antibody (IRDye® 800CW Goat anti-Rabbit) is then added at a 1:2000 dilution and CellTag 700 stain (Licor CellTaq 700 Stain ICW kit II, cat #P/N 926-41092) at a 1:600 dilution in Odyssey blocking buffer with 0.1% Triton X-100. Plates are rocked gently for 2 hours at room temperature in dark and washed 3× with 140 μL of PBS containing 0.05% Tween-20 for 5 minutes per wash with gentle shaking. During all wash steps, the plate is protected from light. After the final wash, the plate is scanned in both 700 and 800 nm channels on the Odyssey CLx. Data was analyzed with Genedata Screener software using a three or four parameter dose response curve to determine IC₅₀ for each compound.

### Example D. CDK4 pRb S780 HTRF Assay in JeKo-1 Cells

JeKo-1 cells (ATCC, cat #CRL-3006) cultured in 10% FBS RPMI are plated in a 384-well plate (Greiner, cat #784080) at 12,000 cells per well in 8 μL volume. Next, the compound is added at a starting final assay concentration of 10 M and 3-fold serially diluted down to 0.17 nM in 4 μL for a final volume of 12 μL in each well. The plate is incubated for 24 hours at 37° C. with 5% CO₂. Next, 4 μL of 4× Revvity Lysis #2 (Revvity Rb phospho-S780 kit, cat #64RBS780PEH) supplemented with 4× blocking buffer (Revvity Rb phospho-S780 kit, cat #64RBS780PEH) is added to each well. The plate is gently rocked at room temperature for 30 to 45 minutes until cell lysis is completed. To each well of cell lysate, 4 μL of a 1:1 mixture of diluted donor fluorophore solution and diluted acceptor fluorophore solution (Revvity Rb phospho-S780 kit, cat #64RBS780PEH) is added to a final volume of 20 μL per well. The plate is incubated overnight at room temperature in the dark. The HTRF signal is measured by the PHERAstar microplate reader. Data analysis is performed in Genedata.

### Example E. CDK6 pRb S780 HTRF Assay in MV-4-11 Cells

MV-4-11 cells (ATCC, cat #CRL-9591) cultured in 10% FBS RPMI are plated in a 384-well plate (Greiner, cat #784080) at 12,000 cells per well in 8 μL volume. Next, the compound is added at a starting final assay concentration of 10 M and 3-fold serially diluted down to 0.17 nM in 4 μL for a final volume of 12 μL in each well. The plate is then incubated for 24 hours at 37° C. with 5% CO₂. Next, 4 μL of 4× Revvity Lysis #2 (Revvity Rb phospho-S780 kit, cat #64RBS780PEH) supplemented with 4× blocking buffer (Revvity Rb phospho-S780 kit, cat #64RBS780PEH) is added to each well. The plate is gently rocked at room temperature for 30 to 45 minutes until cell lysis was completed. To each well of cell lysate, 4 μL of a 1:1 mixture of diluted donor fluorophore solution and diluted acceptor fluorophore solution (Revvity Rb phospho-S780 kit, cat #64RBS780PEH) are added to a final volume of 20 μL. The plate was incubated overnight at room temperature in the dark. The HTRF signal is measured by the PHERAstar microplate reader. Data analysis is performed in Genedata.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A compound of Formula (I): 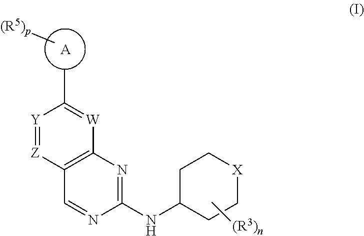
or a pharmaceutically acceptable salt thereof, wherein:
n is 0, 1, 2, 3, 4, 5, or 6;
p is 0, 1, 2, 3, or 4;
W is N or CR¹;
Y is N or CR²;
Z is N or CR⁶;
X is O, S, S(O)₂, S(O)NR^{c}, or N(-L-R⁴);
L is a bond, C₁₋₆ alkylene, C(O), C(O)NR^{a}, C(O)O, S(O)₂, S(O)(═NR^{b}), or S(O)₂NR^{a}, wherein C₁₋₆ alkylene is optionally substituted by 1, 2, 3 or 4 independently selected R^{G} substituents;
R^{a}, R^{b}, and R^{c} are each independently selected from H, C₁₋₆ alkyl, and C₁₋₆ haloalkyl;
or, alternatively, R^{a} and R⁴, taken together with the nitrogen atom to which they are attached, form a 4-7 membered heterocycloalkyl ring or 5-6 membered heteroaryl ring, each of which is optionally substituted by 1, 2, 3, or 4 independently selected R^{4A} substituents;
Ring A is 5-6 membered heteroaryl, having 1 to 2 heteroatom ring members;
R¹ is selected from H, D, halo, CN, C₁₋₆ alkyl, C₁₋₆ haloalkyl, C₃₋₁₀ cycloalkyl, 6-14 membered aryl, 4-15 membered heterocycloalkyl, 5-14 membered heteroaryl, C₃₋₁₀ cycloalkyl-C₁₋₄ alkyl, 6-14 membered aryl-C₁₋₄ alkyl, 4-15 membered heterocycloalkyl-C₁₋₄ alkyl, 5-14 membered heteroaryl-C₁₋₄ alkyl, OR^{b1}, SR^{b1}, NHOR^{a1}, C(O)R^{b1}, C(O)NR^{c1}R^{d1}, C(O)NR^{c1}(OR^{a1}), C(O)OR^{a1}, OC(O)R^{b1}, OC(O)NR^{c1}R^{d1}, NR^{d1}R^{d1}, NR^{c1}NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}C(═NR^{c})R^{b1}, NR^{c1}S(O)NR^{c1}R^{d1}, NR^{c1}S(O)R^{b1}, NR^{c1}S(O)₂R^{b1}, NR^{c1}S(O)(═NR^{c1})R^{b1}, NR^{c1}S(O)₂NR^{c1}R^{d1}, S(O)R^{b1}, S(O)NR^{c1}R^{d1}, S(O)₂R^{b1}, S(O)₂NR^{c1}R^{d1}, OS(O)(═NR^{e1})R^{b1}, OS(O)₂R^{b1}, S(O)(═NR^{e1})R^{b1}, SF₅, P(O)R^{f1}R^{g1}, OP(O)(OR^{h1})(ORⁱ¹), P(O)(OR^{h1})(ORⁱ¹), and BR^{j1}R^{k1}, wherein said C₁₋₆ alkyl, C₁₋₆ haloalkyl, C₃₋₁₀ cycloalkyl, 6-14 membered aryl, 4-15 membered heterocycloalkyl, 5-14 membered heteroaryl, C₃₋₁₀ cycloalkyl-C₁₋₄ alkyl, 6-14 membered aryl-C₁₋₄ alkyl, 4-15 membered heterocycloalkyl-C₁₋₄ alkyl, and 5-14 membered heteroaryl-C₁₋₄ alkyl are each optionally substituted with 1, 2, 3, or 4 independently selected R^{1A} substituents;
each R^{a1}, R^{c1}, and R^{d1} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{1A} substituents;
or, any R^{c1} and R^{d1} attached to the same N atom, together with the N atom to which they are attached, form a 4-10 membered heterocycloalkyl group, wherein the 4-10 membered heterocycloalkyl group is optionally substituted with 1, 2, 3, or 4 independently selected R^{1A} substituents;
each R^{b1} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{1A} substituents;
each R^{e1} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f1} and R^{g1} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h1} and Rⁱ¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j1} and R^{k1} is independently selected from OH, C₁₋₆ alkoxy, and C₁₋₆ haloalkoxy;
or, any R^{j1} and R^{k1} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1A} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{c41})R^{b11}, C(═NR^{c11})NR^{c11}R^{d11}, NR^{c11}C(═NR^{e11})NR^{c11}R^{d11}, NR^{c11}C(═NR^{e11})R^{b11}, NR^{c11}S(O)NR^{c11}R^{d11}, NR^{c11}S(O)R^{b11}, NR^{c11}S(O)₂R^{b11}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, OS(O)₂R^{b11}, S(O)(═NR^{e11})R^{b11}, SF₅, P(O)R^{f11}R^{g11}, OP(O)(OR^{h11})(ORⁱ¹¹), P(O)(OR^{h11})(ORⁱ¹¹) and BR^{j11}R^{k11}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
or, any R^{c11} and R^{d11} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{1B} substituents;
each R^{b11} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1B} substituents;
each R^{c11} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f11} and R^{g11} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h11} and Rⁱ¹¹ is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j11} and R^{k11} is independently selected from OH, C₁₋₆ alkoxy, and C₁₋₆ haloalkoxy;
or, any R^{j11} and R^{k11} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{1B} is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a12}, SR^{a12}, NHOR^{a12}, C(O)R^{b12}, C(O)NR^{c12}R^{d12}, C(O)NR^{c12}(OR^{a12}), C(O)OR^{a12}, OC(O)R^{b12}, OC(O)NR^{c12}R^{d12}, NR^{c12}R^{d12}, NR^{c12}NR^{c12}R^{d12}, NR^{c12}C(O)R^{b12}, NR^{c12}C(O)OR^{a12}, NR^{c12}C(O)NR^{c12}R^{d12}, C(═NR^{e12})R^{b12}, C(═NR^{e12})NR^{c12}R^{d12}, NR^{e12}C(═NR^{e12})NR^{c12}R^{d12}, NR^{c12}C(═NR^{e12})R^{b12}, NR^{c12}S(O)NR^{c12}R^{d12}, NR^{c12}S(O)R^{b12}, NR^{c12}S(O)₂R^{b12}, NR^{c12}S(O)(═NR^{e12})R^{b12}, NR^{c12}S(O)₂NR^{c12}R^{d12}, S(O)R^{b12}, S(O)NR^{c12}R^{d12}, S(O)₂R^{b12}, S(O)₂NR^{c12}R^{d12}, OS(O)(═NR^{e12})R^{b12}, OS(O)₂R^{b12}, S(O)(═NR^{e12})R^{b12}, SF₅, P(O)R^{f12}R^{g12}, OP(O)(OR^{h12})(ORⁱ¹²), P(O)(OR^{h12})(ORⁱ¹²), and BR^{j12}R^{k12}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a12}, R^{c12}, and R^{d12} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
or, any R^{c12} and R^{d12} attached to the same N atom, together with the N atom to which they are attached, form a 4-7 membered heterocycloalkyl group, wherein the 4-7 membered heterocycloalkyl group is optionally substituted with 1, 2, 3, or 4 independently selected R^{G} substituents;
each R^{b12} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{c12} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{f12} and R^{g12} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{h12} and Rⁱ¹² is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{j12} and R^{k12} is independently selected from OH, C₁₋₆ alkoxy, and C₁₋₆ haloalkoxy;
or, any R^{j12} and R^{k12} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R² is selected from H, D, halo, CN, C₁₋₆ alkyl, C₁₋₆ haloalkyl, C₃₋₄ cycloalkyl, C₃₋₄ cycloalkyl-C₁₋₄ alkyl, OR^{b2}, SR^{b2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})R^{c2}R^{d2}, NR^{c2}C(═NR^{e2})NR^{c2}R^{d2}, NR^{c2}C(═NR^{e2})R^{b2}, NR^{c2}S(O)NR^{c2}R^{d2}, NR^{c2}S(O)R^{b2}, NR^{e2}S(O)₂R^{b2}, NR^{e2}S(O)(═NR^{e2})R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}, wherein said C₁₋₆ alkyl, C₁₋₆ haloalkyl, C₃₋₄ cycloalkyl, and C₃₋₄ cycloalkyl-C₁₋₄ alkyl are each optionally substituted with 1, 2, 3, or 4 independently selected R^{G} substituents;
each R^{a2}, R^{c2}, and R^{d2} is independently selected from H, C₁₋₆ alkyl, C₁₋₆ haloalkyl, and C₃₋₄ cycloalkyl, wherein said C₁₋₆ alkyl, C₁₋₆ haloalkyl, and C₃₋₄ cycloalkyl are each optionally substituted with 1, 2, 3, or 4 independently selected R^{G} substituents;
each R^{b2} is independently selected from C₁₋₆ alkyl, C₁₋₆ haloalkyl, and C₃₋₄ cycloalkyl, which are each optionally substituted with 1, 2, 3, or 4 independently selected R^{G} substituents;
each R^{e2} is independently selected from H, OH, CN, C₁₋₆ alkyl, C₁₋₆ alkoxy, C₁₋₆ haloalkyl, and C₁₋₆ haloalkoxy;
each R³ is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}R^{d3}, NR^{c3}NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}C(═NR^{e3})R^{b3}, NR^{c3}S(O)NR^{c3}R^{d3}, NR^{c3}S(O)R^{b3}, NR^{c3}S(O)₂R^{b3}, NR^{c3}S(O)(═NR^{e3})R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R¹³, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a3}, R^{c3}, and R^{d3} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{b3} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e3} is independently selected from H, OH, CN, C₁₋₆ alkyl, C₁₋₆ alkoxy, C₁₋₆ haloalkyl, and C₁₋₆ haloalkoxy;
R⁴ is selected from H, C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted by 1, 2, 3, or 4 independently selected R^{4A} substituents;
each R^{4A} is independently selected from oxo, D, halo, CN, NO₂, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a41}, SR^{a41}, NHOR^{a41}, C(O)R^{b41}, C(O)NR^{c41}R^{d41}, C(O)NR^{c41}(OR^{a41}), C(O)OR^{a41}, OC(O)R^{b41}, OC(O)NR^{c41}R^{d41}, NR^{c41}R^{d41}, NR^{c41}NR^{c41}R^{d41}, NR^{c41}C(O)R^{b41}, NR^{c41}C(O)OR^{a41}, NR^{c41}C(O)NR^{c41}R^{d41}, C(═NR^{e41})R^{b41}, C(═NR^{e41})NR^{c41}R^{d41}, NR^{c41}C(═NR^{e41})NR^{c41}R^{d41}, NR^{c41}C(═NR^{e41})R^{b41}, NR^{c41}S(O)NR^{c41}R^{d41}, NR^{c41}S(O)R^{b41}, NR^{c41}S(O)₂R^{b41}, NR^{c41}S(O)(═NR^{e41})R^{b41}, NR^{c41}S(O)₂NR^{c41}R^{d41}, S(O)R^{b41}, S(O)NR^{c41}R^{d41}, S(O)₂R^{b41}, S(O)₂NR^{c41}R^{d41}, OS(O)(═NR^{e41})R^{b41}, OS(O)₂R^{b41}, and S(O)(═NR^{e41})R^{b41}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{4B} substituents;
each R^{a41}, R^{c41}, and R^{d41} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{4B} substituents;
or, any R^{c41} and R^{d41} attached to the same N atom, together with the N atom to which they are attached, form a 4-7 membered heterocycloalkyl group, which is optionally substituted with 1, 2, 3, or 4 independently selected R^{4B} substituents;
each R^{b41} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{4B} substituents;
each R^{e41} is independently selected from H, OH, CN, C₁₋₆ alkyl, C₁₋₆ alkoxy, C₁₋₆ haloalkyl, C₁₋₆ haloalkoxy, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R^{4B} is independently selected from D, halo, CN, NO₂, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a42}, SR^{a42}, NHOR^{a42}, C(O)R^{b42}, C(O)NR^{c42}R^{d42}, C(O)NR^{c42}(OR^{a42}), C(O)OR^{a42}, OC(O)R^{b42}, OC(O)NR^{c42}R^{d42}, NR^{c42}R^{d42}, NR^{c42}NR^{c42}R^{d42}, NR^{c42}C(O)R^{b42}, NR^{c42}C(O)OR^{a42}, NR^{c42}C(O)NR^{c42}R^{d42}, C(═NR^{e42})R^{b42}, C(═NR^{e42})NR^{c42}R^{d42}, NR^{e42}C(═NR^{e42})NR^{c42}R^{d42}, NR^{c42}C(═NR^{e42})R^{b42}, NR^{c42}S(O)NR^{c42}R^{d42}, NR^{c42}S(O)R^{b42}, NR^{c42}S(O)₂R^{b42}, NR^{c42}S(O)(═NR^{e42})R^{b42}, NR^{c42}S(O)₂NR^{c42}R^{d42}, S(O)R^{b42}, S(O)NR^{c42}R^{d42}, S(O)₂R^{b42}, S(O)₂NR^{c42}R^{d42}, OS(O)(═NR^{e42})R^{b42}, OS(O)₂R^{b42}, and S(O)(═NR^{e42})R^{b42}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a42}, R^{c42}, and R^{d42} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{b42} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{G} substituents;
each R^{e42} is independently selected from H, OH, CN, C₁₋₆ alkyl, C₁₋₆ alkoxy, C₁₋₆ haloalkyl, C₁₋₆ haloalkoxy, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl;
each R⁵ and R⁵¹ is independently selected from oxo, D, halo, NO₂, CN, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a5}, SR^{a5}, NHOR^{a5}, C(O)R^{b5}, C(O)NR^{c5}R^{d5}, C(O)NR^{c5}(OR^{a5}), C(O)OR^{a5}, OC(O)R^{b5}, OC(O)NR^{c5}R^{d5}, NR^{c5}R^{d5}, NR^{c5}NR^{c5}R^{d5}, NR^{c5}C(O)R^{b5}, NR^{c5}C(O)OR^{a5}, NRCC(O)NR^{c5}R^{d5}, C(═NR^{e5})R^{b5}, C(═NR^{e5})NR^{c5}R^{d5}, NR^{c5}C(═NR^{e5})NR^{c5}R^{d5}, NR^{c5}C(═NR^{e5})R^{b5}, NR^{c5}S(O)NR^{c5}R^{d5}, NR^{c5}S(O)R^{b5}, NR^{c5}S(O)₂R^{b5}, NR^{c5}S(O)(═NR^{e5})R^{b5}, NR^{c5}S(O)₂NR^{c5}R^{d5}, S(O)R^{b5}, S(O)NR^{c5}R^{d5}, S(O)₂R^{b5}, S(O)₂NR^{c5}R^{d5}, OS(O)(═NR^{e5})R^{b5}, OS(O)₂R^{b5}, S(O)(═NR^{e5})R^{b5}, SF₅, P(O)R^{f5}R^{g5}, OP(O)(OR^{h5})(ORⁱ⁵), P(O)(OR^{h5})(ORⁱ⁵), and BR^{j5}R^{k5}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted by 1, 2, 3, or 4 independently selected R^{5A} substituents;
or, alternatively, any two R⁵ substituents on adjacent ring members, together with the atoms to which they are attached, form a 4-7 membered cycloalkyl or a 4-7 membered heterocycloalkyl ring, which are each optionally substituted by 1, 2, 3, or 4 independently selected R⁵¹ substituents;
each R^{a5}, R^{c5}, and R^{d5} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected RSA substituents;
or, any R^{c5} and R^{d5} attached to the same N atom, together with the N atom to which they are attached, form a 4-10 membered heterocycloalkyl group, which is optionally substituted with 1, 2, 3, or 4 independently selected R^{5A} substituents;
each R^{b5} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{5A} substituents;
each R^{c5} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f5} and R^{g5} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h5} and Rⁱ⁵ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j5} and R^{k5} is independently selected from OH, C₁₋₆ alkoxy, and C₁₋₆ haloalkoxy;
or, any R^{j5} and R^{k5} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{5A} is independently selected from D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a51}, SR^{a51}, NHOR^{a51}, C(O)R^{b51}, C(O)NR^{c51}R^{d51}, C(O)NR^{c51}(OR^{a51}), C(O)OR^{a51}, OC(O)R^{b51}, OC(O)NR^{c51}R^{d51}, NR^{c51}R^{d51}, NR^{c51}NR^{c51}R^{d51}, NR^{c51}C(O)R^{b51}, NR^{c51}C(O)OR^{a51}, NR^{c51}C(O)NR^{c51}R^{d51}, C(═NR^{e51})R^{b51}, C(═NR^{e51})NR^{c51}R^{d51}, NR^{c51}C(═NR^{e51})NR^{c51}R^{d51}, NR^{c51}C(═NR^{e51})R^{b51}, NR^{c51}S(O)NR^{c51}R^{d51}, NR^{c51}S(O)R^{b51}, NR^{c51}S(O)₂R^{b51}, NR^{c51}S(O)(═NR^{e51})R^{b51}, NR^{c51}S(O)₂NR^{c51}R^{d51}, S(O)R^{b51}, S(O)NR^{c51}R^{d51}, S(O)₂R^{b51}, S(O)₂NR^{c51}R^{d51}, OS(O)(═NR^{e51})R^{b51}, OS(O)₂R^{b51}, S(O)(═NR^{e51})R^{b51}, SF₅, P(O)R^{f51}R^{g51}, OP(O)(OR^{h51})(ORⁱ⁵¹), P(O)(OR^{h51})(ORⁱ⁵¹), and BR^{j51}R^{k51}, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{G} substituents;
each R^{a51}, R^{c51}, and R^{d51} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted with 1, 2, 3, or 4 independently selected R^{G} substituents;
or, any R^{c51} and R^{d51} attached to the same N atom, together with the N atom to which they are attached, form a 4-7 membered heterocycloalkyl group, which is optionally substituted with 1, 2, 3, or 4 independently selected R^{G} substituents;
each R^{b51} is independently selected from C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl, which are each optionally substituted with 1, 2, 3, or 4 independently selected R^{G} substituents;
each R^{e51} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{f51} and R^{g51} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{h51} and Rⁱ⁵¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl;
each R^{j51} and R^{k51} is independently selected from OH, C₁₋₆ alkoxy, and C₁₋₆ haloalkoxy;
or, any R^{j51} and R^{k51} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
R⁶ is selected from H, D, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino; and
each R^{G} is independently selected from OH, NO₂, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₄ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

2. The compound of claim 1, or a pharmaceutically acceptable salt thereof, wherein n is 0, 1, or 2.

3. The compound of claim 1, or a pharmaceutically acceptable salt thereof, wherein n is 1.

4. The compound of claim 1, or a pharmaceutically acceptable salt thereof, wherein p is 0, 1, or 2.

5. The compound of claim 1, or a pharmaceutically acceptable salt thereof, wherein W is N.

6. The compound of claim 1, or a pharmaceutically acceptable salt thereof, wherein W is CR¹.

7. The compound of claim 1, or a pharmaceutically acceptable salt thereof, wherein Y is N.

8. The compound of claim 1, or a pharmaceutically acceptable salt thereof, wherein Y is CR².

9. The compound of claim 1, or a pharmaceutically acceptable salt thereof, wherein Z is N.

10. The compound of claim 1, or a pharmaceutically acceptable salt thereof, wherein Z is CR⁶.

11. The compound of claim 1, or a pharmaceutically acceptable salt thereof, wherein X is O.

12. The compound of claim 1, or a pharmaceutically acceptable salt thereof, wherein X is N(-L-R⁴).

13. The compound of claim 1, or a pharmaceutically acceptable salt thereof, wherein X is O or N(-L-R⁴).

14. The compound of claim 1, or a pharmaceutically acceptable salt thereof, wherein L is a bond.

15. The compound of claim 1, or a pharmaceutically acceptable salt thereof, wherein L is C(O).

16. The compound of claim 1, or a pharmaceutically acceptable salt thereof, wherein L is C(O)O.

17. The compound of claim 1, or a pharmaceutically acceptable salt thereof, wherein L is S(O)₂.

18. The compound of claim 1, or a pharmaceutically acceptable salt thereof, wherein L is S(O)₂NR^{a}, wherein R^{a} is H, C₁₋₆ alkyl, or C₁₋₆ haloalkyl, or, alternatively, R^{a} and R⁴, taken together with the nitrogen atom to which they are attached, form a 4-7 membered heterocycloalkyl ring or 5-6 membered heteroaryl ring, each of which is optionally substituted by 1, 2, 3, or 4 independently selected R^{4A} substituents.

19. The compound of claim 1, or a pharmaceutically acceptable salt thereof, wherein Ring A is a 5-6 membered heteroaryl, having 1 to 2 heteroatom ring members, wherein at least 1 heteroatom is N.

20. The compound of claim 1, or a pharmaceutically acceptable salt thereof, wherein Ring moiety A is a pyrazole ring, an imidazole ring, a thiazole ring, an isothiazole ring, an oxazole ring, an isoxazole ring, a pyridine ring, or a pyrimidine ring.

21. The compound of claim 1, or a pharmaceutically acceptable salt thereof, wherein Ring moiety A is a pyrazole ring, an imidazole ring, a thiazole ring, or a pyridine ring.

22. The compound of claim 1, or a pharmaceutically acceptable salt thereof, wherein R¹ is selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{b1}, SR^{b1}, C(O)R^{b1}, C(O)NR_{c1}R^{d1}, C(O)OR^{a1}, OC(O)R^{b1}, OC(O)NR_{c1}R^{d1}, NR^{c1}R^{d1}, NR^{c1}C(O)R^{b1}, NR^{c1}C(O)OR^{a1}, NR^{c1}C(O)NR^{c1}R^{a1}, NR^{c1}S(O)₂R^{b1}, NR^{c1}S(O)₂NR^{c1}R^{c1}, S(O)₂R^{b1}, and S(O)₂NR^{c1}R^{d1}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents.

23. The compound of claim 1, or a pharmaceutically acceptable salt thereof, wherein R¹ is selected from H, halo, CN, C₁₋₆ alkyl, C₁₋₆ haloalkyl, C₃₋₇ cycloalkyl, phenyl, 4-7 membered heterocycloalkyl, 5-6 membered heteroaryl, and OR^{b1}, wherein said C₁₋₆ alkyl, C₁₋₆ haloalkyl, C₃₋₇ cycloalkyl, phenyl, 4-7 membered heterocycloalkyl, and 5-6 membered heteroaryl are each optionally substituted with 1 or 2 independently selected R^{1A} substituents.

24. The compound of claim 1, or a pharmaceutically acceptable salt thereof, wherein:
each R^{a1}, R^{c1}, and R^{d1} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
each R^{b1} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1A} substituents;
each R^{1A} is independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄alkyl, OR^{a11}, SR^{a11}, C(O)R^{b11}, C(O)NR^{c11}R^{d11}, C(O)OR^{a11}, OC(O)R^{b11}OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{d11}R^{d11}, S(O)₂R^{b11}, and S(O)₂NR^{c11}R^{d11}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{1B} substituents;
each R^{b11} is independently selected from C₁₋₆ alkyl and C₁₋₆ haloalkyl, which are each optionally substituted with 1, 2, 3, or 4 independently selected R^{1B} substituents; and
each R^{1B} is independently selected from OH, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₄ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

25. The compound of claim 1, or a pharmaceutically acceptable salt thereof, wherein R² is selected from H, D, halo, CN, C₁₋₆ alkyl, C₁₋₆ haloalkyl, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)₂R^{b2}, NR^{e2}S(O)₂NR^{c2}R^{d2}, S(O)₂R^{b2}, and S(O)₂NR^{c2}R^{d2}, wherein said C₁₋₆ alkyl and C₁₋₆ haloalkyl are each optionally substituted with 1, 2, 3, or 4 independently selected R^{G} substituents.

26. The compound of claim 1, or a pharmaceutically acceptable salt thereof, wherein R² is selected from H, D, halo, CN, C₁₋₆ alkyl, and C₁₋₆ haloalkyl.

27. The compound of claim 1, or a pharmaceutically acceptable salt thereof, wherein each R^{a2}, R^{c2}, and R^{d2} is independently selected from H, C₁₋₆ alkyl, C₁₋₆ haloalkyl, and C₃₋₄ cycloalkyl; and each R^{b2} is independently selected from C₁₋₆ alkyl, C₁₋₆ haloalkyl, and C₃₋₄ cycloalkyl.

28. The compound of claim 1, or a pharmaceutically acceptable salt thereof, wherein each R³ is independently selected from —C₁₋₄ alkylene-OR^{a3}, —C₁₋₄ alkylene-NR^{c3}R^{d3}, OR^{a3}, and NR^{c3}R^{d3}.

29. The compound of claim 1, or a pharmaceutically acceptable salt thereof, wherein each R³ is independently selected from —C₁₋₄ alkylene-OH and OH.

30. The compound of claim 1, or a pharmaceutically acceptable salt thereof, wherein each R^{a3}, R^{c3}, and R^{d3} is independently selected from H, C₁₋₆ alkyl, and C₁₋₆ haloalkyl; and each R^{b3} is independently selected from C₁₋₆ alkyl and C₁₋₆ haloalkyl.

31. The compound of claim 1, or a pharmaceutically acceptable salt thereof, wherein R⁴ is selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{4A} substituents.

32. The compound of claim 1, or a pharmaceutically acceptable salt thereof, wherein R⁴ is selected from H, C₁₋₆ alkyl, C₁₋₆ haloalkyl, C₃₋₇ cycloalkyl, phenyl, 4-7 membered heterocycloalkyl, and 5-6 membered heteroaryl, wherein said C₁₋₆ alkyl, C₁₋₆ haloalkyl, C₃₋₇ cycloalkyl, phenyl, 4-7 membered heterocycloalkyl, and 5-6 membered heteroaryl are each optionally substituted by 1 or 2 independently selected R^{4A} substituents.

33. The compound of claim 1, or a pharmaceutically acceptable salt thereof, wherein:
each R^{4A} is independently selected from oxo, halo, CN, C₁₋₆ alkyl, C₁₋₆ haloalkyl, C₃₋₇ cycloalkyl, phenyl, 4-7 membered heterocycloalkyl, 5-6 membered heteroaryl, OR^{a41}, SR^{a41}, C(O)R^{b41}, C(O)NR^{c41}R^{d41}, C(O)OR^{a41}, OC(O)R^{b41}, OC(O)NR^{c41}R^{d41}, NR^{c41}R^{d41}, NR^{c41}C(O)R^{b41}, NR^{c41}C(O)OR^{a41}, NR^{c41}C(O)NR^{c41}R^{d41}, NR^{c41}S(O)₂R^{b41}, NR^{c41}S(O)₂NR^{c41}R^{d41}, S(O)₂R^{b41}, and S(O)₂NR^{c41}R^{d41}, wherein said C₁₋₆ alkyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R^{4B} substituents;
each R^{a41}, R^{c41}, and R^{d41} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{4B} substituents;
each R^{b41} is independently selected from C₁₋₆ alkyl and C₁₋₆ haloalkyl, which are each optionally substituted with 1, 2, 3, or 4 independently selected R^{4B} substituents;
each R^{4B} is independently selected from halo, CN, C₁₋₆ alkyl, C₁₋₆ haloalkyl, OR^{a42}, C(O)R^{b42}, C(O)NR^{c42}R^{d42}, C(O)OR^{a42}, NR^{c42}R^{d42}, NR^{c42}C(O)R^{b42}, S(O)₂R^{b42}, and S(O)₂NR^{c42}R^{d42};
each R^{a42}, R^{c42}, and R^{d42} is independently selected from H, C₁₋₆ alkyl, and C₁₋₆ haloalkyl; and
each R^{b42} is independently selected from C₁₋₆ alkyl and C₁₋₆ haloalkyl.

34. The compound of claim 1, or a pharmaceutically acceptable salt thereof, wherein:
each R⁵ and R⁵¹ is independently selected from oxo, D, halo, CN, C₁₋₆ alkyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, 5-10 membered heteroaryl-C₁₋₄ alkyl, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R¹⁵, and S(O)₂NR^{c5}R^{d5}, wherein said C₁₋₆ alkyl, C₂₋₆ alkynyl, C₁₋₆ haloalkyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl, 6-10 membered aryl-C₁₋₄ alkyl, 4-10 membered heterocycloalkyl-C₁₋₄ alkyl, and 5-10 membered heteroaryl-C₁₋₄ alkyl are each optionally substituted by 1, 2, 3, or 4 independently selected R^{5A} substituents;
or, alternatively, any two R⁵ substituents on adjacent ring members, together with the atoms to which they are attached, form a 4-7 membered cycloalkyl or a 4-7 membered heterocycloalkyl ring, which are each optionally substituted by 1, 2, 3, or 4 independently selected R⁵¹ substituents.

35. The compound of claim 1, or a pharmaceutically acceptable salt thereof, wherein:
each R⁵ and R⁵¹ is independently selected from oxo, D, halo, CN, C₁₋₆ alkyl, C₂₋₆ alkynyl, C₁₋₆ haloalkyl, C₃₋₆ cycloalkyl, phenyl, 4-7 membered heterocycloalkyl, C₃₋₇ cycloalkyl-C₁₋₄ alkyl, OR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NRCC(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5}, wherein said C₁₋₆ alkyl, C₂₋₆ alkynyl, C₁₋₆ haloalkyl, C₃₋₆ cycloalkyl, phenyl, and 4-7 membered heterocycloalkyl are each optionally substituted by 1 or 2 independently selected R^{5A} substituents;
or, alternatively, any two R⁵ substituents on adjacent ring members, together with the atoms to which they are attached, form a 4-7 membered cycloalkyl or a 4-7 membered heterocycloalkyl ring, which are each optionally substituted by 1, 2, 3, or 4 independently selected R⁵¹ substituents.

36. The compound of claim 1, or a pharmaceutically acceptable salt thereof, wherein:
each R^{a5}, R^{c5}, and R^{d5} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{5A} substituents;
each R^{b5} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{5A} substituents;
each R^{5A} is independently selected from D, halo, CN, C₁₋₆ alkyl, C₁₋₆ haloalkyl, C₃₋₅ cycloalkyl, 4-7 membered heterocycloalkyl-C₁₋₄ alkyl, OR^{a51}, SR^{a51}, C(O)R^{b51}, C(O)NR^{c51}R^{d51}, C(O)OR^{a51}, OC(O)R^{b51}, OC(O)NR^{c51}R^{d51}, NR^{c51}R^{d51}, NR^{c51}C(O)R^{b51}, NR^{c51}C(O)OR^{a51}, NR^{c1}C(O)NR^{c51}R^{d51}, NR^{c51}S(O)₂R^{b51}, NR^{c51}S(O)₂NR^{c51}R^{d51}, S(O)₂R^{b51}, and S(O)₂NR^{c51}R^{d51}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{G} substituents;
each R^{a51}, R^{c51}, and R^{d51} is independently selected from H, C₁₋₆ alkyl, and C₁₋₆ haloalkyl; and
each R^{b51} is independently selected from C₁₋₆ alkyl and C₁₋₆ haloalkyl.

37. The compound of claim 1, or a pharmaceutically acceptable salt thereof, wherein:
each R^{a5}, R^{c}, and R^{d5} is independently selected from H, C₁₋₆ alkyl, C₁₋₆ haloalkyl, C₃₋₅ cycloalkyl, and 4-6 membered heterocycloalkyl are each optionally substituted with 1, 2, 3, or 4 independently selected R^{5A} substituents;
each R^{b5} is independently selected from C₁₋₆ alkyl and C₁₋₆ haloalkyl, which are each optionally substituted with 1, 2, 3, or 4 independently selected R^{5A} substituents;
each R^{5A} is independently selected from D, halo, CN, C₁₋₆ alkyl, C₁₋₆ haloalkyl, 4-7 membered heterocycloalkyl-C₁₋₄ alkyl, OR^{a51}, and NR^{c51}R^{d51}; and
each R^{a51}, R^{c51}, and R^{d51} is independently selected from H, C₁₋₆ alkyl, and C₁₋₆ haloalkyl.

38. The compound of claim 1, or a pharmaceutically acceptable salt thereof, wherein R⁶ is selected from H, D, CN, halo, C₁₋₃ alkyl, and C₁₋₃ haloalkyl.

39. The compound of claim 1, or a pharmaceutically acceptable salt thereof, wherein each R^{G} is independently selected from OH, NO₂, CN, halo, C₁₋₃ alkyl, and C₁₋₃ haloalkyl.

40. The compound of claim 1, or a pharmaceutically acceptable salt thereof, wherein:
n is 0, 1, or 2;
p is 0, 1, or 2;
W is N or CR¹;
Y is N or CR²;
Z is N or CR⁶;
X is O, S(O)₂, or N(-L-R⁴);
L is a bond, C₁₋₆ alkylene, C(O), C(O)O, S(O)₂, or S(O)₂NR^{a};
R^{a} is selected from H, C₁₋₆ alkyl, and C₁₋₆ haloalkyl;
or, alternatively, R^{a} and R⁴, taken together with the nitrogen atom to which they are attached, form a 4-7 membered heterocycloalkyl ring or 5-6 membered heteroaryl ring, each of which is optionally substituted by 1, 2, 3, or 4 independently selected R^{4A} substituents;
Ring moiety A is a pyrazole ring, an imidazole ring, a thiazole ring, an isothiazole ring, an oxazole ring, an isoxazole ring, a pyridine ring, or a pyrimidine ring;
R¹ is selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{b1}, SR^{b1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NRCC(O)R^{b1}, NR^{c1}C(O)OR^{a1}, NRCC(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents.
each R^{a1}, R^{c1}, and R^{d1} is independently selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1A} substituents;
each R^{b1} is independently selected from C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, 3, or 4 independently selected R^{1A} substituents;
each R^{1A} is independently selected from H, D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a11}, SR^{a11}, C(O)R^{b11}, C(O)NR^{c11}R^{d11}, C(O)OR^{a11}, OC(O)R^{b11}OC(O)NR^{c11}R^{d11}, NR^{c11}R^{d11}, NR^{c11}C(O)R^{b11}, NR^{c11}C(O)OR^{a11}, NR^{c11}C(O)NR^{c11}R^{d11}, NR^{c11}S(O)₂R^{b11}, NR^{c11}S(O)₂NR^{c11}R^{d11}, S(O)₂R^{b11}, and S(O)₂NR^{c11}R^{d11}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, 3, or 4 independently selected R^{1B} substituents;
each R^{a11}, R^{c11}, and R^{d11} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{1B} substituents;
each R^{b11} is independently selected from C₁₋₆ alkyl and C₁₋₆ haloalkyl, which are each optionally substituted with 1, 2, 3, or 4 independently selected R^{1B} substituents;
each R^{1B} is independently selected from OH, CN, halo, C₁₋₃ alkyl, C₂₋₃ alkenyl, C₂₋₃ alkynyl, C₁₋₃ haloalkyl, cyano-C₁₋₃ alkyl, HO—C₁₋₃ alkyl, C₁₋₃ alkoxy-C₁₋₃ alkyl, C₃₋₄ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
R² is selected from H, D, halo, CN, C₁₋₆ alkyl, and C₁₋₆ haloalkyl;
each R³ is independently selected from D, halo, CN, C₁₋₆ alkyl, C₁₋₆ haloalkyl, OR^{a3}, and NR^{c3}R^{d3} wherein said C₁₋₆ alkyl and C₁₋₆ haloalkyl are each optionally substituted with 1 or 2 independently selected R^{G} substituents;
each R^{a3}, R^{c3}, and R^{d3} is independently selected from H, C₁₋₆ alkyl, and C₁₋₆ haloalkyl;
each R^{b3} is independently selected from C₁₋₆ alkyl and C₁₋₆ haloalkyl;
R⁴ is selected from H, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{4A} substituents;
each R^{4A} is independently selected from oxo, halo, CN, C₁₋₆ alkyl, C₁₋₆ haloalkyl, C₃₋₇ cycloalkyl, phenyl, 4-7 membered heterocycloalkyl, 5-6 membered heteroaryl, OR^{a41}, SR^{a41}, C(O)R^{b41}, C(O)NR^{c41}R^{d41}, C(O)OR^{a41}, OC(O)R^{b41}, OC(O)NR^{c41}R^{d41}, NR^{c41}R^{d41}, NR^{c41}C(O)R^{b41}, NR^{c41}C(O)OR^{a41}, NR^{c41}C(O)NR^{c41}R^{d41}, NR^{c41}S(O)₂R^{b41}, NR^{c41}S(O)₂NR^{c41}R^{d41}, S(O)₂R^{b41}, and S(O)₂NR^{c41}R^{d41}, wherein said C₁₋₆ alkyl, C₁₋₆ haloalkyl, C₃₋₇ cycloalkyl, phenyl, 4-7 membered heterocycloalkyl, and 5-6 membered heteroaryl are each optionally substituted with 1, 2, 3, or 4 independently selected R^{4B} substituents;
each R^{a41}, R^{c41}, and R^{d41} is independently selected from H, C₁₋₆ alkyl, and C₁₋₆ haloalkyl, wherein said C₁₋₆ alkyl and C₁₋₆ haloalkyl are each optionally substituted with 1, 2, 3, or 4 independently selected R^{4B} substituents;
each R^{b41} is independently selected from C₁₋₆ alkyl and C₁₋₆ haloalkyl, which are each optionally substituted with 1, 2, 3, or 4 independently selected R^{4B} substituents;
each R^{4B} is independently selected from halo, CN, C₁₋₆ alkyl, C₁₋₆ haloalkyl, OR^{a42}, C(O)R^{b42}, C(O)NR^{c42}R^{d42}, C(O)OR^{a42}, NR^{c42}R^{d42}, NR^{c42}C(O)R^{b42}, S(O)₂R^{b42}, and S(O)₂NR^{c42}R^{d42};
each R^{a42}, R^{c42}, and R^{d42} is independently selected from H, C₁₋₆ alkyl, and C₁₋₆ haloalkyl;
each R^{b42} is independently selected from C₁₋₆ alkyl and C₁₋₆ haloalkyl;
each R⁵ is independently selected from oxo, D, halo, CN, C₁₋₆ alkyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5}, wherein said C₁₋₆ alkyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{5A} substituents;
each R⁵¹ is independently selected from D, halo, CN, C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, 5-6 membered heteroaryl-C₁₋₄ alkyl, OR^{a5}, SR^{a5}, C(O)R^{b5}, C(O)NR^{c5}R^{d5}, C(O)OR^{a5}, OC(O)R^{b5}, OC(O)NR^{c5}R^{d5}, NR^{c5}R^{d5}, NR^{c5}C(O)R^{b5}, NR^{c5}C(O)OR^{a5}, NR^{c5}C(O)NR^{c5}R^{d5}, NR^{c5}S(O)₂R^{b5}, NR^{c5}S(O)₂NR^{c5}R^{d5}, S(O)₂R^{b5}, and S(O)₂NR^{c5}R^{d5}, wherein said C₁₋₆ alkyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted by 1, 2, 3, or 4 independently selected R^{5A} substituents;
or, alternatively, any two R⁵ substituents on adjacent ring members, together with the atoms to which they are attached, form a 4-7 membered cycloalkyl or a 4-7 membered heterocycloalkyl ring, which are each optionally substituted by 1, 2, 3, or 4 independently selected R⁵¹ substituents;
each R^{a5}, R^{c5}, and R^{d5} is independently selected from H, C₁₋₆ alkyl, C₁₋₆ haloalkyl, C₃₋₅ cycloalkyl, and 4-6 membered heterocycloalkyl, which are each optionally substituted with 1, 2, 3, or 4 independently selected R^{5A} substituents;
each R^{b5} is independently selected from C₁₋₆ alkyl and C₁₋₆ haloalkyl, which are each optionally substituted with 1, 2, 3, or 4 independently selected R^{5A} substituents;
each R^{5A} is independently selected from D, halo, CN, C₁₋₆ alkyl, C₁₋₆ haloalkyl, 4-7 membered heterocycloalkyl-C₁₋₄ alkyl, OR^{a51}, and NR^{c51}R^{d51};
each R^{a51}, R^{c51}, and R^{d51} is independently selected from H, C₁₋₆ alkyl, and C₁₋₆ haloalkyl;
R⁶ is selected from H, D, CN, halo, C₁₋₃ alkyl, and C₁₋₃ haloalkyl; and
each R^{G} is independently selected from OH, CN, halo, C₁₋₃ alkyl, C₁₋₃ haloalkyl, cyano-C₁₋₃ alkyl, HO—C₁₋₃ alkyl, C₁₋₃ alkoxy-C₁₋₃ alkyl, C₃₋₄ cycloalkyl, C₁₋₃ alkoxy, C₁₋₃ haloalkoxy, amino, C₁₋₃ alkylamino, di(C₁₋₃ alkyl)amino, C₁₋₃ alkylsulfonyl, carbamyl, C₁₋₃ alkylcarbamyl, di(C₁₋₃ alkyl)carbamyl, C₁₋₃ alkylcarbonyl, C₁₋₃ alkoxycarbonyl, C₁₋₃ alkylcarbonyloxy, and C₁₋₃ alkylcarbonylamino.

41. The compound of claim 41, or a pharmaceutically acceptable salt thereof, wherein each R³ is independently selected from —C₁₋₄ alkylene-OR^{a3}, —C₁₋₄ alkylene-NR^{c3}R^{d3}, OR^{a3}, and NR^{c3}R^{d3}.

42. The compound of claim 1, wherein the compound is a compound of Formula (Ia): 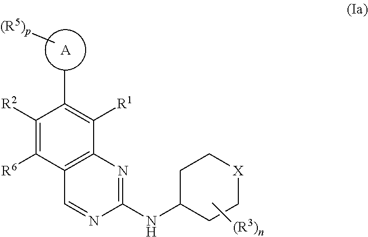
or a pharmaceutically acceptable salt thereof.

43. The compound of claim 1, wherein the compound is a compound of Formula (Ib): 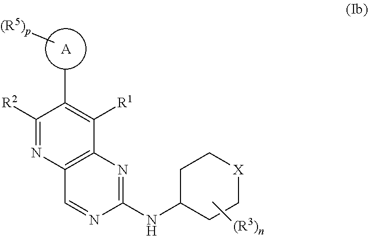
or a pharmaceutically acceptable salt thereof.

44. The compound of claim 1, wherein the compound is a compound of Formula (Ic): 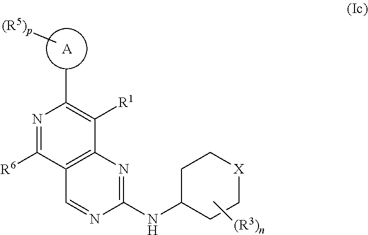
or a pharmaceutically acceptable salt thereof.

45. The compound of claim 1, selected from:
(3R,4R)-4-((7-(1H-pyrazol-4-yl)quinazolin-2-yl)amino)-1-(methylsulfonyl)piperidin-3-ol;
(3R,4R)-4-((7-(1H-pyrazol-4-yl)pyrido[3,2-d]pyrimidin-2-yl)amino)-1-(methylsulfonyl)piperidin-3-ol;
(3R,4R)-4-((7-(3-cyclopropyl-1H-pyrazol-4-yl)pyrido[4,3-d]pyrimidin-2-yl)amino)-1-(methylsulfonyl)piperidin-3-ol;
(3R,4R)-4-((6-chloro-7-(1H-pyrazol-4-yl)quinazolin-2-yl)amino)-1-(methylsulfonyl)piperidin-3-ol;
(3R,4R)-4-((7-(3-methyl-1H-pyrazol-4-yl)quinazolin-2-yl)amino)-1-(methylsulfonyl)piperidin-3-ol;
4-(2-(((3R,4R)-3-hydroxy-1-(methylsulfonyl)piperidin-4-yl)amino)quinazolin-7-yl)pyridin-2(1H)-one;
(3R,4R)-4-((7-(1H-imidazol-4-yl)quinazolin-2-yl)amino)-1-(methylsulfonyl)piperidin-3-ol;
(3R,4R)-4-((7-(1H-pyrazol-4-yl)quinazolin-2-yl)amino)-1-((1-methyl-1H-pyrazol-4-yl)sulfonyl)piperidin-3-ol;
(3R,4R)-4-((7-(3-methyl-1H-pyrazol-4-yl)quinazolin-2-yl)amino)-1-((1-methyl-1H-pyrazol-4-yl)sulfonyl)piperidin-3-ol;
(3R,4R)-1-(cyclopropylsulfonyl)-4-((7-(3-methyl-1H-pyrazol-4-yl)quinazolin-2-yl)amino)piperidin-3-ol;
(3R,4R)-1-((3-methoxy-1-methyl-1H-pyrazol-4-yl)sulfonyl)-4-((7-(3-methyl-1H-pyrazol-4-yl)quinazolin-2-yl)amino)piperidin-3-ol;
Methyl (3R,4R)-3-hydroxy-4-((7-(3-methyl-1H-pyrazol-4-yl)quinazolin-2-yl)amino)piperidine-1-carboxylate;
(3R,4R)-4-((7-(1H-pyrazol-4-yl)quinazolin-2-yl)amino)-1-(cyclopropylsulfonyl)piperidin-3-ol;
1-((3R,4R)-3-hydroxy-4-((7-(3-methyl-1H-pyrazol-4-yl)quinazolin-2-yl)amino)piperidin-1-yl)ethan-1-one;
(3R,4R)-4-((7-(3-cyclopropyl-1H-pyrazol-4-yl)quinazolin-2-yl)amino)-1-(pyridin-3-ylmethyl)piperidin-3-ol;
(3R,4R)-4-((7-(3-(2,2-difluoroethoxy)-1H-pyrazol-4-yl)quinazolin-2-yl)amino)-1-(methylsulfonyl)piperidin-3-ol;
(3S,4R)-4-((8-(3-chloro-4-((dimethylamino)methyl)phenyl)-7-(1H-pyrazol-4-yl)quinazolin-2-yl)amino)tetrahydro-2H-pyran-3-ol;
(3S,4R)-4-((8-(4-methylpiperazin-1-yl)-7-(1H-pyrazol-4-yl)quinazolin-2-yl)amino)tetrahydro-2H-pyran-3-ol;
((3R,4R)-4-((8-isopropoxy-7-(1-methyl-1H-pyrazol-4-yl)quinazolin-2-yl)amino)-1-(methylsulfonyl)piperidin-3-yl)methanol;
(3R,4R)-1-(methylsulfonyl)-4-((7-(pyridin-3-yl)quinazolin-2-yl)amino)piperidin-3-ol;
(3R,4R)-1-(methylsulfonyl)-4-((7-(thiazol-5-yl)quinazolin-2-yl)amino)piperidin-3-ol;
(3R,4R)-4-((7-(6,7-dihydro-5H-pyrazolo[5,1-b][1,3]oxazin-3-yl)quinazolin-2-yl)amino)-1-(methylsulfonyl)piperidin-3-ol;
(3R,4R)-1-((3,3-difluoroazetidin-1-yl)sulfonyl)-4-((7-(3-methyl-1H-pyrazol-4-yl)quinazolin-2-yl)amino)piperidin-3-ol;
(3R,4R)-4-((7-(3-cyclopropyl-1H-pyrazol-4-yl)-6,8-difluoroquinazolin-2-yl)amino)-1-(methylsulfonyl)piperidin-3-ol;
2-((3R,4R)-4-((7-(3-cyclopropyl-1H-pyrazol-4-yl)quinazolin-2-yl)amino)-3-hydroxypiperidin-1-yl)acetamide; and
(S)-5-(((3R,4R)-4-((7-(3-cyclopropyl-1H-pyrazol-4-yl)quinazolin-2-yl)amino)-3-hydroxypiperidin-1-yl)methyl)pyrrolidin-2-one;
or a pharmaceutically acceptable salt thereof.

46. The compound of claim 1, selected from:
(3R,4R)-1-((1-cyclopropyl-1H-pyrazol-3-yl)sulfonyl)-4-((7-(3-methyl-1H-pyrazol-4-yl)quinazolin-2-yl)amino)piperidin-3-ol;
methyl (3R,4R)-4-((7-(3-cyclopropyl-1H-pyrazol-4-yl)quinazolin-2-yl)amino)-3-hydroxypiperidine-1-carboxylate;
methyl (3R,4R)-4-((7-(3-ethyl-1H-pyrazol-4-yl)quinazolin-2-yl)amino)-3-hydroxypiperidine-1-carboxylate;
1-((3R,4R)-4-((7-(3-cyclopropyl-1H-pyrazol-4-yl)quinazolin-2-yl)amino)-3-hydroxypiperidin-1-yl)ethan-1-one;
(3S,4R)-4-((7-(1H-pyrazol-4-yl)quinazolin-2-yl)amino)tetrahydro-2H-pyran-3-ol;
(3S,4R)-4-((7-(1H-pyrazol-4-yl)quinazolin-2-yl)amino)-1-(methylsulfonyl)piperidin-3-ol;
rac-((3R,4R)-4-((7-(1H-pyrazol-4-yl)quinazolin-2-yl)amino)-1-(methylsulfonyl)piperidin-3-yl)methanol;
(3R,4R)-4-((7-(3-isopropyl-1H-pyrazol-4-yl)quinazolin-2-yl)amino)-1-(methylsulfonyl)piperidin-3-ol;
(3R,4R)-4-((7-(3-cyclopropyl-1H-pyrazol-4-yl)quinazolin-2-yl)amino)-1-(methylsulfonyl)piperidin-3-ol;
(3R,4R)-4-((7-(3-fluoro-1H-pyrazol-4-yl)quinazolin-2-yl)amino)-1-(methylsulfonyl)piperidin-3-ol;
(3R,4R)-4-((7-(3-methoxy-1H-pyrazol-4-yl)quinazolin-2-yl)amino)-1-(methylsulfonyl)piperidin-3-ol;
(3R,4R)-4-((7-(1-methyl-1H-imidazol-4-yl)quinazolin-2-yl)amino)-1-(methylsulfonyl)piperidin-3-ol;
(3R,4R)-4-((7-(1-methyl-1H-imidazol-5-yl)quinazolin-2-yl)amino)-1-(methylsulfonyl)piperidin-3-ol;
(3R,4R)-1-(methylsulfonyl)-4-((7-(pyridin-4-yl)quinazolin-2-yl)amino)piperidin-3-ol;
(3R,4R)-1-(methylsulfonyl)-4-((7-(pyridazin-4-yl)quinazolin-2-yl)amino)piperidin-3-ol;
(3R,4R)-4-((7-(isothiazol-4-yl)quinazolin-2-yl)amino)-1-(methylsulfonyl)piperidin-3-ol;
(3R,4R)-1-(methylsulfonyl)-4-((7-(3-(trifluoromethyl)-1H-pyrazol-4-yl)quinazolin-2-yl)amino)piperidin-3-ol;
4-(2-(((3R,4R)-3-hydroxy-1-(methylsulfonyl)piperidin-4-yl)amino)quinazolin-7-yl)-1H-pyrazole-3-carbonitrile;
(3R,4R)-4-((7-(3-ethyl-1H-pyrazol-4-yl)quinazolin-2-yl)amino)-1-(methylsulfonyl)piperidin-3-ol;
(3R,4R)-4-((7-(1-methyl-1H-pyrazol-5-yl)quinazolin-2-yl)amino)-1-(methylsulfonyl)piperidin-3-ol;
(3R,4R)-4-((7-(1H-pyrazol-5-yl)quinazolin-2-yl)amino)-1-(methylsulfonyl)piperidin-3-ol;
(3R,4R)-4-((7-(1-cyclopropyl-1H-pyrazol-5-yl)quinazolin-2-yl)amino)-1-(methylsulfonyl)piperidin-3-ol;
(3R,4R)-4-((7-(1-ethyl-1H-pyrazol-5-yl)quinazolin-2-yl)amino)-1-(methylsulfonyl)piperidin-3-ol;
(3R,4R)-4-((7-(1-ethyl-1H-imidazol-5-yl)quinazolin-2-yl)amino)-1-(methylsulfonyl)piperidin-3-ol;
(3R,4R)-4-((7-(1-cyclopropyl-1H-imidazol-5-yl)quinazolin-2-yl)amino)-1-(methylsulfonyl)piperidin-3-ol;
(3R,4R)-4-((7-(3-fluoro-1-methyl-1H-pyrazol-4-yl)quinazolin-2-yl)amino)-1-(methylsulfonyl)piperidin-3-ol;
(3R,4R)-4-((7-(5-fluoro-1-methyl-1H-pyrazol-4-yl)quinazolin-2-yl)amino)-1-(methylsulfonyl)piperidin-3-ol;
(3R,4R)-1-(methylsulfonyl)-4-((7-(3-(tetrahydro-2H-pyran-4-yl)-1H-pyrazol-4-yl)quinazolin-2-yl)amino)piperidin-3-ol;
(3R,4R)-1-(methylsulfonyl)-4-((7-(3-morpholino-1H-pyrazol-4-yl)quinazolin-2-yl)amino)piperidin-3-ol;
(3R,4R)-4-((7-(3-((dimethylamino)methyl)-1H-pyrazol-4-yl)quinazolin-2-yl)amino)-1-(methylsulfonyl)piperidin-3-ol;
methyl (3R,4R)-4-((7-(3-(cyclopropylmethyl)-1H-pyrazol-4-yl)quinazolin-2-yl)amino)-3-hydroxypiperidine-1-carboxylate;
methyl (3R,4R)-4-((7-(1-cyclopropyl-1H-imidazol-5-yl)quinazolin-2-yl)amino)-3-hydroxypiperidine-1-carboxylate;
methyl (3R,4R)-4-((7-(1-ethyl-1H-imidazol-5-yl)quinazolin-2-yl)amino)-3-hydroxypiperidine-1-carboxylate;
methyl (3R,4R)-3-hydroxy-4-((7-(3-morpholino-1H-pyrazol-4-yl)quinazolin-2-yl)amino)piperidine-1-carboxylate;
methyl (3R,4R)-3-hydroxy-4-((7-(3-(tetrahydro-2H-pyran-4-yl)-1H-pyrazol-4-yl)quinazolin-2-yl)amino)piperidine-1-carboxylate;
methyl (3R,4R)-3-hydroxy-4-((7-(3-(2-hydroxyethyl)-1H-pyrazol-4-yl)quinazolin-2-yl)amino)piperidine-1-carboxylate;
methyl (3R,4R)-4-((7-(3-(4-(azetidin-1-ylmethyl)-3-fluorophenyl)-1H-pyrazol-4-yl)quinazolin-2-yl)amino)-3-hydroxypiperidine-1-carboxylate;
methyl (3R,4R)-4-((7-(3-(4-((dimethylamino)methyl)-3-fluorophenyl)-1H-pyrazol-4-yl)quinazolin-2-yl)amino)-3-hydroxypiperidine-1-carboxylate;
(3R,4R)-3-hydroxy-N,N-dimethyl-4-((7-(3-methyl-1H-pyrazol-4-yl)quinazolin-2-yl)amino)piperidine-1-sulfonamide;
(3R,4R)-4-((8-fluoro-7-(3-methyl-1H-pyrazol-4-yl)quinazolin-2-yl)amino)-1-(methylsulfonyl)piperidin-3-ol;
N-((3R,4R)-3-methoxy-1-(methylsulfonyl)piperidin-4-yl)-7-(3-methyl-1H-pyrazol-4-yl)quinazolin-2-amine;
(3R,4R)-1-(cyclopropylsulfonyl)-4-((7-(3-ethyl-1H-pyrazol-4-yl)-6,8-difluoroquinazolin-2-yl)amino)piperidin-3-ol;
(3R,4R)-4-((7-(3-methyl-1H-pyrazol-4-yl)pyrido[2,3-d]pyrimidin-2-yl)amino)-1-(methylsulfonyl)piperidin-3-ol;
(3R,4R)-4-((7-(1H-pyrazol-4-yl)pyrido[4,3-d]pyrimidin-2-yl)amino)-1-(methylsulfonyl)piperidin-3-ol;
(3R,4R)-4-((7-(3-methyl-1H-pyrazol-4-yl)pyrido[4,3-d]pyrimidin-2-yl)amino)-1-(methylsulfonyl)piperidin-3-ol;
(3R,4R)-4-((7-(3-isopropyl-1H-pyrazol-4-yl)pyrido[4,3-d]pyrimidin-2-yl)amino)-1-(methylsulfonyl)piperidin-3-ol;
(3R,4R)-4-((7-(1-(2,2-difluoroethyl)-1H-imidazol-5-yl)quinazolin-2-yl)amino)-1-(methylsulfonyl)piperidin-3-ol;
(3R,4R)-4-((7-(3-(1-methyl-1,2,3,6-tetrahydropyridin-4-yl)-1H-pyrazol-4-yl)quinazolin-2-yl)amino)-1-(methylsulfonyl)piperidin-3-ol;
(3R,4R)-4-((7-(3-(1-methylpiperidin-4-yl)-1H-pyrazol-4-yl)quinazolin-2-yl)amino)-1-(methylsulfonyl)piperidin-3-ol;
(3R,4R)-4-((7-(3-(2-methoxypyridin-4-yl)-1H-pyrazol-4-yl)quinazolin-2-yl)amino)-1-(methylsulfonyl)piperidin-3-ol;
(3R,4R)-4-((7-(5-(3-methoxyprop-1-yn-1-yl)-1H-pyrazol-4-yl)quinazolin-2-yl)amino)-1-(methylsulfonyl)piperidin-3-ol;
(3R,4R)-1-(cyclopropylsulfonyl)-4-((6,8-difluoro-7-(3-methyl-1H-pyrazol-4-yl)quinazolin-2-yl)amino)piperidin-3-ol;
(3R,4R)-4-((7-(3-ethyl-1H-pyrazol-4-yl)-6,8-difluoroquinazolin-2-yl)amino)-1-(methylsulfonyl)piperidin-3-ol;
(3R,4R)-4-((6,8-difluoro-7-(3-methyl-1H-pyrazol-4-yl)quinazolin-2-yl)amino)-1-(methylsulfonyl)piperidin-3-ol;
(3R,4R)-4-((6,8-difluoro-7-(1-methyl-1H-imidazol-5-yl)quinazolin-2-yl)amino)-1-(methylsulfonyl)piperidin-3-ol;
(3R,4R)-4-((6-fluoro-7-(3-methyl-1H-pyrazol-4-yl)quinazolin-2-yl)amino)-1-(methylsulfonyl)piperidin-3-ol;
(3R,4R)-4-((6-fluoro-7-(1H-pyrazol-4-yl)quinazolin-2-yl)amino)-1-(methylsulfonyl)piperidin-3-ol;
(3R,4R)-4-((7-(3-cyclopropyl-1H-pyrazol-4-yl)-6-fluoroquinazolin-2-yl)amino)-1-(methylsulfonyl)piperidin-3-ol;
(3R,4R)-4-((7-(3-(difluoromethyl)-1H-pyrazol-4-yl)-6-fluoroquinazolin-2-yl)amino)-1-(methylsulfonyl)piperidin-3-ol (3R,4R)-4-((6-fluoro-7-(3-fluoro-1H-pyrazol-4-yl)quinazolin-2-yl)amino)-1-(methylsulfonyl)piperidin-3-ol;
(3R,4R)-4-((6-fluoro-7-(1-methyl-1H-imidazol-5-yl)quinazolin-2-yl)amino)-1-(methylsulfonyl)piperidin-3-ol;
(3R,4R)-1-(cyclopropylsulfonyl)-4-((6-fluoro-7-(1-methyl-1H-imidazol-5-yl)quinazolin-2-yl)amino)piperidin-3-ol;
(3R,4R)-4-((6-fluoro-7-(1-methyl-1H-imidazol-5-yl)quinazolin-2-yl)amino)-3-hydroxypiperidin-1-yl)(1-(trifluoromethyl)cyclopropyl)methanone;
(1-(difluoromethyl)cyclopropyl)((3R,4R)-4-((6-fluoro-7-(1-methyl-1H-imidazol-5-yl)quinazolin-2-yl)amino)-3-hydroxypiperidin-1-yl)methanone;
((3R,4R)-4-((6-fluoro-7-(1-methyl-1H-imidazol-5-yl)quinazolin-2-yl)amino)-3-hydroxypiperidin-1-yl)(1-methylcyclopropyl)methanone;
(3R,4R)-1-(cyclopropylsulfonyl)-4-((6-fluoro-7-(1-methyl-1H-pyrazol-5-yl)quinazolin-2-yl)amino)piperidin-3-ol;
(3R,4R)-4-((5-fluoro-7-(1-methyl-1H-imidazol-5-yl)quinazolin-2-yl)amino)-1-(methylsulfonyl)piperidin-3-ol;
(3R,4R)-4-((5-fluoro-7-(3-methyl-1H-pyrazol-4-yl)quinazolin-2-yl)amino)-1-(methylsulfonyl)piperidin-3-ol;
(3R,4R)-4-((5-fluoro-7-(1H-pyrazol-4-yl)quinazolin-2-yl)amino)-1-(methylsulfonyl)piperidin-3-ol;
(3R,4R)-4-((6-fluoro-7-(1-(2-(methylamino)ethyl)-1H-pyrazol-4-yl)quinazolin-2-yl)amino)-1-(methylsulfonyl)piperidin-3-ol;
(3R,4R)-4-((6,8-difluoro-7-(1-(2-(methylamino)ethyl)-1H-pyrazol-4-yl)quinazolin-2-yl)amino)-1-(methylsulfonyl)piperidin-3-ol;
(3R,4R)-4-((7-(1-(2-(methylamino)ethyl)-1H-pyrazol-4-yl)quinazolin-2-yl)amino)-1-(methylsulfonyl)piperidin-3-ol;
((3R,4R)-4-((6-fluoro-7-(1-(2-(methylamino)ethyl)-1H-pyrazol-4-yl)quinazolin-2-yl)amino)-3-hydroxypiperidin-1-yl)(1-methylcyclopropyl)methanone;
(3,3-difluorocyclobutyl)((3R,4R)-4-((6-fluoro-7-(1-(2-(methylamino)ethyl)-1H-pyrazol-4-yl)quinazolin-2-yl)amino)-3-hydroxypiperidin-1-yl)methanone;
2,2-difluoroethyl (3R,4R)-4-((6-fluoro-7-(1-(2-(methylamino)ethyl)-1H-pyrazol-4-yl)quinazolin-2-yl)amino)-3-hydroxypiperidine-1-carboxylate;
((3R,4R)-4-((6-fluoro-7-(1-(2-(methylamino)ethyl)-1H-pyrazol-4-yl)quinazolin-2-yl)amino)-3-hydroxypiperidin-1-yl)(1-(trifluoromethyl)cyclobutyl)methanone;
(1-(difluoromethyl)cyclopropyl)((3R,4R)-4-((6-fluoro-7-(1-(2-(methylamino)ethyl)-1H-pyrazol-4-yl)quinazolin-2-yl)amino)-3-hydroxypiperidin-1-yl)methanone;
methyl (3R,4R)-4-((6-fluoro-7-(1H-pyrazol-4-yl)quinazolin-2-yl)amino)-3-hydroxypiperidine-1-carboxylate;
methyl (3R,4R)-4-((7-(3-ethyl-1H-pyrazol-4-yl)-6-fluoroquinazolin-2-yl)amino)-3-hydroxypiperidine-1-carboxylate; and
methyl (3R,4R)-4-((6-fluoro-7-(3-methyl-1H-pyrazol-4-yl)quinazolin-2-yl)amino)-3-hydroxypiperidine-1-carboxylate;
or a pharmaceutically acceptable salt thereof.

47. A pharmaceutical composition comprising the compound of claim 1, or a pharmaceutically acceptable salt thereof, and a pharmaceutically acceptable carrier.

48. A method of inhibiting CDK4, comprising contacting the CDK4 with the compound of claim 1, or a pharmaceutically acceptable salt thereof.

49. A method of inhibiting CDK4 in a patient, comprising administering to the patient a compound of claim 1, or a pharmaceutically acceptable salt thereof.

50. A method of treating a disease or disorder associated with CDK4 in a patient, comprising administering to the patient a therapeutically effective amount of the compound of claim 1, or pharmaceutically acceptable salt thereof.

51. The method of claim 51, wherein the disease or disorder is cancer.

52. The method of claim 52, wherein the cancer is selected from breast cancer, ovarian cancer, a gynecological cancer, a gastrointestinal cancer, prostate cancer, melanoma, colorectal cancer, endometrial cancer, esophageal cancer, gastric cancer, liposarcoma, Ewing sarcoma, or melanoma.

53. The method of claim 50, wherein the administering further comprises administering one or more additional pharmaceutical agents.
